(19) United States
(12) Patent Application Publication
(10) Pub. No.: US 2025/0101120 A1
(43) Pub. Date: Mar. 27, 2025

(54) ANTIBODIES SPECIFICALLY RECOGNIZING FASL AND USES THEREOF

(71) Applicant: BEIJING SOLOBIO GENETECHNOLOGY CO., LTD., Beijing (CN)

(72) Inventors: Guangfei WANG, Beijing (CN); Xiaoye REN, Beijing (CN); Zhong LI, Beijing (CN)

(21) Appl. No.: 18/730,785

(22) PCT Filed: Jan. 16, 2023
(86) PCT No.: PCT/CN2023/072293

§ 371 Date: Jul. 22, 2024

(30) Foreign Application Priority Data
Jan. 21, 2022 (CN) 202210069818.3

(51) Int. Cl. C07K 16/28; A61K 39/00; A61K 45/06; A61P 1/16

(52) U.S. Cl. CPC C07K 16/2875; A61K 45/06; A61P 1/16; A61K 2039/505

(57) ABSTRACT

This application pertains to antibody or antigen binding fragment that specifically recognizing FasL, and methods of manufacture and uses thereof.

### CROSS REFERENCE TO RELATED APPLICATIONS

This application claims priority to Chinese Patent Application No. 202210069818.3, filed on Jan. 21, 2022 and entitled “ANTIBODIES SPECIFICALLY RECOGNIZING FASL AND USES THEREOF”, the contents of which are incorporated herein by reference in their entirety.

### REFERENCE TO AN ELECTRONIC SEQUENCE LISTING

The contents of the electronic sequence listing (file name: CN_202201057572_SEQLIST.xml, date recorded: 2022.08.03, size: 53 KB) is herein incorporated by reference in its entirety.

### FIELD OF THE APPLICATION

This application pertains to antibody or antigen binding fragment that specifically recognizes FasL, and methods of manufacture and uses thereof.

### BACKGROUND OF THE APPLICATION

FasL (CD95L) is a transmembrane protein and proapoptotic member of the tumor necrosis factor (TNF) superfamily. The FasL extracellular region contains a ligand dimer and receptor binding region (TNF homology domain, THD), while the FasL intracellular region is involved in multiple signaling pathways, especially as a T cell receptor co-stimulatory molecule during T-cell activation (Calmon-Hamaty, Flavia et al. Cytokine vol. 75, 2 (2015): 228-33.). The intracellular portion of FasL contains an extended polyproline region enabling interaction with proteins having proline-binding motifs, such as Src homology 3 (SH3) and WW domains (Wenzel, J et al. FEBS letters vol. 509, 2 (2001): 255-62.; Blott, E J et al. Journal of cell science vol. 114,Pt 13 (2001): 2405-16.). Furthermore, several tyrosine phosphorylation sites and a “double” casein kinase phosphorylation motif are present. FasL occurs in two forms, membrane-bound and soluble. The soluble form is generated by alternative splicing or proteolytic processing of the membrane-bound form. The expression of FasL is tightly controlled and restricted in physiological conditions to innate and adaptive immune system cells as well as to immune privilege sites, such as the eyes, the placenta, or the testis (Stenqvist, Ann-Christin et al. Journal of immunology (Baltimore, Md.: 1950) vol. 191, 11 (2013): 5515-23.). FasL is expressed at the surface of the two main immune effector cells, i.e., activated T cells and natural killer (NK) cells, but also on macrophages, neutrophils, and dendritic cells (Kiener, P A et al. The Journal of experimental medicine vol. 185, 8 (1997): 1511-6.; Liles, W C et al. The Journal of experimental medicine vol. 184, 2 (1996): 429-40. doi:10.1084/jem.184.2.429.). The expression of FasL can be induced in response to TCR (T cell receptor) activation, but also upon cytokine stimulation particularly interferons (INF) through transcriptional regulation (Tsutsui, H et al. Journal of immunology (Baltimore, Md.: 1950) vol. 157, 9 (1996): 3967-73.) The Fas receptor (CD95/APO-1) is a member of the TNF receptor superfamily. There are two forms of Fas protein, membrane-bound and soluble, and Fas primarily exists in the membrane-bound form. However, due to the lack of a transmembrane domain, soluble Fas cannot interact with FasL to induce apoptosis, and it thus plays a regulatory role in apoptosis, such as inhibiting membrane-bound Fas (mFas) induced apoptosis (Jee, Youngheun et al. Journal of veterinary science vol. 11, 2 (2010): 115-9.). The extracellular domain of mFas contains three cysteine rich domains (CRDs), the structural hallmark of the TNF receptor family (Zhang, Gongyi. Current opinion in structural biology vol. 14, 2 (2004): 154-60.). The C-terminal of the intracellular domain of Fas comprises the death domain (DD), which is essential for apoptosis induction and characteristic for the subgroup of death receptors (Chan, F K et al. Science (New York, N.Y.) vol. 288, 5475 (2000): 2351-4.). Fas is expressed in multiple organ tissues, especially peripheral T and B lymphocytes, NK cells, mononuclear cells, fibroblasts, endothelial cells, epithelial cells, etc (Wang, Mei, and Ping Su. Systems biology in reproductive medicine vol. 64, 2 (2018): 93-102.).

In the Fas-mediated apoptotic pathway, binding of FasL drives Fas clustering and binding of Fas to FADD. FADD recruits caspase-8 and caspase-10 to form the death-inducing signaling complex (DISC) (Wilson, Nicholas S et al. Nature immunology vol. 10, 4 (2009): 348-55.). DISC is activated by specific post-translational modifications of the death receptor (DR), such as palmitoylation and O-linked glycosylation (Muppidi, Jagan R, and Richard M Siegel. Nature immunology vol. 5, 2 (2004): 182-9.; Wagner, Klaus W et al. Nature medicine vol. 13, 9 (2007): 1070-7.). The DISC mediates autocatalytic processing and activation of caspase-8 and caspase-10, which propagate the death signal either through proteolysis of e□ ector caspases such as caspases-3, caspase-6, and caspase-7. In type I cells such as thymocytes, the effect of caspases is sufficient to induce apoptosis. By contrast, apoptosis requires caspase-8-mediated cleavage of BH3-interacting domain death agonist (Bid), which is a BH3-only protein that can promote the permeabilization of mitochondrial outer membranes and release of cytochrome c in type II cells such as B cells. Upon release from mitochondria, cytochrome c acts as a cofactor for the assembly of a cytosolic caspase-activating complex called the apoptosome, which propagates the caspase activation cascade (Wagner, Klaus W et al. Nature medicine vol. 13, 9 (2007): 1070-7.).

Cell death is not the only cellular response emanating from Fas activation. Fas is also capable to induce NF-κB signaling. Acting downstream of cellular caspase-8-like inhibitory protein (cFLIP) and TRAF2 in the Fas signaling pathway, T-cell proliferation is induced through NF-κB activation (Kataoka, Takao, and Jürg Tschopp. Molecular and cellular biology vol. 24, 7 (2004): 2627-36.). The cFLIP N-terminal cleavage products p43-FLIP and p22-FLIP induce NF-κB activation by binding to the IKK complex (Golks, Alexander et al. The Journal of experimental medicine vol. 203, 5 (2006): 1295-305.; Krammer, Peter H et al. Nature reviews. Immunology vol. 7, 7 (2007): 532-42.). Further, overexpression of cFLIP inhibits Fas-induced apoptosis of activated T cells (Van Parijs, L et al. “Autoimmunity as a consequence of retrovirus-mediated expression of C-FLIP in lymphocytes.” Immunity vol. 11, 6 (1999): 763-70; Kirchhoff, S et al. Journal of immunology (Baltimore, Md.: 1950) vol. 165, 11 (2000): 6293-300.). Moreover, Fas signaling regulates peripheral T cell homeostasis by modulating the equilibrium between proliferation and cell death, for example, in naive and memory T cell subsets (Jaleco, Sara et al. Journal of immunology (Baltimore, Md.: 1950) vol. 171, 1 (2003): 61-8.). Therefore, homeostasis of peripheral T cells may be maintained by the dual outcomes of FasL/Fas signaling.

Fas-FasL signaling pathway is associated with the occurrence and development of many diseases, including autoimmune diseases, transplant rejection, spinal cord injury, spesis and so on. Therefore, it is critical to develop inhibitors that target FasL.

At present, there have been reports the inhibitors targeting FasL, such as antagonistic antibodies against hFAS ligand and its use is disclosed in Chinese patent CN1283662C; anti-FasL antibody 119-4A (used as a control antibody in the embodiments of this application) and its use is disclosed in Chinese patent CN108290950B; the Fas-Fc fusion protein APG101 (as a contrast in the embodiments of this application) and its use is disclosed in the Chinese patent CN104662039B. High affinity, high bioactivity anti-FasL antibodies are still needed in the current treatment field.

The disclosures of all publications, patents, patent applications and published patent applications referred to herein are hereby incorporated herein by reference in their entirety.

### BRIEF SUMMARY OF THE APPLICATION

In some embodiments, there is provided an isolated anti-FasL antibody comprising: a V_{H} comprising an HC-CDR1, an HC-CDR2, and an HC-CDR3 of a V_{H} comprising the amino acid sequence of SEQ ID NO: 19; and a V_{L} comprising an LC-CDR1, an LC-CDR2, and an LC-CDR3 of a V_{L} comprising the amino acid sequence of SEQ ID NO: 38.

In some embodiments, there is provided an isolated anti-FasL antibody comprising: a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7, or a variant thereof comprising up to about 5 amino acid substitutions in the HC-CDRs; and a V_{L} comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16, or a variant thereof comprising up to about 5 amino acid substitutions in the LC-CDRs.

In some embodiments, according to any one of the isolated anti-FasL antibodies described above, the isolated anti-FasL antibody comprises: a V_{H} comprising the amino acid sequence of any one of SEQ ID NOs: 19-25, or a variant thereof having at least about 80% sequence identity to the amino acid sequence of any one of SEQ ID NOs: 19-25; and a V_{L} comprising the amino acid sequence of any one of SEQ ID NOs: 38-42, or a variant thereof having at least about 80% sequence identity to the amino acid sequence of any one of SEQ ID NOs: 38-42.

In some embodiments, there is provided an isolated anti-FasL antibody comprising: (i) a V_{H} comprising the amino acid sequence of SEQ ID NO: 19, or a variant thereof having at least about 80% sequence identity to the amino acid sequence of SEQ ID NO: 19; and a V_{L} comprising the amino acid sequence of SEQ ID NO: 38, or a variant thereof having at least about 80% sequence identity to the amino acid sequence of SEQ ID NO: 38; (ii) a V_{H} comprising the amino acid sequence of SEQ ID NO: 20, or a variant thereof having at least about 80% sequence identity to the amino acid sequence of SEQ ID NO: 20; and a V_{L} comprising the amino acid sequence of SEQ ID NO: 39, or a variant thereof having at least about 80% sequence identity to the amino acid sequence of SEQ ID NO: 39; (iii) a V_{H} comprising the amino acid sequence of SEQ ID NO: 21, or a variant thereof having at least about 80% sequence identity to the amino acid sequence of SEQ ID NO: 21; and a V_{L} comprising the amino acid sequence of SEQ ID NO: 40, or a variant thereof having at least about 80% sequence identity to the amino acid sequence of SEQ ID NO: 40; (iv) a V_{H} comprising the amino acid sequence of SEQ ID NO: 22, or a variant thereof having at least about 80% sequence identity to the amino acid sequence of SEQ ID NO: 22; and a V_{L} comprising the amino acid sequence of SEQ ID NO: 40, or a variant thereof having at least about 80% sequence identity to the amino acid sequence of SEQ ID NO: 40; (v) a V_{H} comprising the amino acid sequence of SEQ ID NO: 23, or a variant thereof having at least about 80% sequence identity to the amino acid sequence of SEQ ID NO: 23; and a V_{L} comprising the amino acid sequence of SEQ ID NO: 40, or a variant thereof having at least about 80% sequence identity to the amino acid sequence of SEQ ID NO: 40; (vi) a V_{H} comprising the amino acid sequence of SEQ ID NO: 24, or a variant thereof having at least about 80% sequence identity to the amino acid sequence of SEQ ID NO: 24; and a V_{L} comprising the amino acid sequence of SEQ ID NO: 40, or a variant thereof having at least about 80% sequence identity to the amino acid sequence of SEQ ID NO: 40; (vii) a V_{H} comprising the amino acid sequence of SEQ ID NO: 25, or a variant thereof having at least about 80% sequence identity to the amino acid sequence of SEQ ID NO: 25; and a V_{L} comprising the amino acid sequence of SEQ ID NO: 40, or a variant thereof having at least about 80% sequence identity to the amino acid sequence of SEQ ID NO: 40; (viii) a V_{H} comprising the amino acid sequence of SEQ ID NO: 21, or a variant thereof having at least about 80% sequence identity to the amino acid sequence of SEQ ID NO: 21; and a V_{L} comprising the amino acid sequence of SEQ ID NO: 41, or a variant thereof having at least about 80% sequence identity to the amino acid sequence of SEQ ID NO: 41; (ix) a V_{H} comprising the amino acid sequence of SEQ ID NO: 22, or a variant thereof having at least about 80% sequence identity to the amino acid sequence of SEQ ID NO: 22; and a V_{L} comprising the amino acid sequence of SEQ ID NO: 41, or a variant thereof having at least about 80% sequence identity to the amino acid sequence of SEQ ID NO: 41; (x) a V_{H} comprising the amino acid sequence of SEQ ID NO: 23, or a variant thereof having at least about 80% sequence identity to the amino acid sequence of SEQ ID NO: 23; and a V_{L} comprising the amino acid sequence of SEQ ID NO: 41, or a variant thereof having at least about 80% sequence identity to the amino acid sequence of SEQ ID NO: 41; (xi) a V_{H} comprising the amino acid sequence of SEQ ID NO: 24, or a variant thereof having at least about 80% sequence identity to the amino acid sequence of SEQ ID NO: 24; and a V_{L} comprising the amino acid sequence of SEQ ID NO: 41, or a variant thereof having at least about 80% sequence identity to the amino acid sequence of SEQ ID NO: 41; (xii) a V_{H} comprising the amino acid sequence of SEQ ID NO: 25, or a variant thereof having at least about 80% sequence identity to the amino acid sequence of SEQ ID NO: 25; and a V_{L} comprising the amino acid sequence of SEQ ID NO: 41, or a variant thereof having at least about 80% sequence identity to the amino acid sequence of SEQ ID NO: 41; (xiii) a V_{H} comprising the amino acid sequence of SEQ ID NO: 21, or a variant thereof having at least about 80% sequence identity to the amino acid sequence of SEQ ID NO: 21; and a V_{L} comprising the amino acid sequence of SEQ ID NO: 42, or a variant thereof having at least about 80% sequence identity to the amino acid sequence of SEQ ID NO: 42; (xiv) a V_{H} comprising the amino acid sequence of SEQ ID NO: 22, or a variant thereof having at least about 80% sequence identity to the amino acid sequence of SEQ ID NO: 22; and a V_{L} comprising the amino acid sequence of SEQ ID NO: 42, or a variant thereof having at least about 80% sequence identity to the amino acid sequence of SEQ ID NO: 42; (xv) a V_{H} comprising the amino acid sequence of SEQ ID NO: 23, or a variant thereof having at least about 80% sequence identity to the amino acid sequence of SEQ ID NO: 23; and a V_{L} comprising the amino acid sequence of SEQ ID NO: 42, or a variant thereof having at least about 80% sequence identity to the amino acid sequence of SEQ ID NO: 42; (xvi) a V_{H} comprising the amino acid sequence of SEQ ID NO: 24, or a variant thereof having at least about 80% sequence identity to the amino acid sequence of SEQ ID NO: 24; and a V_{L} comprising the amino acid sequence of SEQ ID NO: 42, or a variant thereof having at least about 80% sequence identity to the amino acid sequence of SEQ ID NO: 42; (xvii) a V_{H} comprising the amino acid sequence of SEQ ID NO: 25, or a variant thereof having at least about 80% sequence identity to the amino acid sequence of SEQ ID NO: 25; and a V_{L} comprising the amino acid sequence of SEQ ID NO: 42, or a variant thereof having at least about 80% sequence identity to the amino acid sequence of SEQ ID NO: 42.

In some embodiments, there is provided an isolated anti-FasL antibody comprising: a V_{H} comprising an HC-CDR1, an HC-CDR2, and an HC-CDR3 of a V_{H} comprising the amino acid sequence of SEQ ID NO: 26; and a V_{L} comprising an LC-CDR1, an LC-CDR2, and an LC-CDR3 of a V_{L} comprising the amino acid sequence of SEQ ID NO: 43.

In some embodiments, there is provided an isolated anti-FasL antibody comprising: a V_{H} comprising an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8, or a variant thereof comprising up to about 5 amino acid substitutions in the HC-CDRs; and a V_{L} comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17, or a variant thereof comprising up to about 5 amino acid substitutions in the LC-CDRs.

In some embodiments, according to any one of the isolated anti-FasL antibodies described above, the isolated anti-FasL antibody comprises: a V_{H} comprising the amino acid sequence of any one of SEQ ID NOs: 26-28, or a variant thereof having at least about 80% sequence identity to the amino acid sequence of any one of SEQ ID NOs: 26-28; and a V_{L} comprising the amino acid sequence of any one of SEQ ID NOs: 43-45, or a variant thereof having at least about 80% sequence identity to the amino acid sequence of any one of SEQ ID NOs: 43-45.

In some embodiments, there is provided an isolated anti-FasL antibody comprising: (i) a V_{H} comprising the amino acid sequence of SEQ ID NO: 26, or a variant thereof having at least about 80% sequence identity to the amino acid sequence of SEQ ID NO: 26; and a V_{L} comprising the amino acid sequence of SEQ ID NO: 43, or a variant thereof having at least about 80% sequence identity to the amino acid sequence of SEQ ID NO: 43; (ii) a V_{H} comprising the amino acid sequence of SEQ ID NO: 27, or a variant thereof having at least about 80% sequence identity to the amino acid sequence of SEQ ID NO: 27; and a V_{L} comprising the amino acid sequence of SEQ ID NO: 44, or a variant thereof having at least about 80% sequence identity to the amino acid sequence of SEQ ID NO: 44; (iii) a V_{H} comprising the amino acid sequence of SEQ ID NO: 28, or a variant thereof having at least about 80% sequence identity to the amino acid sequence of SEQ ID NO: 28; and a V_{L} comprising the amino acid sequence of SEQ ID NO: 44, or a variant thereof having at least about 80% sequence identity to the amino acid sequence of SEQ ID NO: 44; (iv) a V_{H} comprising the amino acid sequence of SEQ ID NO: 27, or a variant thereof having at least about 80% sequence identity to the amino acid sequence of SEQ ID NO: 27; and a V_{L} comprising the amino acid sequence of SEQ ID NO: 45, or a variant thereof having at least about 80% sequence identity to the amino acid sequence of SEQ ID NO: 45; (v) a V_{H} comprising the amino acid sequence of SEQ ID NO: 28, or a variant thereof having at least about 80% sequence identity to the amino acid sequence of SEQ ID NO: 28; and a V_{L} comprising the amino acid sequence of SEQ ID NO: 45, or a variant thereof having at least about 80% sequence identity to the amino acid sequence of SEQ ID NO: 45.

In some embodiments, there is provided an isolated anti-FasL antibody comprising: a V_{H} comprising an HC-CDR1, an HC-CDR2, and an HC-CDR3 of a V_{H} comprising the amino acid sequence of SEQ ID NO: 31; and a V_{L} comprising an LC-CDR1, an LC-CDR2, and an LC-CDR3 of a V_{L} comprising the amino acid sequence of SEQ ID NO: 48.

In some embodiments, there is provided an isolated anti-FasL antibody comprising: a V_{H} comprising an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9, or a variant thereof comprising up to about 5 amino acid substitutions in the HC-CDRs; and a V_{L} comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18, or a variant thereof comprising up to about 5 amino acid substitutions in the LC-CDRs.

In some embodiments, according to any one of the isolated anti-FasL antibodies described above, the isolated anti-FasL antibody comprises: a V_{H} comprising the amino acid sequence of any one of SEQ ID NOs: 29-37, or a variant thereof having at least about 80% sequence identity to the amino acid sequence of any one of SEQ ID NOs: 29-37; and a V_{L} comprising the amino acid sequence of any one of SEQ ID NOs: 46-52, or a variant thereof having at least about 80% sequence identity to the amino acid sequence of any one of SEQ ID NOs: 46-52.

In some embodiments, there is provided an isolated anti-FasL antibody comprising: (i) a V_{H} comprising the amino acid sequence of SEQ ID NO: 29, or a variant thereof having at least about 80% sequence identity to the amino acid sequence of SEQ ID NO: 29; and a V_{L} comprising the amino acid sequence of SEQ ID NO: 46, or a variant thereof having at least about 80% sequence identity to the amino acid sequence of SEQ ID NO: 46; (ii) a V_{H} comprising the amino acid sequence of SEQ ID NO: 30, or a variant thereof having at least about 80% sequence identity to the amino acid sequence of SEQ ID NO: 30; and a V_{L} comprising the amino acid sequence of SEQ ID NO: 47, or a variant thereof having at least about 80% sequence identity to the amino acid sequence of SEQ ID NO: 47; (iii) a V_{H} comprising the amino acid sequence of SEQ ID NO: 31, or a variant thereof having at least about 80% sequence identity to the amino acid sequence of SEQ ID NO: 31; and a V_{L} comprising the amino acid sequence of SEQ ID NO: 48, or a variant thereof having at least about 80% sequence identity to the amino acid sequence of SEQ ID NO: 48; (iv) a V_{H} comprising the amino acid sequence of SEQ ID NO: 31, or a variant thereof having at least about 80% sequence identity to the amino acid sequence of SEQ ID NO: 31; and a V_{L} comprising the amino acid sequence of SEQ ID NO: 49, or a variant thereof having at least about 80% sequence identity to the amino acid sequence of SEQ ID NO: 49; (v) a V_{H} comprising the amino acid sequence of SEQ ID NO: 32, or a variant thereof having at least about 80% sequence identity to the amino acid sequence of SEQ ID NO: 32; and a V_{L} comprising the amino acid sequence of SEQ ID NO: 50, or a variant thereof having at least about 80% sequence identity to the amino acid sequence of SEQ ID NO: 50; (vi) a V_{H} comprising the amino acid sequence of SEQ ID NO: 34, or a variant thereof having at least about 80% sequence identity to the amino acid sequence of SEQ ID NO: 34; and a V_{L} comprising the amino acid sequence of SEQ ID NO: 50, or a variant thereof having at least about 80% sequence identity to the amino acid sequence of SEQ ID NO: 50; (vii) a V_{H} comprising the amino acid sequence of SEQ ID NO: 35, or a variant thereof having at least about 80% sequence identity to the amino acid sequence of SEQ ID NO: 35; and a V_{L} comprising the amino acid sequence of SEQ ID NO: 50, or a variant thereof having at least about 80% sequence identity to the amino acid sequence of SEQ ID NO: 50; (viii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 50, or a variant thereof having at least about 80% sequence identity to the amino acid sequence of SEQ ID NO: 50; (ix) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 50, or a variant thereof having at least about 80% sequence identity to the amino acid sequence of SEQ ID NO: 50; (x) a V_{H} comprising the amino acid sequence of SEQ ID NO: 32, or a variant thereof having at least about 80% sequence identity to the amino acid sequence of SEQ ID NO: 32; and a V_{L} comprising the amino acid sequence of SEQ ID NO: 51, or a variant thereof having at least about 80% sequence identity to the amino acid sequence of SEQ ID NO: 51; (xi) a V_{H} comprising the amino acid sequence of SEQ ID NO: 34, or a variant thereof having at least about 80% sequence identity to the amino acid sequence of SEQ ID NO: 34; and a V_{L} comprising the amino acid sequence of SEQ ID NO: 51, or a variant thereof having at least about 80% sequence identity to the amino acid sequence of SEQ ID NO: 51; (xii) a V_{H} comprising the amino acid sequence of SEQ ID NO: 35, or a variant thereof having at least about 80% sequence identity to the amino acid sequence of SEQ ID NO: 35; and a V_{L} comprising the amino acid sequence of SEQ ID NO: 51, or a variant thereof having at least about 80% sequence identity to the amino acid sequence of SEQ ID NO: 51; (xiii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 51, or a variant thereof having at least about 80% sequence identity to the amino acid sequence of SEQ ID NO: 51; (xiv) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 51, or a variant thereof having at least about 80% sequence identity to the amino acid sequence of SEQ ID NO: 51; (xv) a V_{H} comprising the amino acid sequence of SEQ ID NO: 32, or a variant thereof having at least about 80% sequence identity to the amino acid sequence of SEQ ID NO: 32; and a V_{L} comprising the amino acid sequence of SEQ ID NO: 52, or a variant thereof having at least about 80% sequence identity to the amino acid sequence of SEQ ID NO: 52; (xvi) a V_{H} comprising the amino acid sequence of SEQ ID NO: 34, or a variant thereof having at least about 80% sequence identity to the amino acid sequence of SEQ ID NO: 34; and a V_{L} comprising the amino acid sequence of SEQ ID NO: 52, or a variant thereof having at least about 80% sequence identity to the amino acid sequence of SEQ ID NO: 52; (xvii) a V_{H} comprising the amino acid sequence of SEQ ID NO: 35, or a variant thereof having at least about 80% sequence identity to the amino acid sequence of SEQ ID NO: 35; and a V_{L} comprising the amino acid sequence of SEQ ID NO: 52, or a variant thereof having at least about 80% sequence identity to the amino acid sequence of SEQ ID NO: 52; (xviii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 52, or a variant thereof having at least about 80% sequence identity to the amino acid sequence of SEQ ID NO: 52; or (xix) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 52, or a variant thereof having at least about 80% sequence identity to the amino acid sequence of SEQ ID NO: 52.

In some embodiments, there is provided an isolated anti-FasL antibody that competes with any one of the isolated anti-FasL antibodies described above for specifically binding to FasL. In some embodiments, there is provided an isolated anti-FasL antibody that specifically binds to the same epitope as any one of isolated anti-FasL antibodies described above.

In some embodiments according to any of the isolated anti-FasL antibodies described above, the isolated anti-FasL antibody comprises an Fc fragment. In some embodiments, the isolated anti-FasL antibody is a full-length IgG antibody. In some embodiments, the isolated anti-FasL antibody is a full-length IgG1 or IgG4 antibody. In some embodiments, the anti-FasL antibody is a chimeric, human, or humanized antibody. In some embodiments, the anti-FasL antibody is an antigen binding fragment selected from the group consisting of a Fab, a Fab′, a F(ab₂, a Fab′-SH, a single-chain Fv (scFv), an Fv fragment, a dAb, a Fd, a nanobody, a diabody, and a linear antibody.

In some embodiments, there is provided isolated nucleic acid molecule(s) that encodes any one of the anti-FasL antibodies described above. In some embodiments, there is provided a vector comprising any one of the nucleic acid molecules described above. In some embodiments, there is provided a host cell comprising any one of the anti-FasL antibodies described above, any one of the nucleic acid molecules described above, or any one of the vectors described above. In some embodiments, there is provided a method of producing an anti-FasL antibody, comprising: a) culturing any one of the host cells described above under conditions effective to express the anti-FasL antibody; and b) obtaining the expressed anti-FasL antibody from the host cell.

In some embodiments, there is provided a method of treating a disease or condition in an individual in need thereof, comprising administering to the individual an effective amount of any one of the anti-FasL antibodies described above. In some embodiments, there is provided the use of any one of the anti-FasL antibodies described herein for the preparation of pharmaceutical compositions for treating a disease or condition in an individual in need. In some embodiments, there is provided the use of any one of the anti-FasL antibodies described above, or a pharmaceutical composition comprising any one of anti-FasL antibodies described above in the manufacture of a medicament for treating a disease or condition. In some embodiments, the disease or condition is associated with the FasL-Fas signaling pathway, comprising inflammatory diseases, cancer or autoimmune diseases or condition. In some embodiments, the disease or condition is selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer.

Also provided are pharmaceutical compositions, kits and articles of manufacture comprising any one of the anti-FasL antibodies described above.

### BRIEF DESCRIPTION OF THE DRAWINGS

FIGS. 1A-1B show the inhibition of anti-FasL antibodies in OKT (CD 3 antibody) induced Jurkat cell self-killing.

FIGS. 2A-2B show the binding curves of the humanized antibodies of FL-M54 or FL-M78 to the human FasL antigen.

FIG. 3A shows the inhibition of humanized antibodies of FL-M78 in FasL antigen induced Jurkat cell apoptosis. FIG. 3B shows the inhibition of humanized antibodies of FL-M78 in FasL antigen induced HepG2 cell apoptosis.

FIG. 4 shows the inhibition of FL-M78 and its humanized antibodies in FasL antigen-induced NF-κB signaling pathway activation.

FIGS. 5A-5C show the activity of humanized antibodies FL-M54, FL-M78, or FL-M88 to inhibit OKT-induced apoptosis of Jurkat cells, respectively.

FIGS. 6A-6C show the activity of humanized antibodies FL-M54, FL-M78, or FL-M88 to inhibit PBMC killing Jurkat cells, respectively.

FIG. 7 shows the anti-FASL antibodies humFL-M78-1 and humFL-M78-2 significantly inhibited the elevation of ALT induced by exogenous FasL in a dose-dependent manner.

FIG. 8 shows the anti-FASL FL-M88, humFL-M88-1 and humFL-M88-3 significantly inhibited the elevation of ALT induced by APAP.

### DETAILED DESCRIPTION OF THE APPLICATION

The present application in one aspect provides an isolated anti-FasL antibody. By using a combination of selections on scFv phage libraries, appropriately designed biochemical and biological assays and antibody humanization, highly potent antibody molecules that bind to human FasL and inhibit the action of human FasL to its receptor have been identified. The results presented herein indicate that compared with the known anti-FasL antibody, the present application antibodies exhibit better biological activity.

The anti-FasL antibodies provided by the present application include, for example, full-length anti-FasL antibodies, anti-FasL scFvs, anti-FasL Fc fusion proteins, multi-specific (such as bispecific) anti-FasL antibodies, anti-FasL immunoconjugates, and the like.

On the other hand, there is provided an isolated anti-FasL antibody comprising: a V_{H} comprising an HC-CDR1 comprising the amino acid sequence of SEQ ID NO: 1, an HC-CDR2 comprising the amino acid sequence of SEQ ID NO: 1, and an HC-CDR3 comprising the amino acid sequence of SEQ ID NO: 7 or a variant thereof comprising up to about 5 amino acid substitutions in the HC-CDRs; and a V_{L} comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16, or a variant thereof comprising up to about 5 amino acid substitutions in the LC-CDRs.

On the other hand, there is provided an isolated anti-FasL antibody comprising: a V_{H} comprising an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8, or a variant thereof comprising up to about 5 amino acid substitutions in the HC-CDRs; and a V_{L} comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17, or a variant thereof comprising up to about 5 amino acid substitutions in the LC-CDRs.

On the other hand, there is provided an isolated anti-FasL antibody comprising: a V_{H} comprising an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9, or a variant thereof comprising up to about 5 amino acid substitutions in the HC-CDRs; and a V_{L} comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18, or a variant thereof comprising up to about 5 amino acid substitutions in the LC-CDRs.

Also provided are nucleic acids encoding the anti-FasL antibodies, compositions comprising the anti-FasL antibodies, and methods of making and using the anti-FasL antibodies.

### Definitions

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease) preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more of other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume for cancer). The methods of the application contemplate any one or more of these aspects of treatment.

The term “antibody” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain) or IgA2 (α2 heavy chain).

The term “antigen-binding fragment” as used herein includes an antibody fragment including, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody “competes” for binding to a target FasL with a second antibody when the first antibody inhibits target FasL binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731.

As used herein, the term “specifically binds”, “specifically recognizing”, or “is specific for” refers to measurable and reproducible interactions, such as binding between a target and an antibody that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its binding to other targets. In some embodiments, an antibody that specifically recognizes an antigen reacts with one or more antigenic determinants of the antigen with a binding affinity that is at least about 10 times its binding affinity for other targets.

An “isolated” anti-FasL antibody as used herein refers to an anti-FasL antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al, U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol 262:732-745 (1996); Abhinandan and Martin, Mol. Immunol, 45: 3832-3839 (2008); Lefranc M. P. et al., Dev. Comp. Immunol, 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al, Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibody” refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad Sci. USA, 81:6851-6855 (1984)).

“Fv” is the minimum antibody fragment which contains a complete antigen-recognition and binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv”, also abbreviated as “sFv” or “scFv”, are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues.

Furthermore, humanized antibodies can comprise residues that are not found in the recipient antibody or in the donor antibody. These modifications are made to further refine antibody performance. In general, the humanized antibody will comprise substantially at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Stuct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this application is one that binds to an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al, Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al, J. Immunol. 24:249 (1994)).

The term “FcRn” refers to the neonatal Fc receptor (FcRn). FcRn is structurally similar to major histocompatibility complex (MHC) and consists of an α-chain noncovalently bound to P2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues of C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5-fold to about 100-fold, e.g. from about 25 to about 50-fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (Cl q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-FasL antibody or composition as disclosed herein, is an amount sufficient to carry out a specifically stated purpose. An “effective amount” can be determined empirically and by known methods relating to the stated purpose.

The term “therapeutically effective amount” refers to an amount of an anti-FasL antibody or composition as disclosed herein, effective to “treat” a disease or disorder in an individual. As used herein, the term “effective amount” refers to a sufficient degree of severity and/or duration of treatment to reduce or ameliorate a disorder or one or more symptoms thereof; preventing the progression of the disorder; causing regression of the condition; preventing the recurrence, development, onset, or progression of one or more symptoms associated with the disorder; detecting a condition; or an amount that enhances or improves the prophylactic or therapeutic effect of another therapy (e.g., prophylactic or therapeutic agent). In the case of cancer, the therapeutically effective amount of the anti-FasL antibody or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-FasL antibody or composition as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient.

The anti-FASL antibody or its composition disclosed here can prevent or inhibit the binding of FasL to its receptor Fas to some extent. In addition to the apoptotic signaling pathway, Fas mediated non apoptotic signals (such as NF kB, MAPK, or PI3K) promote inflammation, contributes to carcinogenesis, and regulates immunological parameters (e.g., tumor-infiltrating T-cell populations). All of these activities may be inhibited by the antibodies described here.

As used herein, by “pharmaceutically acceptable” or “pharmacologically compatible” is meant a material that is not biological or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S.

### Food and Drug Administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a”, “an”, and “the” include plural referents unless the context clearly dictates otherwise.

### Anti-FasL Antibodies

In one aspect, the present application provides anti-FasL antibodies that specifically bind to human FasL. Anti-FasL antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to FasL. Contemplated anti-FasL antibodies include, for example, full-length anti-FasL antibodies (e.g., full-length IgG1 or IgG4), anti-FasL scFvs, anti-FasL Fc fusion proteins, multi-specific (such as bispecific) anti-FasL antibodies, anti-FasL immunoconjugates, and the like. In some embodiments, the anti-FasL antibody is a full-length antibody (e.g., full-length IgG1 or IgG4) or antigen-binding fragment thereof, which specifically binds to FasL. In some embodiments, the anti-FasL antibody is a Fab, a Fab′, a F(ab)′₂, a Fab′-SH, a single-chain Fv (scFv), an Fv fragment, a dAb, a Fd, a nanobody, a diabody, or a linear antibody. In some embodiments, reference to an antibody that specifically binds to FasL means that the antibody binds to FasL with an affinity that is at least about 10 times (including for example at least about any one of 10, 10², 10³, 10⁴, 10⁵, 10⁶, or 10⁷ times) more tightly than its binding affinity for a non-target. In some embodiments, the non-target is an antigen that is not FasL. Binding affinity can be determined by methods known in the art, such as ELISA, fluorescence activated cell sorting (FACS) analysis, or radioimmunoprecipitation assay (RIA). Kd can be determined by methods known in the art, such as surface plasmon resonance (SPR) assay or biolayer interferometry (BLI).

Although anti-FasL antibodies containing human sequences (e.g., human heavy and light chain variable domain sequences comprising human CDR sequences) are extensively discussed herein, non-human anti-FasL antibodies are also contemplated. In some embodiments, non-human anti-FasL antibodies comprise human CDR sequences from an anti-FasL antibody as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-FasL antibody includes an anti-FasL antibody generated by grafting one or more human CDR sequences as described herein onto a non-human framework sequence (e.g., a mouse or chicken framework sequence).

The complete amino acid sequence of an exemplary native human FasL comprises or consists of the amino acid sequence of SEQ ID NO: 57. The amino acid sequence of human FasL extracellular region comprises or consists of the amino acid sequence of SEQ ID NO: 58.

In some embodiments, the anti-FasL antibody described herein specifically recognizes an epitope within human FasL. In some embodiments, the anti-FasL antibody cross-reacts with FasL from species other than human. In some embodiments, the anti-FasL antibody is completely specific for human FasL and does not exhibit cross-reactivity with FasL from other non-human species.

In some embodiments, the anti-FasL antibody cross-reacts with at least one allelic variant of the FasL protein (or fragments thereof). In some embodiments, the allelic variant has up to about 30 (such as about any of 1, 2, 3, 4, 5, 6, 7, 8, 9, 10, 15, 20, 25, or 30) amino acid substitutions (such as a conservative substitution) when compared to the naturally occurring FasL (or fragments thereof). In some embodiments, the anti-FasL antibody does not cross-react with any allelic variants of the FasL protein (or fragments thereof).

In some embodiments, the anti-FasL antibody cross-reacts with at least one interspecies variant of the FasL protein. In some embodiments, for example, the FasL protein (or fragments thereof) is human FasL and the interspecies variant of the FasL protein (or fragments thereof) is a cynomolgus monkey variant thereof. In some embodiments, the anti-FasL antibody does not cross-react with any interspecies variants of the FasL protein.

In some embodiments, according to any of the anti-FasL antibodies described herein, the anti-FasL antibody comprises an antibody heavy chain constant region and an antibody light chain constant region. In some embodiments, the anti-FasL antibody comprises an IgG1 heavy chain constant region. In some embodiments, the anti-FasL antibody comprises an IgG2 heavy chain constant region. In some embodiments, the anti-FasL antibody comprises an IgG3 heavy chain constant region. In some embodiments, the anti-FasL antibody comprises an IgG4 heavy chain constant region. In some embodiments, the heavy chain constant region comprises (including consisting of or consisting essentially of) the amino acid sequence of SEQ ID NO: 53. In some embodiments, the heavy chain constant region comprises (including consisting of or consisting essentially of) the amino acid sequence of SEQ ID NO: 54. In some embodiments, the anti-FasL antibody comprises a kappa light chain constant region. In some embodiments, the light chain constant region comprises (including consisting of or consisting essentially of) the amino acid sequence of SEQ ID NO: 55. In some embodiments, the anti-FasL antibody comprises a lambda light chain constant region. In some embodiments, the light chain constant region comprises (including consisting of or consisting essentially of) the amino acid sequence of SEQ ID NO: 56. In some embodiments, the anti-FasL antibody comprises an antibody heavy chain variable domain and an antibody light chain variable domain.

In some embodiments, the anti-FasL antibody comprises a V_{H} comprising: an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7, or a variant thereof comprising up to about 5 amino acid substitutions in the HC-CDRs; and a V_{L} comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16, or a variant thereof comprising up to about 5 amino acid substitutions in the LC-CDRs.

In some embodiments, the anti-FasL antibody comprises a V_{H} comprising: an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7; and a V_{L} comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16.

In some embodiments, the anti-FasL antibody comprises a V_{H} comprising an HC-CDR1, an HC-CDR2 and an HC-CDR3 of the V_{H} comprising the amino acid sequence of SEQ ID NO: 19, and a V_{L} comprising an LC-CDR1, an LC-CDR2 and an LC-CDR3 of the V_{L} comprising the amino acid sequence of SEQ ID NO: 38.

In some embodiments, the anti-FasL antibody comprises a V_{H} comprising the amino acid sequence of any one of SEQ ID NOs: 19-25, or a variant thereof having at least about 80% (such as at least about any of 80%, 85%, 90%, 95%, 96%, 97%, 98%, or 99%) sequence identity to the amino acid sequence of any one of SEQ ID NOs: 19-25, and a V_{L} comprising the amino acid sequence of any one of SEQ ID NOs: 38-42, or a variant thereof having at least about 80% (such as at least about any of 80%, 85%, 90%, 95%, 96%, 97%, 98%, or 99%) sequence identity to the amino acid sequence of any one of SEQ ID NOs: 38-42. In some embodiments, the anti-FasL antibody comprises a V_{H} comprising the amino acid sequence of any one of SEQ ID NOs: 19-25, and a V_{L} comprising the amino acid sequence of any one of SEQ ID NOs: 38-42.

In some embodiments, the anti-FasL antibody comprises a V_{H} comprising the amino acid sequence of SEQ ID NO: 19, or a variant thereof having at least about 80% (such as at least about any of 80%, 85%, 90%, 95%, 96%, 97%, 98%, or 99%) sequence identity to the amino acid sequence of SEQ ID NO: 19, and a V_{L} comprising the amino acid sequence of SEQ ID NO: 38, or a variant thereof having at least about 80% (such as at least about any of 80%, 85%, 90%, 95%, 96%, 97%, 98%, or 99%) sequence identity to the amino acid sequence of SEQ ID NO: 38. In some embodiments, the anti-FasL antibody comprises a V_{H} comprising the amino acid sequence of SEQ ID NO: 19 and a V_{L} comprising the amino acid sequence of SEQ ID NO: 38.

In some embodiments, the anti-FasL antibody comprises a V_{H} comprising the amino acid sequence of SEQ ID NO: 20, or a variant thereof having at least about 80% (such as at least about any of 80%, 85%, 90%, 95%, 96%, 97/, 98%, or 99%) sequence identity to the amino acid sequence of SEQ ID NO: 20, and a V_{L} comprising the amino acid sequence of SEQ ID NO: 39, or a variant thereof having at least about 80% (such as at least about any of 80%, 85%, 90%, 95%, 96%, 97%, 98%, or 99%) sequence identity to the amino acid sequence of SEQ ID NO: 39. In some embodiments, the anti-FasL antibody comprises a V_{H} comprising the amino acid sequence of SEQ ID NO: 20 and a V_{L} comprising the amino acid sequence of SEQ ID NO: 39.

In some embodiments, the anti-FasL antibody comprises a V_{H} comprising the amino acid sequence of SEQ ID NO: 21, or a variant thereof having at least about 80% (such as at least about any of 80%, 85%, 90%, 95%, 96%, 97/, 98%, or 99%) sequence identity to the amino acid sequence of SEQ ID NO: 21, and a V_{L} comprising the amino acid sequence of SEQ ID NO: 40, or a variant thereof having at least about 80% (such as at least about any of 80%, 85%, 90%, 95%, 96%, 97%, 98%, or 99%) sequence identity to the amino acid sequence of SEQ ID NO: 40. In some embodiments, the anti-FasL antibody comprises a V_{H} comprising the amino acid sequence of SEQ ID NO: 21 and a V_{L} comprising the amino acid sequence of SEQ ID NO: 40.

In some embodiments, the anti-FasL antibody comprises a V_{H} comprising the amino acid sequence of SEQ ID NO: 22, or a variant thereof having at least about 80% (such as at least about any of 80%, 85%, 90%, 95%, 96%, 97/, 98%, or 99%) sequence identity to the amino acid sequence of SEQ ID NO: 22, and a V_{L} comprising the amino acid sequence of SEQ ID NO: 40, or a variant thereof having at least about 80% (such as at least about any of 80%, 85%, 90%, 95%, 96%, 97%, 98%, or 99%) sequence identity to the amino acid sequence of SEQ ID NO: 40. In some embodiments, the anti-FasL antibody comprises a V_{H} comprising the amino acid sequence of SEQ ID NO: 22 and a V_{L} comprising the amino acid sequence of SEQ ID NO: 40.

In some embodiments, the anti-FasL antibody comprises a V_{H} comprising the amino acid sequence of SEQ ID NO: 23, or a variant thereof having at least about 80% (such as at least about any of 80%, 85%, 90%, 95%, 96%, 97/, 98%, or 99%) sequence identity to the amino acid sequence of SEQ ID NO: 23, and a V_{L} comprising the amino acid sequence of SEQ ID NO: 40, or a variant thereof having at least about 80% (such as at least about any of 80%, 85%, 90%, 95%, 96%, 97%, 98%, or 99%) sequence identity to the amino acid sequence of SEQ ID NO: 40. In some embodiments, the anti-FasL antibody comprises a V_{H} comprising the amino acid sequence of SEQ ID NO: 23 and a V_{L} comprising the amino acid sequence of SEQ ID NO: 40.

In some embodiments, the anti-FasL antibody comprises a V_{H} comprising the amino acid sequence of SEQ ID NO: 24, or a variant thereof having at least about 80% (such as at least about any of 80%, 85%, 90%, 95%, 96%, 97%, 98%, or 99%) sequence identity to the amino acid sequence of SEQ ID NO: 24, and a V_{L} comprising the amino acid sequence of SEQ ID NO: 40, or a variant thereof having at least about 80% (such as at least about any of 80%, 85%, 90%, 95%, 96%, 97%, 98%, or 99%) sequence identity to the amino acid sequence of SEQ ID NO: 40. In some embodiments, the anti-FasL antibody comprises a V_{H} comprising the amino acid sequence of SEQ ID NO: 24 and a V_{L} comprising the amino acid sequence of SEQ ID NO: 40.

In some embodiments, the anti-FasL antibody comprises a V_{H} comprising the amino acid sequence of SEQ ID NO: 25, or a variant thereof having at least about 80% (such as at least about any of 80%, 85%, 90%, 95%, 96%, 97%, 98%, or 99%) sequence identity to the amino acid sequence of SEQ ID NO: 25, and a V_{L} comprising the amino acid sequence of SEQ ID NO: 40, or a variant thereof having at least about 80% (such as at least about any of 80%, 85%, 90%, 95%, 96%, 97%, 98%, or 99%) sequence identity to the amino acid sequence of SEQ ID NO: 40. In some embodiments, the anti-FasL antibody comprises a V_{H} comprising the amino acid sequence of SEQ ID NO: 25 and a V_{L} comprising the amino acid sequence of SEQ ID NO: 40.

In some embodiments, the anti-FasL antibody comprises a V_{H} comprising the amino acid sequence of SEQ ID NO: 21, or a variant thereof having at least about 80% (such as at least about any of 80%, 85%, 90%, 95%, 96%, 97%, 98%, or 99%) sequence identity to the amino acid sequence of SEQ ID NO: 21, and a V_{L} comprising the amino acid sequence of SEQ ID NO: 41, or a variant thereof having at least about 80% (such as at least about any of 80%, 85%, 90%, 95%, 96%, 97%, 98%, or 99%) sequence identity to the amino acid sequence of SEQ ID NO: 41. In some embodiments, the anti-FasL antibody comprises a V_{H} comprising the amino acid sequence of SEQ ID NO: 21 and a V_{L} comprising the amino acid sequence of SEQ ID NO: 41.

In some embodiments, the anti-FasL antibody comprises a V_{H} comprising the amino acid sequence of SEQ ID NO: 22, or a variant thereof having at least about 80% (such as at least about any of 80%, 85%, 90%, 95%, 96%, 97/, 98%, or 99%) sequence identity to the amino acid sequence of SEQ ID NO: 22, and a V_{L} comprising the amino acid sequence of SEQ ID NO: 41, or a variant thereof having at least about 80% (such as at least about any of 80%, 85%, 90%, 95%, 96%, 97%, 98%, or 99%) sequence identity to the amino acid sequence of SEQ ID NO: 41. In some embodiments, the anti-FasL antibody comprises a V_{H} comprising the amino acid sequence of SEQ ID NO: 22 and a V_{L} comprising the amino acid sequence of SEQ ID NO: 41.

In some embodiments, the anti-FasL antibody comprises a V_{H} comprising the amino acid sequence of SEQ ID NO: 23, or a variant thereof having at least about 80% (such as at least about any of 80%, 85%, 90%, 95%, 96%, 97/, 98%, or 99%) sequence identity to the amino acid sequence of SEQ ID NO: 23, and a V_{L} comprising the amino acid sequence of SEQ ID NO: 41, or a variant thereof having at least about 80% (such as at least about any of 80%, 85%, 90%, 95%, 96%, 97%, 98%, or 99%) sequence identity to the amino acid sequence of SEQ ID NO: 41. In some embodiments, the anti-FasL antibody comprises a V_{H} comprising the amino acid sequence of SEQ ID NO: 23 and a V_{L} comprising the amino acid sequence of SEQ ID NO: 41.

In some embodiments, the anti-FasL antibody comprises a V_{H} comprising the amino acid sequence of SEQ ID NO: 24, or a variant thereof having at least about 80% (such as at least about any of 80%, 85%, 90%, 95%, 96%, 97/, 98%, or 99%) sequence identity to the amino acid sequence of SEQ ID NO: 24, and a V_{L} comprising the amino acid sequence of SEQ ID NO: 41, or a variant thereof having at least about 80% (such as at least about any of 80%, 85%, 90%, 95%, 96%, 97%, 98%, or 99%) sequence identity to the amino acid sequence of SEQ ID NO: 41. In some embodiments, the anti-FasL antibody comprises a V_{H} comprising the amino acid sequence of SEQ ID NO: 24 and a V_{L} comprising the amino acid sequence of SEQ ID NO: 41.

In some embodiments, the anti-FasL antibody comprises a V_{H} comprising the amino acid sequence of SEQ ID NO: 25, or a variant thereof having at least about 80% (such as at least about any of 80%, 85%, 90%, 95%, 96%, 97/, 98%, or 99%) sequence identity to the amino acid sequence of SEQ ID NO: 25, and a V_{L} comprising the amino acid sequence of SEQ ID NO: 41, or a variant thereof having at least about 80% (such as at least about any of 80%, 85%, 90%, 95%, 96%, 97%, 98%, or 99%) sequence identity to the amino acid sequence of SEQ ID NO: 41. In some embodiments, the anti-FasL antibody comprises a V_{H} comprising the amino acid sequence of SEQ ID NO: 25 and a V_{L} comprising the amino acid sequence of SEQ ID NO: 41.

In some embodiments, the anti-FasL antibody comprises a V_{H} comprising the amino acid sequence of SEQ ID NO: 21, or a variant thereof having at least about 80% (such as at least about any of 80%, 85%, 90%, 95%, 96%, 97%, 98%, or 99%) sequence identity to the amino acid sequence of SEQ ID NO: 21, and a V_{L} comprising the amino acid sequence of SEQ ID NO: 42, or a variant thereof having at least about 80% (such as at least about any of 80%, 85%, 90%, 95%, 96%, 97%, 98%, or 99%) sequence identity to the amino acid sequence of SEQ ID NO: 42. In some embodiments, the anti-FasL antibody comprises a V_{H} comprising the amino acid sequence of SEQ ID NO: 21 and a V_{L} comprising the amino acid sequence of SEQ ID NO: 42.

In some embodiments, the anti-FasL antibody comprises a V_{H} comprising the amino acid sequence of SEQ ID NO: 22, or a variant thereof having at least about 80% (such as at least about any of 80%, 85%, 90%, 95%, 96%, 97%, 98%, or 99%) sequence identity to the amino acid sequence of SEQ ID NO: 22, and a V_{L} comprising the amino acid sequence of SEQ ID NO: 42, or a variant thereof having at least about 80% (such as at least about any of 80%, 85%, 90%, 95%, 96%, 97%, 98%, or 99%) sequence identity to the amino acid sequence of SEQ ID NO: 42. In some embodiments, the anti-FasL antibody comprises a V_{H} comprising the amino acid sequence of SEQ ID NO: 22 and a V_{L} comprising the amino acid sequence of SEQ ID NO: 42.

In some embodiments, the anti-FasL antibody comprises a V_{H} comprising the amino acid sequence of SEQ ID NO: 23, or a variant thereof having at least about 80% (such as at least about any of 80%, 85%, 90%, 95%, 96%, 97%, 98%, or 99%) sequence identity to the amino acid sequence of SEQ ID NO: 23, and a V_{L} comprising the amino acid sequence of SEQ ID NO: 42, or a variant thereof having at least about 80% (such as at least about any of 80%, 85%, 90%, 95%, 96%, 97%, 98%, or 99%) sequence identity to the amino acid sequence of SEQ ID NO: 42. In some embodiments, the anti-FasL antibody comprises a V_{H} comprising the amino acid sequence of SEQ ID NO: 23 and a V_{L} comprising the amino acid sequence of SEQ ID NO: 42.

In some embodiments, the anti-FasL antibody comprises a V_{H} comprising the amino acid sequence of SEQ ID NO: 24, or a variant thereof having at least about 80% (such as at least about any of 80%, 85%, 90%, 95%, 96%, 97%, 98%, or 99%) sequence identity to the amino acid sequence of SEQ ID NO: 24, and a V_{L} comprising the amino acid sequence of SEQ ID NO: 42, or a variant thereof having at least about 80% (such as at least about any of 80%, 85%, 90%, 95%, 96%, 97%, 98%, or 99%) sequence identity to the amino acid sequence of SEQ ID NO: 42. In some embodiments, the anti-FasL antibody comprises a V_{H} comprising the amino acid sequence of SEQ ID NO: 24 and a V_{L} comprising the amino acid sequence of SEQ ID NO: 42.

In some embodiments, the anti-FasL antibody comprises a V_{H} comprising the amino acid sequence of SEQ ID NO: 25, or a variant thereof having at least about 80% (such as at least about any of 80%, 85%, 90%, 95%, 96%, 97%, 98%, or 99%) sequence identity to the amino acid sequence of SEQ ID NO: 25, and a V_{L} comprising the amino acid sequence of SEQ ID NO: 42, or a variant thereof having at least about 80% (such as at least about any of 80%, 85%, 90%, 95%, 96%, 97%, 98%, or 99%) sequence identity to the amino acid sequence of SEQ ID NO: 42. In some embodiments, the anti-FasL antibody comprises a V_{H} comprising the amino acid sequence of SEQ ID NO: 25 and a V_{L} comprising the amino acid sequence of SEQ ID NO: 42.

In some embodiments, the anti-FasL antibody comprises a V_{H} comprising: an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8, or a variant thereof comprising up to about 5 amino acid substitutions in the HC-CDRs; and a V_{L} comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17, or a variant thereof comprising up to about 5 amino acid substitutions in the LC-CDRs.

In some embodiments, the anti-FasL antibody comprises a V_{H} comprising: an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8; and a V_{L} comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17.

In some embodiments, the anti-FasL antibody comprises a V_{H} comprising an HC-CDR1, an HC-CDR2 and an HC-CDR3 of the V_{H} comprising the amino acid sequence of SEQ ID NO: 26, and a V_{L} comprising an LC-CDR1, an LC-CDR2 and an LC-CDR3 of the V_{L} comprising the amino acid sequence of SEQ ID NO: 43.

In some embodiments, the anti-FasL antibody comprises a V_{H} comprising the amino acid sequence of any one of SEQ ID NOs: 26-28, or a variant thereof having at least about 80% (such as at least about any of 80%, 85%, 90%, 95%, 96%, 97%, 98%, or 99%) sequence identity to the amino acid sequence of any one of SEQ ID NOs: 26-28, and a V_{L} comprising the amino acid sequence of any one of SEQ ID NOs: 43-45, or a variant thereof having at least about 80% (such as at least about any of 80%, 85%, 90%, 95%, 96%, 97%, 98%, or 99%) sequence identity to the amino acid sequence of any one of SEQ ID NOs: 43-45. In some embodiments, the anti-FasL antibody comprises a V_{H} comprising the amino acid sequence of any one of SEQ ID NOs: 26-28, and a V_{L} comprising the amino acid sequence of any one of SEQ ID NOs: 43-45.

In some embodiments, the anti-FasL antibody comprises a V_{H} comprising the amino acid sequence of SEQ ID NO: 26, or a variant thereof having at least about 80% (such as at least about any of 80%, 85%, 90%, 95%, 96%, 97%, 98%, or 99%) sequence identity to the amino acid sequence of SEQ ID NO: 26, and a V_{L} comprising the amino acid sequence of SEQ ID NO: 43, or a variant thereof having at least about 80% (such as at least about any of 80%, 85%, 90%, 95%, 96%, 97%, 98%, or 99%) sequence identity to the amino acid sequence of SEQ ID NO: 43. In some embodiments, the anti-FasL antibody comprises a V_{H} comprising the amino acid sequence of SEQ ID NO: 26 and a V_{L} comprising the amino acid sequence of SEQ ID NO: 43.

In some embodiments, the anti-FasL antibody comprises a V_{H} comprising the amino acid sequence of SEQ ID NO: 27, or a variant thereof having at least about 80% (such as at least about any of 80%, 85%, 90%, 95%, 96%, 97%, 98%, or 99%) sequence identity to the amino acid sequence of SEQ ID NO: 27, and a V_{L} comprising the amino acid sequence of SEQ ID NO: 44, or a variant thereof having at least about 80% (such as at least about any of 80%, 85%, 90%, 95%, 96%, 97%, 98%, or 99%) sequence identity to the amino acid sequence of SEQ ID NO: 44. In some embodiments, the anti-FasL antibody comprises a V_{H} comprising the amino acid sequence of SEQ ID NO: 27 and a V_{L} comprising the amino acid sequence of SEQ ID NO: 44.

In some embodiments, the anti-FasL antibody comprises a V_{H} comprising the amino acid sequence of SEQ ID NO: 28, or a variant thereof having at least about 80% (such as at least about any of 80%, 85%, 90%, 95%, 96%, 97%, 98%, or 99%) sequence identity to the amino acid sequence of SEQ ID NO: 28, and a V_{L} comprising the amino acid sequence of SEQ ID NO: 44, or a variant thereof having at least about 80% (such as at least about any of 80%, 85%, 90%, 95%, 96%, 97%, 98%, or 99%) sequence identity to the amino acid sequence of SEQ ID NO: 44. In some embodiments, the anti-FasL antibody comprises a V_{H} comprising the amino acid sequence of SEQ ID NO: 28 and a V_{L} comprising the amino acid sequence of SEQ ID NO: 44.

In some embodiments, the anti-FasL antibody comprises a V_{H} comprising the amino acid sequence of SEQ ID NO: 27, or a variant thereof having at least about 80% (such as at least about any of 80%, 85%, 90%, 95%, 96%, 97%, 98%, or 99%) sequence identity to the amino acid sequence of SEQ ID NO: 27, and a V_{L} comprising the amino acid sequence of SEQ ID NO: 45, or a variant thereof having at least about 80% (such as at least about any of 80%, 85%, 90%, 95%, 96%, 97%, 98%, or 99%) sequence identity to the amino acid sequence of SEQ ID NO: 45. In some embodiments, the anti-FasL antibody comprises a V_{H} comprising the amino acid sequence of SEQ ID NO: 27 and a V_{L} comprising the amino acid sequence of SEQ ID NO: 45.

In some embodiments, the anti-FasL antibody comprises a V_{H} comprising the amino acid sequence of SEQ ID NO: 28, or a variant thereof having at least about 80% (such as at least about any of 80%, 85%, 90%, 95%, 96%, 97%, 98%, or 99%) sequence identity to the amino acid sequence of SEQ ID NO: 28, and a V_{L} comprising the amino acid sequence of SEQ ID NO: 45, or a variant thereof having at least about 80% (such as at least about any of 80%, 85%, 90%, 95%, 96%, 97%, 98%, or 99%) sequence identity to the amino acid sequence of SEQ ID NO: 45. In some embodiments, the anti-FasL antibody comprises a V_{H} comprising the amino acid sequence of SEQ ID NO: 28 and a V_{L} comprising the amino acid sequence of SEQ ID NO: 45.

In some embodiments, the anti-FasL antibody comprises a V_{H} comprising: an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9, or a variant thereof comprising up to about 5 amino acid substitutions in the HC-CDRs; and a V_{L} comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18, or a variant thereof comprising up to about 5 amino acid substitutions in the LC-CDRs.

In some embodiments, the anti-FasL antibody comprises a V_{H} comprising: an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9; and a V_{L} comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18.

In some embodiments, the anti-FasL antibody comprises a V_{H} comprising an HC-CDR1, an HC-CDR2 and an HC-CDR3 of the V_{H} comprising the amino acid sequence of SEQ ID NO: 31, and a V_{L} comprising an LC-CDR1, an LC-CDR2 and an LC-CDR3 of the V_{L} comprising the amino acid sequence of SEQ ID NO: 48.

In some embodiments, the anti-FasL antibody comprises a V_{H} comprising the amino acid sequence of any one of SEQ ID NOs: 29-37, or a variant thereof having at least about 80% (such as at least about any of 80%, 85%, 90%, 95%, 96%, 97%, 98%, or 99%) sequence identity to the amino acid sequence of any one of SEQ ID NOs: 29-37, and a V_{L} comprising the amino acid sequence of any one of SEQ ID NOs: 46-52, or a variant thereof having at least about 80% (such as at least about any of 80%, 85%, 90%, 95%, 96%, 97%, 98%, or 99%) sequence identity to the amino acid sequence of any one of SEQ ID NOs: 46-52. In some embodiments, the anti-FasL antibody comprises a V_{H} comprising the amino acid sequence of any one of SEQ ID NOs: 29-37, and a V_{L} comprising the amino acid sequence of any one of SEQ ID NOs: 46-52.

In some embodiments, the anti-FasL antibody comprises a V_{H} comprising the amino acid sequence of SEQ ID NO: 29, or a variant thereof having at least about 80% (such as at least about any of 80%, 85%, 90%, 95%, 96%, 97%, 98%, or 99%) sequence identity to the amino acid sequence of SEQ ID NO: 29, and a V_{L} comprising the amino acid sequence of SEQ ID NO: 46, or a variant thereof having at least about 80% (such as at least about any of 80%, 85%, 90%, 95%, 96%, 97%, 98%, or 99%) sequence identity to the amino acid sequence of SEQ ID NO: 46. In some embodiments, the anti-FasL antibody comprises a V_{H} comprising the amino acid sequence of SEQ ID NO: 29 and a V_{L} comprising the amino acid sequence of SEQ ID NO: 46.

In some embodiments, the anti-FasL antibody comprises a V_{H} comprising the amino acid sequence of SEQ ID NO: 30, or a variant thereof having at least about 80% (such as at least about any of 80%, 85%, 90%, 95%, 96%, 97/, 98%, or 99%) sequence identity to the amino acid sequence of SEQ ID NO: 30, and a V_{L} comprising the amino acid sequence of SEQ ID NO: 47, or a variant thereof having at least about 80% (such as at least about any of 80%, 85%, 90%, 95%, 96%, 97%, 98%, or 99%) sequence identity to the amino acid sequence of SEQ ID NO: 47. In some embodiments, the anti-FasL antibody comprises a V_{H} comprising the amino acid sequence of SEQ ID NO: 30 and a V_{L} comprising the amino acid sequence of SEQ ID NO: 47.

In some embodiments, the anti-FasL antibody comprises a V_{H} comprising the amino acid sequence of SEQ ID NO: 31, or a variant thereof having at least about 80% (such as at least about any of 80%, 85%, 90%, 95%, 96%, 97/, 98%, or 99%) sequence identity to the amino acid sequence of SEQ ID NO: 31, and a V_{L} comprising the amino acid sequence of SEQ ID NO: 48, or a variant thereof having at least about 80% (such as at least about any of 80%, 85%, 90%, 95%, 96%, 97%, 98%, or 99%) sequence identity to the amino acid sequence of SEQ ID NO: 48. In some embodiments, the anti-FasL antibody comprises a V_{H} comprising the amino acid sequence of SEQ ID NO: 31 and a V_{L} comprising the amino acid sequence of SEQ ID NO: 48.

In some embodiments, the anti-FasL antibody comprises a V_{H} comprising the amino acid sequence of SEQ ID NO: 31, or a variant thereof having at least about 80% (such as at least about any of 80%, 85%, 90%, 95%, 96%, 97/, 98%, or 99%) sequence identity to the amino acid sequence of SEQ ID NO: 31, and a V_{L} comprising the amino acid sequence of SEQ ID NO: 49, or a variant thereof having at least about 80% (such as at least about any of 80%, 85%, 90%, 95%, 96%, 97%, 98%, or 99%) sequence identity to the amino acid sequence of SEQ ID NO: 49. In some embodiments, the anti-FasL antibody comprises a V_{H} comprising the amino acid sequence of SEQ ID NO: 31 and a V_{L} comprising the amino acid sequence of SEQ ID NO: 49.

In some embodiments, the anti-FasL antibody comprises a V_{H} comprising the amino acid sequence of SEQ ID NO: 32, or a variant thereof having at least about 80% (such as at least about any of 80%, 85%, 90%, 95%, 96%, 97/, 98%, or 99%) sequence identity to the amino acid sequence of SEQ ID NO: 32, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity to the amino acid sequence of SEQ ID NO: 50. In some embodiments, the anti-FasL antibody comprises a V_{H} comprising the amino acid sequence of SEQ ID NO: 32 and a V_{L} comprising the amino acid sequence of SEQ ID NO: 50.

In some embodiments, the anti-FasL antibody comprises a V_{H} comprising the amino acid sequence of SEQ ID NO: 34, or a variant thereof having at least about 80% (such as at least about any of 80%, 85%, 90%, 95%, 96%, 97%, 98%, or 99%) sequence identity to the amino acid sequence of SEQ ID NO: 34, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity to the amino acid sequence of SEQ ID NO: 50. In some embodiments, the anti-FasL antibody comprises a V_{H} comprising the amino acid sequence of SEQ ID NO: 34 and a V_{L} comprising the amino acid sequence of SEQ ID NO: 50.

In some embodiments, the anti-FasL antibody comprises a V_{H} comprising the amino acid sequence of SEQ ID NO: 35, or a variant thereof having at least about 80% (such as at least about any of 80%, 85%, 90%, 95%, 96%, 97%, 98%, or 99%) sequence identity to the amino acid sequence of SEQ ID NO: 35, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity to the amino acid sequence of SEQ ID NO: 50. In some embodiments, the anti-FasL antibody comprises a V_{H} comprising the amino acid sequence of SEQ ID NO: 35 and a V_{L} comprising the amino acid sequence of SEQ ID NO: 50.

In some embodiments, the anti-FasL antibody comprises a V_{H} comprising the amino acid sequence of SEQ ID NO: 36, or a variant thereof having at least about 80% (such as at least about any of 80%, 85%, 90%, 95%, 96%, 97%, 98%, or 99%) sequence identity to the amino acid sequence of SEQ ID NO: 36, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity to the amino acid sequence of SEQ ID NO: 50. In some embodiments, the anti-FasL antibody comprises a V_{H} comprising the amino acid sequence of SEQ ID NO: 36 and a V_{L} comprising the amino acid sequence of SEQ ID NO: 50.

In some embodiments, the anti-FasL antibody comprises a V_{H} comprising the amino acid sequence of SEQ ID NO: 37, or a variant thereof having at least about 80% (such as at least about any of 80%, 85%, 90%, 95%, 96%, 97/, 98%, or 99%) sequence identity to the amino acid sequence of SEQ ID NO: 37, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity to the amino acid sequence of SEQ ID NO: 50. In some embodiments, the anti-FasL antibody comprises a V_{H} comprising the amino acid sequence of SEQ ID NO: 37 and a V_{L} comprising the amino acid sequence of SEQ ID NO: 50.

In some embodiments, the anti-FasL antibody comprises a V_{H} comprising the amino acid sequence of SEQ ID NO: 32, or a variant thereof having at least about 80% (such as at least about any of 80%, 85%, 90%, 95%, 96%, 97/, 98%, or 99%) sequence identity to the amino acid sequence of SEQ ID NO: 32, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 32 and a V_{L} comprising the amino acid sequence of SEQ ID NO: 51.

In some embodiments, the anti-FasL antibody comprises a V_{H} comprising the amino acid sequence of SEQ ID NO: 33, or a variant thereof having at least about 80% (such as at least about any of 80%, 85%, 90%, 95%, 96%, 97/, 98%, or 99%) sequence identity to the amino acid sequence of SEQ ID NO: 33, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 33 and a V_{L} comprising the amino acid sequence of SEQ ID NO: 51.

In some embodiments, the anti-FasL antibody comprises a V_{H} comprising the amino acid sequence of SEQ ID NO: 34, or a variant thereof having at least about 80% (such as at least about any of 80%, 85%, 90%, 95%, 96%, 97/, 98%, or 99%) sequence identity to the amino acid sequence of SEQ ID NO: 34, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 34 and a V_{L} comprising the amino acid sequence of SEQ ID NO: 51.

In some embodiments, the anti-FasL antibody comprises a V_{H} comprising the amino acid sequence of SEQ ID NO: 35, or a variant thereof having at least about 80% (such as at least about any of 80%, 85%, 90%, 95%, 96%, 97%, 98%, or 99%) sequence identity to the amino acid sequence of SEQ ID NO: 35, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 35 and a V_{L} comprising the amino acid sequence of SEQ ID NO: 51.

In some embodiments, the anti-FasL antibody comprises a V_{H} comprising the amino acid sequence of SEQ ID NO: 36, or a variant thereof having at least about 80% (such as at least about any of 80%, 85%, 90%, 95%, 96%, 97%, 98%, or 99%) sequence identity to the amino acid sequence of SEQ ID NO: 36, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 36 and a V_{L} comprising the amino acid sequence of SEQ ID NO: 51.

In some embodiments, the anti-FasL antibody comprises a V_{H} comprising the amino acid sequence of SEQ ID NO: 37, or a variant thereof having at least about 80% (such as at least about any of 80%, 85%, 90%, 95%, 96%, 97%, 98%, or 99%) sequence identity to the amino acid sequence of SEQ ID NO: 37, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity to the amino acid sequence of SEQ ID NO: 51. In some embodiments, the anti-FasL antibody comprises a V_{H} comprising the amino acid sequence of SEQ ID NO: 37 and a V_{L} comprising the amino acid sequence of SEQ ID NO: 51.

In some embodiments, the anti-FasL antibody comprises a V_{H} comprising the amino acid sequence of SEQ ID NO: 32, or a variant thereof having at least about 80% (such as at least about any of 80%, 85%, 90%, 95%, 96%, 97%, 98%, or 99%) sequence identity to the amino acid sequence of SEQ ID NO: 32, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity to the amino acid sequence of SEQ ID NO: 52. In some embodiments, the anti-FasL antibody comprises a V_{H} comprising the amino acid sequence of SEQ ID NO: 32 and a V_{L} comprising the amino acid sequence of SEQ ID NO: 52.

In some embodiments, the anti-FasL antibody comprises a V_{H} comprising the amino acid sequence of SEQ ID NO: 34, or a variant thereof having at least about 80% (such as at least about any of 80%, 85%, 90%, 95%, 96%, 97/, 98%, or 99%) sequence identity to the amino acid sequence of SEQ ID NO: 34, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity to the amino acid sequence of SEQ ID NO: 52. In some embodiments, the anti-FasL antibody comprises a V_{H} comprising the amino acid sequence of SEQ ID NO: 34 and a V_{L} comprising the amino acid sequence of SEQ ID NO: 52.

In some embodiments, the anti-FasL antibody comprises a V_{H} comprising the amino acid sequence of SEQ ID NO: 35, or a variant thereof having at least about 80% (such as at least about any of 80%, 85%, 90%, 95%, 96%, 97/, 98%, or 99%) sequence identity to the amino acid sequence of SEQ ID NO: 35, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity to the amino acid sequence of SEQ ID NO: 52. In some embodiments, the anti-FasL antibody comprises a V_{H} comprising the amino acid sequence of SEQ ID NO: 35 and a V_{L} comprising the amino acid sequence of SEQ ID NO: 52.

In some embodiments, the anti-FasL antibody comprises a V_{H} comprising the amino acid sequence of SEQ ID NO: 36, or a variant thereof having at least about 80% (such as at least about any of 80%, 85%, 90%, 95%, 96%, 97/, 98%, or 99%) sequence identity to the amino acid sequence of SEQ ID NO: 36, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity to the amino acid sequence of SEQ ID NO: 52. In some embodiments, the anti-FasL antibody comprises a V_{H} comprising the amino acid sequence of SEQ ID NO: 36 and a V_{L} comprising the amino acid sequence of SEQ ID NO: 52.

In some embodiments, the anti-FasL antibody comprises a V_{H} comprising the amino acid sequence of SEQ ID NO: 37, or a variant thereof having at least about 80% (such as at least about any of 80%, 85%, 90%, 95%, 96%, 97%, 98%, or 99%) sequence identity to the amino acid sequence of SEQ ID NO: 37, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity to the amino acid sequence of SEQ ID NO: 52. In some embodiments, the anti-FasL antibody comprises a V_{H} comprising the amino acid sequence of SEQ ID NO: 37 and a V_{L} comprising the amino acid sequence of SEQ ID NO: 52.

In some embodiments, the amino acid substitutions described above are limited to “exemplary substitutions” shown in Table 4 of this application. In some embodiments, the amino acid substitutions are limited to “preferred substitutions” shown in Table 4 of this application.

In some embodiments, functional epitopes can be mapped by combinatorial alanine scanning. In this process, a combinatorial alanine-scanning strategy can be used to identify amino acids in the FasL protein that are necessary for interaction with FasL antibodies. In some embodiments, the epitope is conformational and crystal structure of anti-FasL antibodies bound to FasL may be employed to identify the epitopes.

In some embodiments, the present application provides antibodies which compete with any one of the FasL antibodies described herein for binding to FasL. In some embodiments, the present application provides antibodies which compete with any one of the anti-FasL antibodies provided herein for binding to an epitope on the FasL. In some embodiments, an anti-FasL antibody is provided that binds to the same epitope as an anti-FasL antibody comprising a V_{H} comprising the amino acid sequence of any one of SEQ ID NOs: 19-37, and a V_{L} comprising the amino acid sequence of any one of SEQ ID NOs: 38-52. In some embodiments, an anti-FasL antibody is provided that specifically binds to FasL competitively with an anti-FasL antibody comprising a V_{H} comprising the amino acid sequence of any one of SEQ ID NOs: 19-37 and a V_{L} comprising the amino acid sequence of any one of SEQ ID NOs: 38-52.

In some embodiments, competition assays may be used to identify a monoclonal antibody that competes with an anti-FasL antibody described herein for binding to FasL. Competition assays can be used to determine whether two antibodies bind to the same epitope by recognizing identical or sterically overlapping epitopes or one antibody competitively inhibits binding of another antibody to the antigen. In certain embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, N.Y.). Detailed exemplary methods for mapping an epitope to which an antibody binds are provided in Morris (1996) “Epitope Mapping Protocols”, in Methods in Molecular Biology vol. 66 (Humana Press, Totowa, N.J.). In some embodiments, two antibodies are said to bind to the same epitope if each blocks binding of the other by 50% or more. In some embodiments, the antibody that competes with an anti-FasL antibody described herein is a chimeric, humanized or human antibody.

Exemplary anti-FasL antibody sequences are shown in Tables 2 and 3, wherein the CDR numbering is according to Chothia definition. Those skilled in the art will recognize that many algorithms (as defined by Chothia) are known for prediction of CDR positions and for delimitation of antibody heavy chain and light chain variable regions. Anti-FasL antibodies comprising CDRs, V_{H} and/or V_{L} sequences from antibodies described herein, but based on prediction algorithms other than those exemplified in the tables below, are within the scope of this invention.

TABLE 2
Exemplary anti-FasL antibody CDR sequences.

| Antibody Name | HC-CDR1 | HC-CDR2 | HC-CDR3 |
|---|---|---|---|
| FL-M54 | GYTFTRF | PGDG | LISTAMFD |
| FL-M56 | (SEQ ID NO: 1) | (SEQ ID NO: 4) | (SEQ ID NO: 7) |
| humFL-M54-1 | | | |
| humFL-M54-2 | | | |
| humFL-M54-3 | | | |
| humFL-M54-4 | | | |
| humFL-M54-5 | | | |
| humFL-M54-6 | | | |
| humFL-M54-7 | | | |
| humFL-M54-8 | | | |
| humFL-M54-9 | | | |
| humFL-M54-10 | | | |
| humFL-M54-11 | | | |
| humFL-M54-12 | | | |
| humFL-M54-13 | | | |
| humFL-M54-14 | | | |
| humFL-M54-15 | | | |
| FL-M78 | GFTFSSY | SGGG | YDNYLYAMD |
| humFL-M78-1 | (SEQ ID NO: 2) | (SEQ ID NO: 5) | (SEQ ID NO: 8) |
| humFL-M78-2 | | | |
| humFL-M78-3 | | | |
| humFL-M78-4 | | | |
| FL-M05 | GYTFTDY | TYQG | PDWDYAMD |
| FL-M08 | (SEQ ID NO: 3) | (SEQ ID NO: 6) | (SEQ ID NO: 9) |
| FL-M88 | | | |
| FL-M89 | | | |
| humFL-M88-1 | | | |
| humFL-M88-2 | | | |
| humFL-M88-3 | | | |
| humFL-M88-4 | | | |
| humFL-M88-5 | | | |
| humFL-M88-6 | | | |
| humFL-M88-7 | | | |
| humFL-M88-8 | | | |
| humFL-M88-9 | | | |
| humFL-M88-10 | | | |
| humFL-M88-11 | | | |
| humFL-M88-12 | | | |
| humFL-M88-13 | | | |
| humFL-M88-14 | | | |
| humFL-M88-15 | | | |
| humFL-M88-16 | | | |
| humFL-M88-17 | | | |
| humFL-M88-18 | | | |
| FL-M54 | SKSVSTSDFSY | LAS | SRELPR |
| FL-M56 | (SEQ ID NO: 10) | (SEQ ID NO: 13) | (SEQ ID NO: 16) |
| humFL-M54-1 | | | |
| humFL-M54-2 | | | |
| humFL-M54-3 | | | |
| humFL-M54-4 | | | |
| humFL-M54-5 | | | |
| humFL-M54-6 | | | |
| humFL-M54-7 | | | |
| humFL-M54-8 | | | |
| humFL-M54-9 | | | |
| humFL-M54-10 | | | |
| humFL-M54-11 | | | |
| humFL-M54-12 | | | |
| humFL-M54-13 | | | |
| humFL-M54-14 | | | |
| humFL-M54-15 | | | |
| FL-M78 | SQTIGTW | AAT | LYSTPF |
| humFL-M78-1 | (SEQ ID NO: 11) | (SEQ ID NO: 14) | (SEQ ID NO: 17) |
| humFL-M78-2 | | | |
| humFL-M78-3 | | | |
| humFL-M78-4 | | | |
| FL-M05 | | | |
| FL-M08 | | | |
| FL-M88 | | | |
| FL-M89 | | | |
| humFL-M88-1 | SQSVDYDGDSY | AAS | SRELPY |
| humFL-M88-2 | (SEQ ID NO: 12) | (SEQ ID NO: 15) | (SEQ ID NO: 18) |
| humFL-M88-3 | | | |
| humFL-M88-4 | | | |
| humFL-M88-5 | | | |
| humFL-M88-6 | | | |
| humFL-M88-7 | | | |
| humFL-M88-8 | | | |
| humFL-M88-9 | | | |
| humFL-M88-10 | | | |
| humFL-M88-11 | | | |
| humFL-M88-12 | | | |
| humFL-M88-13 | | | |
| humFL-M88-14 | | | |
| humFL-M88-15 | | | |
| humFL-M88-16 | | | |
| humFL-M88-17 | | | |
| humFL-M88-18 | | | |

TABLE 3
Exemplary sequences.

| SEQ ID | | |
|---|---|---|
| NO | Description | Sequence |
| 19 | FL-M54 V_{H} | EVQLQQSGVELVKPGASVKLSCKASGYTFTRFDISWVRQRPEQ |
| | | GLEWIGWIFPGDGSSQYNENFKGKATLTTDKSSGTAYMQLNR |
| | | LTSEDSAVYFCARSLISTAMFDHWGQGTTLTVSS |
| 20 | FL-M56 V_{H} | EVQLQQSGPELVKPGASVKLSCKASGYTFTRFDISWVRQRPEQ |
| | | GLEWIGWIFPGDGSTQYNENFKGKATLTTDKSSGTAYMQLNR |
| | | LTSEDSAVYFCARSLISTAMFDHWGQGTTLTVSS |
| 21 | humFL-M54-1 V_{H} | QVQLVQSGAEVKKPGSSVKVSCKASGYTFTRFDISWVRQAPG |
| | humFL-M54-6 V_{H} | QGLEWIGWIFPGDGSSQYNENFKGRATLTTDKSTSTAYMELSS |
| | humFL-M54-11 V_{H} | LRSEDTAVYFCARSLISTAMFDHWGQGTLVTVSS |
| 22 | humFL-M54-2 V_{H} | QVQLVQSGAEVKKPGSSVKVSCKASGYTFTRFDISWVRQAPG |
| | humFL-M54-7 V_{H} | QGLEWMGWIFPGDGSSQYNENFKGRATLTTDKSTSTAYMELS |
| | | SLRSEDTAVYFCARSLISTAMFDHWGQGTLVTVSS |
| | humFL-M54-12 V_{H} | |
| 23 | humFL-M54-3 V_{H} | QVQLVQSGAEVKKPGSSVKVSCKASGYTFTRFDISWVRQAPG |
| | humFL-M54-8 V_{H} | QGLEWMGWIFPGDGSSQYNENFKGRATITTDKSTSTAYMELS |
| | humFL-M54-13 V_{H} | SLRSEDTAVYFCARSLISTAMFDHWGQGTLVTVSS |
| 24 | humFL-M54-4 V_{H} | QVQLVQSGAEVKKPGSSVKVSCKASGYTFTRFDISWVRQAPG |
| | humFL-M54-9 V_{H} | QGLEWMGWIFPGDGSSQYNENFKGRVTLTTDKSTSTAYMELS |
| | humFL-M54-14 V_{H} | SLRSEDTAVYFCARSLISTAMFDHWGQGTLVTVSS |
| 25 | humFL-M54-5 V_{H} | QVQLVQSGAEVKKPGSSVKVSCKASGYTFTRFDISWVRQAPG |
| | humFL-M54-10 V_{H} | QGLEWMGWIFPGDGSSQYNENFKGRVTITADKSTSTAYMELS |
| | humFL-M54-15 V_{H} | SLRSEDTAVYFCARSLISTAMFDHWGQGTLVTVSS |
| 26 | FL-M78 V_{H} | DVQLQESGGGLVQPGGSRKLSCAASGFTFSSYDMSWVRQTPE |
| | | KRLEWVAFISSGGGSTYYPDTVKGRFTISRDNAKNTLYLQMSS |
| | | LKSEDSAMYYCGRHYDNYLYAMDYWGQGTSVTVSS |
| 27 | humFL-M78-1 V_{H} | EVQLVESGGGLVQPGGSLRLSCAASGFTFSSYDMSWVRQAPG |
| | humFL-M78-3 V_{H} | KRLEWVAFISSGGGSTYYPDTVKGRFTISRDNAKNSLYLQMNS |
| | | LRAEDTAVYYCGRHYDNYLYAMDYWGQGTLVTVSS |
| 28 | humFL-M78-2 V_{H} | EVQLVESGGGLVQPGGSLRLSCAASGFTFSSYDMSWVRQAPG |
| | humFL-M78-4 V_{H} | KGLEWVAFISSGGGSTYYPDTVKGRFTISRDNAKNSLYLQMNS |
| | | LRAEDTAVYYCGRHYDNYLYAMDYWGQGTLVTVSS |
| 29 | FL-M05 V_{H} | EVQLQQSGAELVRPGVSVKISCKGSGYTFTDYTMHWVKQSHA |
| | | KSLEWIGVISTYQGDARYNQKFKGKATMTVDKSSSTAYMELA |
| | | RLTSEDSAIYYCARFPDWDYAMDYWGQGTSVTVSS |
| 30 | FL-M08 V_{H} | QIQLLQSGAELVRPGVSVKISCKGSGYTFTDYAMHWVKQSHA |
| | | KSLEWIGVISTYQGDANYNQKFKGKATMTVDKSSSTAYMELA |
| | | RLTSEDSAIYYCARFPDWDYAMDYWGQGTSVTVSS |
| 31 | FL-M88 V_{H} | EVQLQQSGAELVRPGVSVKISCKGSGYTFTDYTIHWVKQSHA |
| | FL-M89 V_{H} | KSLEWIGVISTYQGDASYNQKFKGKATMTVDKSSSTAYMELA |
| | | RLTSEDSAIYYCARFPDWDYAMDYWGQGTSVTVSS |
| 32 | humFL-M88-1 V_{H} | QVQLVQSGAEVKKPGASVKVSCKGSGYTFTDYTIHWVRQAP |
| | humFL-M88-7 V_{H} | GQGLEWIGVISTYQGDASYNQKFKGRATMTVDKSTSTAYMEL |
| | humFL-M88-13 V_{H} | SSLRSEDTAVYYCARFPDWDYAMDYWGQGTLVTVSS |
| 33 | humFL-M88-2 V_{H} | QVQLVQSGAEVKKPGASVKVSCKASGYTFTDYTIHWVRQAP |
| | humFL-M88-8 V_{H} | GQGLEWIGVISTYQGDASYNQKFKGRATMTVDKSTSTAYMEL |
| | humFL-M88-14 V_{H} | SSLRSEDTAVYYCARFPDWDYAMDYWGQGTLVTVSS |
| 34 | humFL-M88-3 V_{H} | QVQLVQSGAEVKKPGASVKVSCKGSGYTFTDYTIHWVRQAP |
| | humFL-M88-9 V_{H} | GQGLEWMGVISTYQGDASYNQKFKGRATMTVDKSTSTAYME |
| | humFL-M88-15 V_{H} | LSSLRSEDTAVYYCARFPDWDYAMDYWGQGTLVTVSS |
| 35 | humFL-M88-4 V_{H} | QVQLVQSGAEVKKPGASVKVSCKGSGYTFTDYTIHWVRQAP |
| | humFL-M88-10 V_{H} | GQGLEWMGVISTYQGDASYNQKFKGRVTMTVDKSTSTAYME |
| | humFL-M88-16 V_{H} | LSSLRSEDTAVYYCARFPDWDYAMDYWGQGTLVTVSS |
| 36 | humFL-M88-5 V_{H} | QVQLVQSGAEVKKPGASVKVSCKGSGYTFTDYTIHWVRQAP |
| | humFL-M88-11 V_{H} | GQGLEWMGVISTYQGDASYNQKFKGRVTMTVDKSTSTVYME |
| | humFL-M88-17 V_{H} | LSSLRSEDTAVYYCARFPDWDYAMDYWGQGTLVTVSS |
| 37 | humFL-M88-6 V_{H} | QVQLVQSGAEVKKPGASVKVSCKASGYTFTDYTIHWVRQAP |
| | humFL-M88-12 V_{H} | GQGLEWMGVISTYQGDASYNQKFKGRVTMTVDTSTSTVYME |
| | humFL-M88-18 V_{H} | LSSLRSEDTAVYYCARFPDWDYAMDYWGQGTLVTVSS |
| 38 | FL-M54 V_{L} | DIVMTQSPASLAVSLGQRATISCRASKSVSTSDFSYIHWYQQKP |
| | | GQPPKLLIFLASHLESGVPARFSGSGSGTDFTLNIHPVEEEDAA |
| | | TYYCQHSRELPRTFGGGTKLEIK |
| 39 | FL-M56 V_{L} | DIVLTQSPASLAVSLGQRATISCRASKSVSTSDFSYIHWYQQKP |
| | | GLPPKLLIFLASHLESGVPARFSGSGSGTDFTLNIHPVEEEDAAT |
| | | YYCQHSRELPRTFGGGTKLEIK |
| 40 | humFL-M54-1 V_{L} | EIVMTQSPATLSVSPGERATLSCRASKSVSTSDFSYIHWYQQKP |
| | humFL-M54-2 V_{L} | GQPPRLLIFLASHLESGIPARFSGSGSGTEFTLTISSLQSEDFAVY |
| | humFL-M54-3 V_{L} | YCQHSRELPRTFGQGTKVEIK |
| | humFL-M54-4 V_{L} | |
| | humFL-M54-5 V_{L} | |
| 41 | humFL-M54-6 V_{L} | EIVMTQSPATLSVSPGERATLSCRASKSVSTSDFSYIHWYQQKP |
| | humFL-M54-7 V_{L} | GQPPRLLIYLASHLESGIPARFSGSGSGTEFTLTISSLQSEDFAVY |
| | humFL-M54-8 V_{L} | YCQHSRELPRTFGQGTKVEIK |
| | humFL-M54-9 V_{L} | |
| | humFL-M54-10 V_{L} | |
| 42 | humFL-M54-11 V_{L} | EIVMTQSPATLSVSPGERATLSCRASKSVSTSDFSYIHWYQQKP |
| | humFL-M54-12 V_{L} | GQAPRLLIYLASHLESGIPARFSGSGSGTEFTLTISSLQSEDFAV |
| | humFL-M54-13 V_{L} | YYCQHSRELPRTFGQGTKVEIK |
| | humFL-M54-14 V_{L} | |
| | humFL-M54-15 V_{L} | |
| 43 | FL-M78 V_{L} | DIVMTQSPASQSASLGESVTITCLASQTIGTWLAWYQQKPGKS |
| | | PQLLIYAATSLADGVPSRFSGSGSGTKFSFKISSLQAEDFVSYY |
| | | CQQLYSTPFTFGGGTKLEIK |
| 44 | humFL-M78-1 V_{L} | DIQMTQSPSSLSASVGDRVTITCLASQTIGTWLAWYQQKPGKS |
| | humFL-M78-2 V_{L} | PKLLIYAATSLADGVPSRFSGSGSGTDFTLTISSLQPEDFATYYC |
| | | QQLYSTPFTFGQGTKVEIK |
| 45 | humFL-M78-3 V_{L} | DIQMTQSPSSLSASVGDRVTITCLASQTIGTWLAWYQQKPGKA |
| | humFL-M78-4 V_{L} | PKLLIYAATSLADGVPSRFSGSGSGTDFTLTISSLQPEDFATYYC |
| | | QQLYSTPFTFGQGTKVEIK |
| 46 | FL-M05 V_{L} | DIVLTQSPASLAVSLGQRATISCKASQSVDYDGDSYMNWYQQ |
| | | KPGQPPKLLIYAASNLESGIPARFSGSGSGTDFTLNIHPVEEEDA |
| | | ATYYCQHSRELPYTFGGGTKLEIK |
| 47 | FL-M08 V_{L} | EIVLTQSPASLAVSLGQRATISCKASQSVDYDGDSYMNWYQQ |
| | | KPGQPPKLLIYAASNLESGIPARFSGSGSGTDFTLNIHPVEEEDA |
| | | ATYYCQHSRELPYTFGGGTKLEIK |
| 48 | FL-M88 V_{L} | EIVLTQSPASLAVSLGQRATISCKASQSVDYDGDSYMNWYQQ |
| | | KPGQPPKLLIYAASNLESGVPARFSGSGSGTDFTLNIHPVEEED |
| | | AATYYCQHSRELPYTFGGGTKLEIK |
| 49 | FL-M89 V_{L} | DIVMTQSPASLAVSLGQRATISCKASQSVDYDGDSYMNWYQQ |
| | | KPGQPPKLLIYAASNLESGIPARFSGSGSGTDFTLNIHPVEEEDA |
| | | ATYYCQQSRELPYTFGGGTKLEIK |
| 50 | humFL-M88-1 V_{L} | EIVMTQSPATLSVSPGERATLSCKASQSVDYDGDSYMNWYQQ |
| | humFL-M88-2 V_{L} | KPGQPPRLLIYAASNLESGIPARFSGSGSGTEFTLTISSLQSEDFA |
| | humFL-M88-3 V_{L} | VYYCQHSRELPYTFGQGTKLEIK |
| | humFL-M88-4 V_{L} | |
| | humFL-M88-5 V_{L} | |
| | humFL-M88-6 V_{L} | |
| 51 | humFL-M88-7 V_{L} | EIVMTQSPATLSVSPGERATLSCKASQSVDYDGDSYMNWYQQ |
| | humFL-M88-8 V_{L} | KPGQAPRLLIYAASNLESGIPARFSGSGSGTEFTLTISSLQSEDF |
| | humFL-M88-9 V_{L} | AVYYCQHSRELPYTFGQGTKLEIK |
| | humFL-M88-10 V_{L} | |
| | humFL-M88-11 V_{L} | |
| | humFL-M88-12 V_{L} | |
| 52 | humFL-M88-13 V_{L} | EIVMTQSPATLSVSPGERATLSCKASQSVDYDGDSYMNWYQQ |
| | humFL-M88-14 V_{L} | KPGQPPRLLIYAASNLESGIPARFSGSGSGTEFTLTISSLQSEDFA |
| | humFL-M88-15 V_{L} | VYYCQQSRELPYTFGQGTKLEIK |
| | humFL-M88-16 V_{L} | |
| | humFL-M88-17 V_{L} | |
| | humFL-M88-18 V_{L} | |
| 53 | IgG1 heavy chain | ASTKGPSVFPLAPSSKSTSGGTAALGCLVKDYFPEPVTVSWNS |
| | constant region | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSREEMTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPGK |
| 54 | IgG4 heavy chain | ASTKGPSVFPLAPCSRSTSESTAALGCLVKDYFPEPVTVSWNS |
| | constant region | GALTSGVHTFPAVLQSSGLYSLSSVVTVPSSSLGTKTYTCNVD |
| | | HKPSNTKVDKRVESKYGPPCPSCPAPEFLGGPSVFLFPPKPKDT |
| | | LMISRTPEVTCVVVDVSQEDPEVQFNWYVDGVEVHNAKTKPR |
| | | EEQFNSTYRVVSVLTVLHQDWLNGKEYKCKVSNKGLPSSIEK |
| | | TISKAKGQPREPQVYTLPPSQEEMTKNQVSLTCLVKGFYPSDIA |
| | | VEWESNGQPENNYKTTPPVLDSDGSFFLYSRLTVDKSRWQEG |
| | | NVFSCSVMHEALHNHYTQKSLSLSLGK |
| 55 | Light chain constant | RTVAAPSVFIFPPSDEQLKSGTASVVCLLNNFYPREAKVQWKV |
| | region(kappa) | DNALQSGNSQESVTEQDSKDSTYSLSSTLTLSKADYEKHKVY |
| | | ACEVTHQGLSSPVTKSFNRGEC |
| 56 | Light chain constant | GQPKAAPSVTLFPPSSEELQANKATLVCLISDFYPGAVTVAWK |
| | region(lambda) | ADSSPVKAGVETTTPSKQSNNKYAASSYLSLTPEQWKSHRSYS |
| | | CQVTHEGSTVEKTVAPTECS |
| 57 | human FasL | MQQPFNYPYPQIYWVDSSASSPWAPPGTVLPCPTSVPRRPGQR |
| | | RPPPPPPPPPLPPPPPPPPLPPLPLPPLKKRGNHSTGLCLLVMFFM |
| | | VLVALVGLGLGMFQLFHLQKELAELRESTSQMHTASSLEKQIG |
| | | HPSPPPEKKELRKVAHLTGKSNSRSMPLEWEDTYGIVLLSGVK |
| | | YKKGGLVINETGLYFVYSKVYFRGQSCNNLPLSHKVYMRNSK |
| | | YPQDLVMMEGKMMSYCTTGQMWARSSYLGAVENLTSADHL |
| | | YVNVSELSLVNFEESQTFFGLYKL |
| 58 | human FasL | PSPPPEKKELRKVAHLTGKSNSRSMPLEWEDTYGIVLLSGVKY |
| | extracellular region | KKGGLVINETGLYFVYSKVYFRGQSCNNLPLSHKVYMRNSKY |
| | | PQDLVMMEGKMMSYCTTGQMWARSSYLGAVFNLTSADHLY |
| | | VNVSELSLVNFEESQTFFGLYKL |

### Full-Length Anti-FasL Antibody

The anti-FasL antibody in some embodiments is a full-length anti-FasL antibody. In some embodiments, the full-length anti-FasL antibody is an IgA, IgD, IgE, IgG, or IgM. In some embodiments, the full-length anti-FasL antibody comprises IgG constant domains, such as constant domains of any one of IgG1, IgG2, IgG3, and IgG4 including variants thereof. In some embodiments, the full-length anti-FasL antibody comprises a lambda light chain constant region. In some embodiments, the full-length anti-FasL antibody comprises a kappa light chain constant region. In some embodiments, the full-length anti-FasL antibody is a full-length human anti-FasL antibody. In some embodiments, the full-length anti-FasL antibody comprises an Fc sequence of a mouse immunoglobulin. In some embodiments, the full-length anti-FasL antibody comprises an Fc sequence that has been altered or otherwise changed so that it has enhanced antibody dependent cellular cytotoxicity (ADCC) and complement dependent cytotoxicity (CDC) effector function.

Thus, for example, in some embodiments, there is provided a full-length anti-FasL antibody comprising IgG1 constant domains, wherein the anti-FasL antibody specifically binds to FasL. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG2 constant domains, wherein the anti-FasL antibody specifically binds to FasL. In some embodiments, the IgG2 is human IgG2. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a full-length anti-FasL antibody comprising IgG3 constant domains, wherein the anti-FasL antibody specifically binds to FasL. In some embodiments, the IgG3 is human IgG3. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a full-length anti-FasL antibody comprising IgG4 constant domains, wherein the anti-FasL antibody specifically binds to FasL. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55.

In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a) a heavy chain variable domain comprising an HC-CDR1 comprising the amino acid sequence of any one of SEQ ID NOs: 1-3, an HC-CDR2 comprising the amino acid sequence of any one of SEQ ID NOs: 4-6, and an HC-CDR3 comprising the amino acid sequence of any one of SEQ ID NOs: 7-9, or a variant thereof comprising up to about 5 (such as about any of 1, 2, 3, 4, or 5) amino acid substitutions in the HC-CDR sequences; and b) a light chain variable domain comprising an LC-CDR1 comprising the amino acid sequence of any one of SEQ ID NOs: 10-12, an LC-CDR2 comprising the amino acid sequence of any one of SEQ ID NOs: 13-15, and an LC-CDR3 comprising the amino acid sequence of any one of SEQ ID NOs: 16-18, or a variant thereof comprising up to about 5 (such as about any of 1, 2, 3, 4, or 5) amino acid substitutions in the LC-CDR sequences. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55.

In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a) a heavy chain variable domain comprising an HC-CDR1 comprising the amino acid sequence of any one of SEQ ID NOs: 1-3, an HC-CDR2 comprising the amino acid sequence of any one of SEQ ID NOs: 4-6, and an HC-CDR3 comprising the amino acid sequence of any one of SEQ ID NOs: 7-9, or a variant thereof comprising up to about 5 (such as about any of 1, 2, 3, 4, or 5) amino acid substitutions in the HC-CDR sequences; and b) a light chain variable domain comprising an LC-CDR1 comprising the amino acid sequence of any one of SEQ ID NOs: 10-12, an LC-CDR2 comprising the amino acid sequence of any one of SEQ ID NOs: 13-15, and an LC-CDR3 comprising the amino acid sequence of any one of SEQ ID NOs: 16-18, or a variant thereof comprising up to about 5 (such as about any of 1, 2, 3, 4, or 5) amino acid substitutions in the LC-CDR sequences. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a) a heavy chain variable domain comprising an HC-CDR1 comprising the amino acid sequence of any one of SEQ ID NOs: 1-3, an HC-CDR2 comprising the amino acid sequence of any one of SEQ ID NOs: 4-6, and an HC-CDR3 comprising the amino acid sequence of any one of SEQ ID NOs: 7-9; and b) a light chain variable domain comprising an LC-CDR1 comprising the amino acid sequence of any one of SEQ ID NOs: 10-12, an LC-CDR2 comprising the amino acid sequence of any one of SEQ ID NOs: 13-15, and an LC-CDR3 comprising the amino acid sequence of any one of SEQ ID NOs: 16-18. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a) a heavy chain variable domain comprising an HC-CDR1 comprising the amino acid sequence of any one of SEQ ID NOs: 1-3, an HC-CDR2 comprising the amino acid sequence of any one of SEQ ID NOs: 4-6, and an HC-CDR3 comprising the amino acid sequence of any one of SEQ ID NOs: 7-9; and b) a light chain variable domain comprising an LC-CDR1 comprising the amino acid sequence of any one of SEQ ID NOs: 10-12, an LC-CDR2 comprising the amino acid sequence of any one of SEQ ID NOs: 13-15, and an LC-CDR3 comprising the amino acid sequence of any one of SEQ ID NOs: 16-18. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7; and b) a light chain variable domain comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a) a heavy chain variable domain comprising an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8; and b) a light chain variable domain comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a) a heavy chain variable domain comprising an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9; and b) a light chain variable domain comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7; and b) a light chain variable domain comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a) a heavy chain variable domain comprising an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8; and b) a light chain variable domain comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a) a heavy chain variable domain comprising an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9; and b) a light chain variable domain comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of any one of SEQ ID NOs: 19-37, or a variant thereof having at least about 80% (such as at least about any of 80%, 85%, 90%, 95%, 96%, 97%, 98%, or 99%) sequence identity to the amino acid sequence of any one of SEQ ID NOs: 19-37, and a light chain variable domain comprising the amino acid sequence of any one of SEQ ID NOs: 38-52, or a variant thereof having at least about 80% (such as at least about any of 80%, 85%, 90%, 95%, 96%, 97%, 98%, or 99%) sequence identity to the amino acid sequence of any one of SEQ ID NOs: 38-52. In some embodiments, the IgG1 is human IgG1. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG2 constant domains, wherein the anti-FasL antibody comprises a heavy chain variable domain comprising the amino acid sequence of any one of SEQ ID NOs: 19-37, or a variant thereof having at least about 80% (such as at least about any of 80%, 85%, 90%, 95%, 96%, 97%, 98%, or 99%) sequence identity to the amino acid sequence of any one of SEQ ID NOs: 19-37, and a light chain variable domain comprising the amino acid sequence of any one of SEQ ID NOs: 38-52, or a variant thereof having at least about 80% (such as at least about any of 80%, 85%, 90%, 95%, 96%, 97%, 98%, or 99%) sequence identity to the amino acid sequence of any one of SEQ ID NOs: 38-52. In some embodiments, the IgG2 is human IgG2. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a full-length anti-FasL antibody comprising IgG3 constant domains, wherein the anti-FasL antibody comprises a heavy chain variable domain comprising the amino acid sequence of any one of SEQ ID NOs: 19-37, or a variant thereof having at least about 80% (such as at least about any of 80%, 85%, 90%, 95%, 96%, 97%, 98%, or 99%) sequence identity to the amino acid sequence of any one of SEQ ID NOs: 19-37, and a light chain variable domain comprising the amino acid sequence of any one of SEQ ID NOs: 38-52, or a variant thereof having at least about 80% (such as at least about any of 80%, 85%, 90%, 95%, 96%, 97%, 98%, or 99%) sequence identity to the amino acid sequence of any one of SEQ ID NOs: 38-52. In some embodiments, the IgG3 is human IgG3. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of any one of SEQ ID NOs: 19-37, or a variant thereof having at least about 80% (such as at least about any of 80%, 85%, 90%, 95%, 96%, 97%, 98%, or 99%) sequence identity to the amino acid sequence of any one of SEQ ID NOs: 19-37, and a light chain variable domain comprising the amino acid sequence of any one of SEQ ID NOs: 38-52, or a variant thereof having at least about 80% (such as at least about any of 80%, 85%, 90%, 95%, 96%, 97%, 98%, or 99%) sequence identity to the amino acid sequence of any one of SEQ ID NOs: 38-52. In some embodiments, the IgG4 is human IgG4. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of any one of SEQ ID NOs: 19-37, and a light chain variable domain comprising the amino acid sequence of any one of SEQ ID NOs: 38-52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of any one of SEQ ID NOs: 19-37, and a light chain variable domain comprising the amino acid sequence of any one of SEQ ID NOs: 38-52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 19, or a variant thereof having at least about 80% sequence identity to the amino acid sequence of SEQ ID NO: 19, and a light chain variable domain comprising the amino acid sequence of SEQ ID NO: 38, or a variant thereof having at least about 80% sequence identity to the amino acid sequence of SEQ ID NO: 38. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 20, or a variant thereof having at least about 80% sequence identity to the amino acid sequence of SEQ ID NO: 20, and a light chain variable domain comprising the amino acid sequence of SEQ ID NO: 39, or a variant thereof having at least about 80% sequence identity to the amino acid sequence of SEQ ID NO: 39. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 26, or a variant thereof having at least about 80% sequence identity to the amino acid sequence of SEQ ID NO: 26, and a light chain variable domain comprising the amino acid sequence of SEQ ID NO: 43, or a variant thereof having at least about 80% sequence identity to the amino acid sequence of SEQ ID NO: 43. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 27, or a variant thereof having at least about 80% sequence identity to the amino acid sequence of SEQ ID NO: 27, and a light chain variable domain comprising the amino acid sequence of SEQ ID NO: 44, or a variant thereof having at least about 80% sequence identity to the amino acid sequence of SEQ ID NO: 44. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 28, or a variant thereof having at least about 80% sequence identity to the amino acid sequence of SEQ ID NO: 28, and a light chain variable domain comprising the amino acid sequence of SEQ ID NO: 44, or a variant thereof having at least about 80% sequence identity to the amino acid sequence of SEQ ID NO: 44. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 27, or a variant thereof having at least about 80% sequence identity to the amino acid sequence of SEQ ID NO: 27, and a light chain variable domain comprising the amino acid sequence of SEQ ID NO: 45, or a variant thereof having at least about 80% sequence identity to the amino acid sequence of SEQ ID NO: 45. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 28, or a variant thereof having at least about 80% sequence identity to the amino acid sequence of SEQ ID NO: 28, and a light chain variable domain comprising the amino acid sequence of SEQ ID NO: 45, or a variant thereof having at least about 80% sequence identity to the amino acid sequence of SEQ ID NO: 45. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 29, or a variant thereof having at least about 80% sequence identity to the amino acid sequence of SEQ ID NO: 29, and a light chain variable domain comprising the amino acid sequence of SEQ ID NO: 46, or a variant thereof having at least about 80% sequence identity to the amino acid sequence of SEQ ID NO: 46. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 30, or a variant thereof having at least about 80% sequence identity to the amino acid sequence of SEQ ID NO: 30, and a light chain variable domain comprising the amino acid sequence of SEQ ID NO: 47, or a variant thereof having at least about 80% sequence identity to the amino acid sequence of SEQ ID NO: 47. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 31, or a variant thereof having at least about 80% sequence identity to the amino acid sequence of SEQ ID NO: 31, and a light chain variable domain comprising the amino acid sequence of SEQ ID NO: 48, or a variant thereof having at least about 80% sequence identity to the amino acid sequence of SEQ ID NO: 48. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 31, or a variant thereof having at least about 80% sequence identity to the amino acid sequence of SEQ ID NO: 31, and a light chain variable domain comprising the amino acid sequence of SEQ ID NO: 49, or a variant thereof having at least about 80% sequence identity to the amino acid sequence of SEQ ID NO: 49. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 33, or a variant thereof having at least about 80% sequence identity to the amino acid sequence of SEQ ID NO: 33, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG1 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 53. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 53 and the light chain constant region comprises or consists of the amino acid sequence of SEQ ID NO: 55. In some embodiments, the heavy chain constant region comprises or consists of the amino acid sequence of SEQ ID NO: 53 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 19, or a variant thereof having at least about 80% sequence identity to the amino acid sequence of SEQ ID NO: 19, and a light chain variable domain comprising the amino acid sequence of SEQ ID NO: 38, or a variant thereof having at least about 80% sequence identity to the amino acid sequence of SEQ ID NO: 38. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 20, or a variant thereof having at least about 80% sequence identity to the amino acid sequence of SEQ ID NO: 20, and a light chain variable domain comprising the amino acid sequence of SEQ ID NO: 39, or a variant thereof having at least about 80% sequence identity to the amino acid sequence of SEQ ID NO: 39. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 40, or a variant thereof having at least about 80% sequence identity to the amino acid sequence of SEQ ID NO: 40. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 41, or a variant thereof having at least about 80% sequence identity to the amino acid sequence of SEQ ID NO: 41. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 21, or a variant thereof having at least about 80% sequence identity to the amino acid sequence of SEQ ID NO: 21, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 22, or a variant thereof having at least about 80% sequence identity to the amino acid sequence of SEQ ID NO: 22, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 23, or a variant thereof having at least about 80% sequence identity to the amino acid sequence of SEQ ID NO: 23, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 24, or a variant thereof having at least about 80% sequence identity to the amino acid sequence of SEQ ID NO: 24, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 25, or a variant thereof having at least about 80% sequence identity to the amino acid sequence of SEQ ID NO: 25, and a light chain variable domain comprising the amino acid sequence of SEQ ID NO: 42, or a variant thereof having at least about 80% sequence identity to the amino acid sequence of SEQ ID NO: 42. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 26, or a variant thereof having at least about 80% sequence identity to the amino acid sequence of SEQ ID NO: 26, and a light chain variable domain comprising the amino acid sequence of SEQ ID NO: 43, or a variant thereof having at least about 80% sequence identity to the amino acid sequence of SEQ ID NO: 43. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 27, or a variant thereof having at least about 80% sequence identity to the amino acid sequence of SEQ ID NO: 27, and a light chain variable domain comprising the amino acid sequence of SEQ ID NO: 44, or a variant thereof having at least about 80% sequence identity to the amino acid sequence of SEQ ID NO: 44. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 28, or a variant thereof having at least about 80% sequence identity to the amino acid sequence of SEQ ID NO: 28, and a light chain variable domain comprising the amino acid sequence of SEQ ID NO: 44, or a variant thereof having at least about 80% sequence identity to the amino acid sequence of SEQ ID NO: 44. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 27, or a variant thereof having at least about 80% sequence identity to the amino acid sequence of SEQ ID NO: 27, and a light chain variable domain comprising the amino acid sequence of SEQ ID NO: 45, or a variant thereof having at least about 80% sequence identity to the amino acid sequence of SEQ ID NO: 45. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 28, or a variant thereof having at least about 80% sequence identity to the amino acid sequence of SEQ ID NO: 28, and a light chain variable domain comprising the amino acid sequence of SEQ ID NO: 45, or a variant thereof having at least about 80% sequence identity to the amino acid sequence of SEQ ID NO: 45. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 29, or a variant thereof having at least about 80% sequence identity to the amino acid sequence of SEQ ID NO: 29, and a light chain variable domain comprising the amino acid sequence of SEQ ID NO: 46, or a variant thereof having at least about 80% sequence identity to the amino acid sequence of SEQ ID NO: 46. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 30, or a variant thereof having at least about 80% sequence identity to the amino acid sequence of SEQ ID NO: 30, and a light chain variable domain comprising the amino acid sequence of SEQ ID NO: 47, or a variant thereof having at least about 80% sequence identity to the amino acid sequence of SEQ ID NO: 47. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 31, or a variant thereof having at least about 80% sequence identity to the amino acid sequence of SEQ ID NO: 31, and a light chain variable domain comprising the amino acid sequence of SEQ ID NO: 48, or a variant thereof having at least about 80% sequence identity to the amino acid sequence of SEQ ID NO: 48. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 31, or a variant thereof having at least about 80% sequence identity to the amino acid sequence of SEQ ID NO: 31, and a light chain variable domain comprising the amino acid sequence of SEQ ID NO: 49, or a variant thereof having at least about 80% sequence identity to the amino acid sequence of SEQ ID NO: 49. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 50, or a variant thereof having at least about 80% sequence identity to the amino acid sequence of SEQ ID NO: 50. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 33, or a variant thereof having at least about 80% sequence identity to the amino acid sequence of SEQ ID NO: 33, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 51, or a variant thereof having at least about 80% sequence identity to the amino acid sequence of SEQ ID NO: 51. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 32, or a variant thereof having at least about 80% sequence identity to the amino acid sequence of SEQ ID NO: 32, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 34, or a variant thereof having at least about 80% sequence identity to the amino acid sequence of SEQ ID NO: 34, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 35, or a variant thereof having at least about 80% sequence identity to the amino acid sequence of SEQ ID NO: 35, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 36, or a variant thereof having at least about 80% sequence identity to the amino acid sequence of SEQ ID NO: 36, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

In some embodiments, there is provided a full-length anti-FasL antibody comprising IgG4 constant domains, wherein the anti-FasL antibody comprises a heavy chain variable domain comprising the amino acid sequence of SEQ ID NO: 37, or a variant thereof having at least about 80% sequence identity to the amino acid sequence of SEQ ID NO: 37, and a light chain variable domain comprising the amino acid sequence of SEQ ID NO: 52, or a variant thereof having at least about 80% sequence identity to the amino acid sequence of SEQ ID NO: 52. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56. In some embodiments, the heavy chain constant region comprises or consists of the amino acid sequence of SEQ ID NO: 54 and the light chain constant region comprises or consists of the amino acid sequence of SEQ ID NO: 55. In some embodiments, the heavy chain constant region comprises or consists of the amino acid sequence of SEQ ID NO: 54 and the light chain constant region comprises or consists of the amino acid sequence of SEQ ID NO: 56.

### Binding Affinity

Binding affinity can be indicated by Kd, Koff, Kon, or Ka. The term “Koff”, as used herein, is intended to refer to the off-rate constant for dissociation of an antibody from the antibody/antigen complex, as determined from a kinetic selection set up. The term “Kon”, as used herein, is intended to refer to the on-rate constant for association of an antibody to the antigen to form the antibody/antigen complex. The term dissociation constant “Kd”, as used herein, refers to the dissociation constant of a particular antibody-antigen interaction, and describes the concentration of antigen required to occupy one half of all of the antibody-binding domains present in a solution of antibody molecules at equilibrium, and is equal to Koff/Kon. The measurement of Kd presupposes that all binding agents are in solution. In the case where the antibody is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antibody moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M. An antibody that specifically binds to a target may have a Kd of, for example, ≤10⁻⁷ M, ≤10⁻⁸ M, ≤10⁻⁹ M, ≤10⁻¹⁰ M, ≤10⁻¹¹ M, ≤10⁻¹², or ≤10⁻¹³ M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

In some embodiments, the anti-FasL antibody specifically binds to a target FasL with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M). Thus in some embodiments, the Kd of the binding between the anti-FasL antibody and FasL, is about 10⁻⁷ M to about 10⁻¹³ M, about 1×10⁻⁷ M to about 5×10⁻¹³ M, about 10⁻⁷ M to about 10⁻¹² M, about 10⁻⁷ M to about 10⁻¹¹ M, about 10⁻⁷ M to about 10⁻¹⁰ M, about 10⁻⁷ M to about 10⁻⁹ M, about 10⁻⁸ M to about 10⁻¹¹ M, about 1×10⁻⁸ M to about 5×10⁻¹³ M, about 10⁻⁸ M to about 10⁻¹² M, about 10⁻⁸ M to about 10⁻¹¹ M, about 10⁻⁸ M to about 10⁻¹⁰ M, about 10⁻⁸ M to about 10⁻⁹ M, about 5×10⁻⁹ M to about 1×10⁻¹³ M, about 5×10⁻⁹ M to about 1×10⁻¹² M, about 5×10⁻⁹ M to about 1×10⁻¹¹ M, about 5×10⁻⁹ M to about 1×10⁻¹⁰ M, about 10⁻⁹ M to about 10⁻¹³ M, about 10⁻⁹ M to about 10⁻¹² M, about 10⁻⁹ M to about 10⁻¹¹ M, about 10⁻⁹ M to about 10⁻¹⁰ M, about 5×10⁻¹⁰ M to about 1×10⁻¹³ M, about 5×10⁻¹⁰ M to about 1×10⁻¹² M, about 5×10⁻¹⁰ M to about 1×10⁻¹¹ M, about 10⁻¹⁰ M to about 10⁻¹³ M, about 1×10⁻¹⁰ M to about 5×10⁻¹³ M, about 1×10⁻¹⁰ M to about 1×10⁻¹² M, about 1×10⁻¹⁰ M to about 5×10⁻¹² M, about 1×10⁻¹⁰ M to about 1×10⁻¹¹ M, about 10⁻¹¹ M to about 10⁻¹³ M, about 1×10⁻¹¹ M to about 5×10⁻¹³ M, about 10⁻¹¹ M to about 10⁻¹² M, or about 10⁻¹² M to about 10⁻¹³ M. In some embodiments, the Kd of the binding between the anti-FasL antibody and a FasL is about 10⁻⁷ M to about 10⁻¹³ M.

In some embodiments, the Kd of the binding between the anti-FasL antibody and a non-target is more than the Kd of the binding between the anti-FasL antibody and the target, and is herein referred to in some embodiments as the binding affinity, of the anti-FasL antibody to the target (e.g., FasL) is higher than that to a non-target. In some embodiments, the non-target is an antigen that is not FasL. In some embodiments, the Kd of the binding between the anti-FasL antibody (against FasL) and a non-FasL target can be at least about 10 times, such as about 10-100 times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times of the Kd of the binding between the anti-FasL antibody and a target FasL.

In some embodiments, the anti-FasL antibody binds to a non-target with a Kd of about 10⁻¹ M to about 10⁻⁶ M (such as about 10⁻¹ M to about 10⁻⁶ M, about 10⁻¹ M to about 10⁻⁵ M, or about 10⁻². M to about 10⁻⁴ M). In some embodiments, the non-target is an antigen that is not FasL. Thus in some embodiments, the Kd of the binding between the anti-FasL antibody and a non-FasL target is about 10⁻¹ M to about 10⁻⁶ M, about 1×10⁻¹ M to about 5×10⁻⁶ M, about 10⁻¹ M to about 10⁻⁵ M, about 1×10⁻¹ M to about 5×10⁻⁵ M, about 10⁻¹ M to about 10⁻⁴ M, about 1×10⁻¹ M to about 5×10⁻⁴ M, about 10⁻¹ M to about 10⁻³ M, about 1×10⁻¹ M to about 5×10⁻³ M, about 10⁻¹ M to about 10⁻² M, about 10⁻² M to about 10⁻⁶ M, about 1×10⁻² M to about 5×10⁻⁶ M, about 10⁻² M to about 10⁻⁵ M, about 1×10⁻² M to about 5×10⁻⁵ M, about 10⁻² M to about 10⁻⁴ M, about 1×10⁻² M to about 5×10⁻⁴ M, about 10⁻² M to about 10⁻³ M, about 10⁻³ M to about 10⁻³ M, about 1×10⁻³ M to about 5×10⁻⁶ M, about 10⁻³ M to about 10⁻⁵ M, about 1×10⁻³ M to about 5×10⁻⁶ M, about 10⁻³ M to about 10⁻⁴ M, about 10⁻⁴ M to about 10⁻⁶ M, about 1×10⁻⁴ M to about 5×10⁻⁶ M, about 10⁻⁴ M to about 10⁻⁵ M, or about 10⁻⁵ M to about 10⁻⁶ M.

In some embodiments, when referring to that the anti-FasL antibody specifically recognizes a target FasL at a high binding affinity, and binds to a non-target at a low binding affinity, the anti-FasL antibody will bind to the target FasL with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M), and will bind to the non-target with a Kd of about 10⁻¹ M to about 10⁻⁶ M (such as about 10⁻¹ M to about 10⁻⁶ M, about 10⁻¹ M to about 10⁻⁵ M, or about 10⁻³ M to about 10⁻⁴ M).

In some embodiments, when referring to that the anti-FasL antibody specifically recognizes FasL, the binding affinity of the anti-FasL antibody is compared to that of a control anti-FasL antibody (such as 119-4A or APG101 or MAB126-100). In some embodiments, the Kd of the binding between the control anti-FasL antibody and FasL can be at least about 2 times, such as about 2 times, about 3 times, about 4 times, about 5 times, about 6 times, about 7 times, about 8 times, about 9 times, about 10 times, about 10-100 times, about 100-1000 times, about 103-104 times of the Kd of the binding between the anti-FasL antibody described herein and FasL.

### Nucleic Acid

Nucleic acid molecules encoding the anti-FasL antibodies are also contemplated. In some embodiments, there is provided a nucleic acid (or a set of nucleic acids) encoding a full-length anti-FasL antibody, including any of the full-length anti-FasL antibodies described herein. In some embodiments, the nucleic acid (or a set of nucleic acids) encoding the anti-FasL antibody described herein may further comprises a nucleic acid sequence encoding a peptide tag (such as protein purification tag, e.g., His-tag, HA tag).

Also contemplated here are isolated host cells comprising an anti-FasL antibody, an isolated nucleic acid encoding the polypeptide components of the anti-FasL antibody, or a vector comprising a nucleic acid encoding the polypeptide components of the anti-FasL antibody described herein.

The present application also includes variants to these nucleic acid sequences. For example, the variants include nucleotide sequences that hybridize to the nucleic acid sequences encoding the anti-FasL antibodies of the present application under at least moderately stringent hybridization conditions.

The present application also provides vectors in which a nucleic acid of the present application is inserted.

In brief summary, the expression of an anti-FasL antibody (e.g., full-length anti-FasL antibody) by a natural or synthetic nucleic acid encoding the anti-FasL antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequences.

The nucleic acids of the present application may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV) human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-FasL antibody is inducible. In some embodiments, a nucleic acid sequence encoding the anti-FasL antibody is operably linked to an inducible promoter, including any inducible promoter described herein.

### Inducible Promoters

The use of an inducible promoter provides a molecular switch capable of turning on expression of the polynucleotide sequence to which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad Sci. USA 90:5603-5607), synthetic ligand-regulated elements (see, e.g., Spencer, D. M. et al. (1993) Science 262: 1019-1024) and ionizing radiation-regulated elements (e.g., see Manome, Y. et al. (1993) Biochemistry 32: 10607-10613; Datta, R. et al. (1992) Proc. Natl. Acad Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the anti-FasL antibody is the Tet system. In some embodiments, the inducible promoter system for use to express the anti-FasL antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, β-galactosidase, chloramphenicol acetyl transferase, secreted alkaline phosphatase, or the green fluorescent protein gene (e.g., Ui-Tel et al, 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-FasL antibody according to any of the full-length anti-FasL antibodies described herein. In some embodiments, the nucleic acid comprises one or more nucleic acid sequences encoding the heavy and light chains of the full-length anti-FasL antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

### Preparation of Anti-FasL Antibodies

In some embodiments, the anti-FasL antibody is a monoclonal antibody or derived from a monoclonal antibody. In some embodiments, the anti-FasL antibody comprises V_{H} and V_{L} domains, or variants thereof, from the monoclonal antibody. In some embodiments, the anti-FasL antibody further comprises C_{H}1 and C_{L} domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal Biochem., 107:220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-FasL antibodies described herein, the anti-FasL antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad Sci. USA 101(34): 12467-12472 (2004); and Lee et al., J. Immunol. Methods 284(1-2): 119-132(2004).

In certain phage display methods, repertoires of VA and V_{L} genes are separately cloned by polymerase chain reaction (PCR) and recombined randomly in phage libraries, which can then be screened for antigen-binding phage as described in Winter et al, Ann. Rev. Immunol, 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-FasL antibodies can be prepared using phage display to screen libraries for anti-FasL antibody moieties specific to the target FasL. The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naive human library constructed from DNA extracted from human PMBCs and spleens from healthy donors, encompassing all human heavy and light chain subfamilies. In some embodiments, the library is a naive human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target FasL with high affinity can be selected by iterative binding of phage to the target FasL, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL 1-Blue, for expression and purification. The panning can be performed for multiple (such as about any of 2, 3, 4, 5, 6 or more) rounds with solution panning, cell panning, or a combination of both, to enrich for phage clones binding specifically to the target FasL. Enriched phage clones can be tested for specific binding to the target FasL by any methods known in the art, including for example ELISA and FACS.

An alternative method for screening antibody libraries is to display the protein on the surface of yeast cells. Wittrup et al. (U.S. Pat. Nos. 6,699,658 and 6,696,251) have developed a method for a yeast cell display library. In this yeast display system, a component involves the yeast agglutinin protein (Aga1), which is anchored to the yeast cell wall. Another component involves a second subunit of the agglutinin protein Aga2, which can display on the surface yeast cells through disulfide bonds to Aga1 protein. The protein Aga1 is expressed from a yeast chromosome after the Aga1 gene integration. A library of single chain variable fragments (scFv) is fused genetically to Aga2 sequence in the yeast display plasmid, which, after transformation, is maintained in yeast episomally with a nutritional marker. Both of the Aga1 and Aga2 proteins were expressed under the control of the galactose-inducible promoter.

Human antibody V gene repertoire (V_{H} and V_{K} fragments) are obtained by PCR method using a pool of degenerate primers (Sblattero, D. & Bradbury, A. Immunotechnology 3, 271-278 1998). The PCR templates are from the commercially available RNAs or cDNAs, including PBMC, spleen, lymph nodes, bone marrow and tonsils. Separate V_{H} and V_{K} PCR libraries were combined, then assembled together in the scFv format by overlap extension PCR (Sheets, M. D. et al., Proc. Natl. Acad Sci. USA 95, 6157-6162 1998.). To construct the yeast scFv display library, the resultant scFv PCR products are cloned into the yeast display plasmid in the yeasts by homologous recombination. (Chao, G, et al, Nat Protoc. 2006; 1(2):755-68. Miller K D, et al, Current Protocols in Cytometry 4.7.1-4.7.30, 2008).

The anti-FasL antibodies can be discovered using mammalian cell display systems in which antibody moieties are displayed on the cell surface and those specific to the target FasL are isolated by the antigen-guided screening method, as described in U.S. Pat. No. 7,732,195B2. A Chinese hamster ovary (CHO) cell library representing a large set of human IgG antibody genes can be established and used to discover the clones expressing high-affinity antibody genes. Another display system has been developed to enable simultaneous high-level cell surface display and secretion of the same protein through alternate splicing, where the displayed protein phenotype remains linked to genotype, allowing soluble secreted antibody to be simultaneously characterized in biophysical and cell-based functional assays. This approach overcomes many limitations of previous mammalian cell display, enabling direct selection and maturation of antibodies in the form of full-length, glycosylated IgGs (Peter M. Bowers, et al., Methods 2014, 65:44-56). Transient expression systems are suitable for a single round of antigen selection before recovery of the antibody genes and therefore most useful for the selection of antibodies from smaller libraries. Stable episomal vectors offer an attractive alternative. Episomal vectors can be transfected at high efficiency and stably maintained at low copy number, permitting multiple rounds of panning and the resolution of more complex antibody libraries.

The IgG library is based on germline sequence V-gene segments joined to rearranged (D)J regions isolated from a panel of human donors. RNA collected from 2000 human blood samples was reverse-transcribed into cDNA, and the V_{H} and V_{K} fragments were amplified using V_{H}- and V_{K}-specific primers and purified by gel extraction. IgG libraries were generated by sub-cloning the V_{H} and V_{K} fragments into the display vectors containing IgG1 or K constant regions respectively and then electroporating into or transducing 293T cells. To generate the scFv antibody display library, scFvs were generated by linking V_{H} and V_{K}, and then sub-cloned into the display vector, which were then electroporated into or transduce 293T cells. As we known, the IgG library is based on germline sequence V-gene segments joined to rearranged (D)J regions isolated from a panel of donors, the donor can be a mouse, rat, rabbit, or monkey.

Monoclonal antibodies can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or FasL-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

### Human and Humanized Antibodies

The anti-FasL antibodies (e.g., full-length anti-FasL antibodies) can be humanized antibodies or human antibodies. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al, Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al, Bio/Technology, 10: 779-783 (1992); Lonberg et al, Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol, 222:581 (1991). The techniques of Cole et al. and Boerner et al. are also available for the preparation of human monoclonal antibodies. Cole et al, Monoclonal Antibodies and Cancer Therapy, Alan R. Liss, p. 77 (1985) and Boemer et al., J. Immunol., 147(1): 86-95 (1991).

### Anti-FasL Antibody Variants

In some embodiments, amino acid sequences of the anti-FasL antibody variants (e.g., full-length anti-FasL antibody) provided herein are contemplated. For example, it may be desirable to improve the binding affinity and/or other biological properties of the antibody. Amino acid sequences of an antibody variant may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, anti-FasL antibody variants having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding, decreased immunogenicity, or improved ADCC or CDC.

Conservative substitutions are shown in Table 4 below.

TABLE 4
CONSERVATIVE SUBSTITUTIONS

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on the activity of inhibiting cell apoptosis or improving antibody affinity). Alterations (e.g., substitutions) may be made in HVRs, e.g., to improve bioactivity based on antibody affinity. Such alterations may be made in HVR “hotspots”, e.g., residues encoded by codons that undergo mutation at high frequency during the somatic maturation process (see, e.g., Chowdhury, Methods Mol. Biol 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al., in Methods in Molecular Biology 178:1-37 (O'Brien et al, ed, Human Press, Totowa NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Fc Region Variants

In some embodiments, one or more amino acid modifications may be introduced into the Fc region of an antibody (e.g., a full-length anti-FasL antibody or anti-FasL Fc fusion protein) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al., J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-FasL antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the application contemplates an anti-FasL antibody variant (such as a full-length anti-FasL antibody variant) comprising an Fc region that possesses some but not all effector functions, which makes it a desirable candidate for applications in which the half-life of the anti-FasL antibody in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al., Proc. Nat'l Acad Sci. USA 83:7059-7063 (1986)) and Hellstrom, I. et al., Proc. Nat'l Acad Sci. USA 82:1499-1502 (1985); U.S. Pat. No. 5,821,337 (see Bruggemann, M. et al, J. Exp. Med 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol Methods 202:163 (1996); Cragg, M. S. et al, Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al, J. Biol Chem. 9(2): 6591-6604 (2001).) In some embodiments, there is provided an anti-FasL antibody (such as a full-length anti-FasL antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-FasL antibody (e.g., full-length anti-FasL antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-FasL antibody (such as a full-length anti-FasL antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-FasL antibodies (such as full-length anti-FasL antibodies) comprising any of the Fc variants described herein, or combinations thereof, are contemplated.

### Glycosylation Variants

In some embodiments, an anti-FasL antibody (such as a full-length anti-FasL antibody) provided herein is altered to increase or decrease the extent to which the anti-FasL antibody is glycosylated. Addition or deletion of glycosylation sites to an anti-FasL antibody may be conveniently accomplished by altering the amino acid sequence of the anti-FasL antibody or polypeptide portion thereof such that one or more glycosylation sites are created or removed.

Wherein the anti-FasL antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al, TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-FasL antibody of the application may be made in order to create anti-FasL antibody variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al, J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-FasL antibody (such as a full-length anti-FasL antibody) variants are provided comprising an Fc region wherein a carbohydrate structure attached to the Fc region has reduced fucose or lacks fucose, which may improve ADCC function. Specifically, anti-FasL antibodies are contemplated herein that have reduced fucose relative to the amount of fucose on the same anti-FasL antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-FasL antibody is one wherein less than about 50%, 40%, 30%, 20%, 10%, or 5% of the N-linked glycans thereon comprise fucose. For example, the amount of fucose in such an anti-FasL antibody may be from 1% to 80%, from 1% to 65%, from 5% to 65% or from 20% to 40%. In some embodiments, the anti-FasL antibody is one wherein none of the N-linked glycans thereon comprise fucose, i.e., wherein the anti-FasL antibody is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al, J. Mol. Biol 336:1239-1249 (2004); Yamane-Ohnuki et al., Biotech Bioeng. 87: 614 (2004). Examples of cell lines capable of producing defucosylated antibodies include Lec13 CHO cells deficient in protein fucosylation (Ripka et al., Arch Biochem. Biophys 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng. 94(4):680-688 (2006); and WO2003/085107).

Anti-FasL antibody (such as a full-length anti-FasL antibody) variants are further provided with bisected oligosaccharides, e.g., in which a biantennary oligosaccharide attached to the Fc region of the anti-FasL antibody is bisected by GlcNAc. Such anti-FasL antibody (such as a full-length anti-FasL antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al), and Ferrara et al., Biotechnology and Bioengineering, 93(5): 851-861 (2006). Anti-FasL antibody (such as full-length anti-FasL antibody) variants with at least one galactose residue in the oligosaccharide attached to the Fc region are also provided. Such anti-FasL antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-FasL antibody (such as a full-length anti-FasL antibody) variants comprising an Fc region are capable of binding to an FcγRIII. In some embodiments, the anti-FasL antibody (such as a full-length anti-FasL antibody) variants comprising an Fc region have ADCC activity in the presence of human effector cells (e.g., T cell) or have increased ADCC activity in the presence of human effector cells compared to the otherwise same anti-FasL antibody (such as a full-length anti-FasL antibody) comprising a human wild-type IgG1Fc region.

### Cysteine Engineered Variants

In some embodiments, it may be desirable to create cysteine engineered anti-FasL antibodies (such as a full-length anti-FasL antibody) in which one or more amino acid residues are substituted with cysteine residues. In some embodiments, the substituted residues occur at accessible sites of the anti-FasL antibody. By substituting those residues with cysteine, reactive thiol groups are thereby positioned at accessible sites of the anti-FasL antibody and may be used to conjugate the anti-FasL antibody to other moieties, such as drug moieties or linker-drug moieties, to create an anti-FasL immunoconjugate, as described further herein. Cysteine engineered anti-FasL antibodies (e.g., full-length anti-FasL antibodies) may be generated as described, e.g., in U.S. Pat. No. 7,521,541.

### Derivatives

In some embodiments, an anti-FasL antibody (such as a full-length anti-FasL antibody) provided herein may be further modified to contain additional non-proteinaceous moieties that are known in the art and readily available. The moieties suitable for derivatization of the anti-FasL antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-FasL antibody may vary, and if more than one polymer are attached, they can be the same or different molecules. In general, the number and/or type of polymers used for derivatization can be determined based on considerations including, but not limited to, the particular properties or functions of anti-FasL antibody to be improved, whether the anti-FasL antibody derivative will be used in a therapy under defined conditions, etc.

### Pharmaceutical Compositions

Also provided herein are compositions (such as pharmaceutical compositions, also referred to herein as formulations) comprising any of the anti-FasL antibodies (such as a full-length anti-FasL antibody), nucleic acids encoding the antibodies, vectors comprising the nucleic acids encoding the antibodies, or host cells comprising the nucleic acids or vectors described herein. In some embodiments, there is provided a pharmaceutical composition comprising any one of the anti-FasL antibodies described herein and a pharmaceutically acceptable carrier.

Suitable formulations of the anti-FasL antibodies are obtained by mixing an anti-FasL antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-FasL antibodies of this application into cells.

The formulation herein may also contain one or more active compounds in addition to the anti-FasL antibody (such as a full-length anti-FasL antibody) as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-FasL antibody. Such molecules are suitably present in combination in amounts that are effective for the purpose intended. The effective amount of such other agents depends on the amount of anti-FasL antibody present in the formulation, the type of disease or disorder or treatment, and other factors discussed above. These are generally used in the same dosages and with administration routes as described herein or about from 1 to 99% of the heretofore employed dosages.

The anti-FasL antibodies (e.g., full-length anti-FasL antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-FasL antibodies (e.g., full-length anti-FasL antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D(−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-FasL antibodies depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-FasL antibody (such as a full-length anti-FasL antibody) is formulated in a buffer comprising a citrate, NaCl, acetate, succinate, glycine, polysorbate 80 (Tween 80), or any combination of the foregoing.

The formulations to be used for in vivo administration must be sterile. This is readily accomplished by, e.g., filtration through sterile filtration membranes.

### Methods of Treatment Using Anti-FasL Antibodies

The anti-FasL antibodies (e.g., full-length anti-FasL antibodies) and/or compositions of the application can be administered to individuals (e.g., mammals such as humans) to treat a disease and/or condition associated with disorder of FasL-Fas signaling pathway (e.g., inflammatory diseases, autoimmune diseases or cancer). These diseases include, but are not limited to, pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer. In particular, FASL-positive cancer diseases are characterized by the expression of FasL on the cell surface. In some embodiments, cancer can be recognized as FASL-positive if at least 1%, at least 2%, at least 5%, at least 10%, at least 20%, or at least 50% of cells in a cancer sample express FasL. In some embodiments, the number of FASL-positive cells can be detected by counting cells in a microscopic section. The present application thus in some embodiments provides a method of treating a disease and/or condition associated with disorder of FasL-Fas signaling pathway (e.g., inflammatory diseases, autoimmune diseases or cancer) comprising administering to the individual an effective amount of a composition (such as a pharmaceutical composition) comprising an anti-FasL antibody (e.g., a full-length anti-FasL antibody), such as any one of the anti-FasL antibodies (e.g., full-length anti-FasL antibodies) described herein. In some embodiments, the individual is human.

For example, in some embodiments, there is provided a method of treating an individual having a disease associated with FasL-Fas signaling pathway (e.g., inflammatory diseases, autoimmune diseases or cancer) comprising administering to the individual an effective amount of a pharmaceutical composition comprising an anti-FasL antibody (e.g., full-length anti-FasL antibody) specifically binding to an epitope on human FasL, wherein the epitope comprises amino acid residues of human FasL. In some embodiments, the anti-FasL antibody is a full-length antibody. In some embodiments, the full-length anti-FasL antibody is an IgG1 or IgG4 antibody. In some embodiments, the disease or condition is selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer. In particular, FASL-positive cancer diseases are characterized by the expression of FasL on the cell surface. In some embodiments, cancer can be recognized as FASL-positive if at least 1%, at least 2%, at least 5%, at least 10%, at least 20%, or at least 50% of cells in a cancer sample express FasL. In some embodiments, the number of FASL-positive cells can be detected by counting cells in a microscopic section. In some embodiments, the individual is human.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 7, or a variant thereof comprising up to 5 amino acid substitutions in the HC-CDRs; and a V_{L} comprising an LC-CDR1 comprising the amino acid sequence of SEQ ID NO: 10, an LC-CDR2 comprising the amino acid sequence of SEQ ID NO: 13, and an LC-CDR3 comprising the amino acid sequence of SEQ ID NO: 16, or a variant thereof comprising up to 5 amino acid substitutions in the LC-CDRs. In some embodiments, the anti-FasL antibody is a full-length antibody. In some embodiments, the full-length anti-FasL antibody is an IgG1 or IgG4 antibody. In some embodiments, the disease or condition is selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer. In some embodiments, the individual is human.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising the amino acid sequence of any one of SEQ ID NOs: 19-25 or a variant thereof having at least about 80% sequence identity to the amino acid sequence of any one of SEQ ID NOs: 19-25, and a V_{L} comprising the amino acid sequence of any one of SEQ ID NOs: 38-42, or a variant thereof having at least about 80% sequence identity to the amino acid sequence of any one of SEQ ID NOs: 38-42.

In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 19 or a variant thereof having at least about 80% sequence identity to the amino acid sequence of SEQ ID NO: 19, and a V_{L} comprising the amino acid sequence of SEQ ID NO: 38 or a variant thereof having at least about 80% sequence identity to the amino acid sequence of SEQ ID NO: 38. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 20 or a variant thereof having at least about 80% sequence identity to the amino acid sequence of SEQ ID NO: 20, and a V_{L} comprising the amino acid sequence of SEQ ID NO: 39 or a variant thereof having at least about 80% sequence identity to the amino acid sequence of SEQ ID NO: 39. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 21 or a variant thereof having at least about 80% sequence identity to the amino acid sequence of SEQ ID NO: 21, and a V_{L} comprising the amino acid sequence of SEQ ID NO: 40 or a variant thereof having at least about 80% sequence identity to the amino acid sequence of SEQ ID NO: 40. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 22 or a variant thereof having at least about 80% sequence identity to the amino acid sequence of SEQ ID NO: 22, and a V_{L} comprising the amino acid sequence of SEQ ID NO: 40 or a variant thereof having at least about 80% sequence identity to the amino acid sequence of SEQ ID NO: 40. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 23 or a variant thereof having at least about 80% sequence identity to the amino acid sequence of SEQ ID NO: 23, and a V_{L} comprising the amino acid sequence of SEQ ID NO: 40 or a variant thereof having at least about 80% sequence identity to the amino acid sequence of SEQ ID NO: 40. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 24 or a variant thereof having at least about 80% sequence identity to the amino acid sequence of SEQ ID NO: 24, and a V_{L} comprising the amino acid sequence of SEQ ID NO: 40 or a variant thereof having at least about 80% sequence identity to the amino acid sequence of SEQ ID NO: 40. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 25 or a variant thereof having at least about 80% sequence identity to the amino acid sequence of SEQ ID NO: 25, and a V_{L} comprising the amino acid sequence of SEQ ID NO: 40 or a variant thereof having at least about 80% sequence identity to the amino acid sequence of SEQ ID NO: 40. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 21 or a variant thereof having at least about 80% sequence identity to the amino acid sequence of SEQ ID NO: 21, and a V_{L} comprising the amino acid sequence of SEQ ID NO: 41 or a variant thereof having at least about 80% sequence identity to the amino acid sequence of SEQ ID NO: 41. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 22 or a variant thereof having at least about 80% sequence identity to the amino acid sequence of SEQ ID NO: 22, and a V_{L} comprising the amino acid sequence of SEQ ID NO: 41 or a variant thereof having at least about 80% sequence identity to the amino acid sequence of SEQ ID NO: 41. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 23 or a variant thereof having at least about 80% sequence identity to the amino acid sequence of SEQ ID NO: 23, and a V_{L} comprising the amino acid sequence of SEQ ID NO: 41 or a variant thereof having at least about 80% sequence identity to the amino acid sequence of SEQ ID NO: 41. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 24 or a variant thereof having at least about 80% sequence identity to the amino acid sequence of SEQ ID NO: 24, and a V_{L} comprising the amino acid sequence of SEQ ID NO: 41 or a variant thereof having at least about 80% sequence identity to the amino acid sequence of SEQ ID NO: 41. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 25 or a variant thereof having at least about 80% sequence identity to the amino acid sequence of SEQ ID NO: 25, and a V_{L} comprising the amino acid sequence of SEQ ID NO: 41 or a variant thereof having at least about 80% sequence identity to the amino acid sequence of SEQ ID NO: 41. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 21 or a variant thereof having at least about 80% sequence identity to the amino acid sequence of SEQ ID NO: 21, and a V_{L} comprising the amino acid sequence of SEQ ID NO: 42 or a variant thereof having at least about 80% sequence identity to the amino acid sequence of SEQ ID NO: 42. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 22 or a variant thereof having at least about 80% sequence identity to the amino acid sequence of SEQ ID NO: 22, and a V_{L} comprising the amino acid sequence of SEQ ID NO: 42 or a variant thereof having at least about 80% sequence identity to the amino acid sequence of SEQ ID NO: 42. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 23 or a variant thereof having at least about 80% sequence identity to the amino acid sequence of SEQ ID NO: 23, and a V_{L} comprising the amino acid sequence of SEQ ID NO: 42 or a variant thereof having at least about 80% sequence identity to the amino acid sequence of SEQ ID NO: 42. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 24 or a variant thereof having at least about 80% sequence identity to the amino acid sequence of SEQ ID NO: 24, and a V_{L} comprising the amino acid sequence of SEQ ID NO: 42 or a variant thereof having at least about 80% sequence identity to the amino acid sequence of SEQ ID NO: 42. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 25 or a variant thereof having at least about 80% sequence identity to the amino acid sequence of SEQ ID NO: 25, and a V_{L} comprising the amino acid sequence of SEQ ID NO: 42 or a variant thereof having at least about 80% sequence identity to the amino acid sequence of SEQ ID NO: 42. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising an HC-CDR1 comprising the amino acid sequence of SEQ ID NO: 2, an HC-CDR2 comprising the amino acid sequence of SEQ ID NO: 5, and an HC-CDR3 comprising the amino acid sequence of SEQ ID NO: 8, or a variant thereof comprising up to 5 amino acid substitutions in the HC-CDRs; and a V_{L} comprising an LC-CDR1 comprising the amino acid sequence of SEQ ID NO: 11, an LC-CDR2 comprising the amino acid sequence of SEQ ID NO: 14, and an LC-CDR3 comprising the amino acid sequence of SEQ ID NO: 17, or a variant thereof comprising up to 5 amino acid substitutions in the LC-CDRs. In some embodiments, the anti-FasL antibody is a full-length antibody. In some embodiments, the full-length anti-FasL antibody is an IgG1 or IgG4 antibody. In some embodiments, the disease or condition is selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer. In some embodiments, the individual is human.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising the amino acid sequence of any one of SEQ ID NOs: 26-28 or a variant thereof having at least about 80% sequence identity to the amino acid sequence of any one of SEQ ID NOs: 26-28, and a V_{L} comprising the amino acid sequence of any one of SEQ ID NOs: 43-45, or a variant thereof having at least about 80% sequence identity to the amino acid sequence of any one of SEQ ID NOs: 43-45.

In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 26 or a variant thereof having at least about 80% sequence identity to the amino acid sequence of SEQ ID NO: 26, and a V_{L} comprising the amino acid sequence of SEQ ID NO: 43 or a variant thereof having at least about 80% sequence identity to the amino acid sequence of SEQ ID NO: 43. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 27 or a variant thereof having at least about 80% sequence identity to the amino acid sequence of SEQ ID NO: 27, and a V_{L} comprising the amino acid sequence of SEQ ID NO: 44 or a variant thereof having at least about 80% sequence identity to the amino acid sequence of SEQ ID NO: 44. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 28 or a variant thereof having at least about 80% sequence identity to the amino acid sequence of SEQ ID NO: 28, and a V_{L} comprising the amino acid sequence of SEQ ID NO: 44 or a variant thereof having at least about 80% sequence identity to the amino acid sequence of SEQ ID NO: 44. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 27 or a variant thereof having at least about 80% sequence identity to the amino acid sequence of SEQ ID NO: 27, and a V_{L} comprising the amino acid sequence of SEQ ID NO: 45 or a variant thereof having at least about 80% sequence identity to the amino acid sequence of SEQ ID NO: 45. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 28 or a variant thereof having at least about 80% sequence identity to the amino acid sequence of SEQ ID NO: 28, and a V_{L} comprising the amino acid sequence of SEQ ID NO: 45 or a variant thereof having at least about 80% sequence identity to the amino acid sequence of SEQ ID NO: 45. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising an HC-CDR1 comprising the amino acid sequence of SEQ ID NO: 3, an HC-CDR2 comprising the amino acid sequence of SEQ ID NO: 6, and an HC-CDR3 comprising the amino acid sequence of SEQ ID NO: 9, or a variant thereof comprising up to 5 amino acid substitutions in the HC-CDRs; and a V_{L} comprising an LC-CDR1 comprising the amino acid sequence of SEQ ID NO: 12, an LC-CDR2 comprising the amino acid sequence of SEQ ID NO: 15, and an LC-CDR3 comprising the amino acid sequence of SEQ ID NO: 18, or a variant thereof comprising up to 5 amino acid substitutions in the LC-CDRs. In some embodiments, the anti-FasL antibody is a full-length antibody. In some embodiments, the full-length anti-FasL antibody is an IgG1 or IgG4 antibody. In some embodiments, the disease or condition is selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer. In some embodiments, the cancer is a FasL-positive cancer. In some embodiments, the individual is human.

In some embodiments, there is provided a method of treating an individual having a disease associated with associated with FasL-Fas signaling pathway (e.g., inflammatory diseases, autoimmune diseases or cancer) comprising administering to the individual an effective amount of a pharmaceutical composition comprising an anti-FasL antibody comprising: a V_{H} comprising the amino acid sequence of any one of SEQ ID NOs: 29-37 or a variant thereof having at least about 80% sequence identity to the amino acid sequence of any one of SEQ ID NOs: 29-37, and a V_{L} comprising the amino acid sequence of any one of SEQ ID NOs: 46-52, or a variant thereof having at least about 80% sequence identity to the amino acid sequence of any one of SEQ ID NOs: 46-52.

In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 29 or a variant thereof having at least about 80% sequence identity to the amino acid sequence of SEQ ID NO: 29, and a V_{L} comprising the amino acid sequence of SEQ ID NO: 46 or a variant thereof having at least about 80% sequence identity to the amino acid sequence of SEQ ID NO: 46. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 30 or a variant thereof having at least about 80% sequence identity to the amino acid sequence of SEQ ID NO: 30, and a V_{L} comprising the amino acid sequence of SEQ ID NO: 47 or a variant thereof having at least about 80% sequence identity to the amino acid sequence of SEQ ID NO: 47. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 31 or a variant thereof having at least about 80% sequence identity to the amino acid sequence of SEQ ID NO: 31, and a V_{L} comprising the amino acid sequence of SEQ ID NO: 48 or a variant thereof having at least about 80% sequence identity to the amino acid sequence of SEQ ID NO: 48. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 31 or a variant thereof having at least about 80% sequence identity to the amino acid sequence of SEQ ID NO: 31, and a V_{L} comprising the amino acid sequence of SEQ ID NO: 49 or a variant thereof having at least about 80% sequence identity to the amino acid sequence of SEQ ID NO: 49. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 32 or a variant thereof having at least about 80% sequence identity to the amino acid sequence of SEQ ID NO: 32, and a V_{L} comprising the amino acid sequence of SEQ ID NO: 50 or a variant thereof having at least about 80% sequence identity to the amino acid sequence of SEQ ID NO: 50. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 34 or a variant thereof having at least about 80% sequence identity to the amino acid sequence of SEQ ID NO: 34, and a V_{L} comprising the amino acid sequence of SEQ ID NO: 50 or a variant thereof having at least about 80% sequence identity to the amino acid sequence of SEQ ID NO: 50. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 35 or a variant thereof having at least about 80% sequence identity to the amino acid sequence of SEQ ID NO: 35, and a V_{L} comprising the amino acid sequence of SEQ ID NO: 50 or a variant thereof having at least about 80% sequence identity to the amino acid sequence of SEQ ID NO: 50. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 36 or a variant thereof having at least about 80% sequence identity to the amino acid sequence of SEQ ID NO: 36, and a V_{L} comprising the amino acid sequence of SEQ ID NO: 50 or a variant thereof having at least about 80% sequence identity to the amino acid sequence of SEQ ID NO: 50. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 37 or a variant thereof having at least about 80% sequence identity to the amino acid sequence of SEQ ID NO: 37, and a V_{L} comprising the amino acid sequence of SEQ ID NO: 50 or a variant thereof having at least about 80% sequence identity to the amino acid sequence of SEQ ID NO: 50. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 32 or a variant thereof having at least about 80% sequence identity to the amino acid sequence of SEQ ID NO: 32, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 33 or a variant thereof having at least about 80% sequence identity to the amino acid sequence of SEQ ID NO: 33, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 34 or a variant thereof having at least about 80% sequence identity to the amino acid sequence of SEQ ID NO: 34, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 35 or a variant thereof having at least about 80% sequence identity to the amino acid sequence of SEQ ID NO: 35, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 36 or a variant thereof having at least about 80% sequence identity to the amino acid sequence of SEQ ID NO: 36, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 37 or a variant thereof having at least about 80% sequence identity to the amino acid sequence of SEQ ID NO: 37, and a V_{L} comprising the amino acid sequence of SEQ ID NO: 51 or a variant thereof having at least about 80% sequence identity to the amino acid sequence of SEQ ID NO: 51. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 32 or a variant thereof having at least about 80% sequence identity to the amino acid sequence of SEQ ID NO: 32, and a V_{L} comprising the amino acid sequence of SEQ ID NO: 52 or a variant thereof having at least about 80% sequence identity to the amino acid sequence of SEQ ID NO: 52. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 34 or a variant thereof having at least about 80% sequence identity to the amino acid sequence of SEQ ID NO: 34, and a V_{L} comprising the amino acid sequence of SEQ ID NO: 52 or a variant thereof having at least about 80% sequence identity to the amino acid sequence of SEQ ID NO: 52. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 35 or a variant thereof having at least about 80% sequence identity to the amino acid sequence of SEQ ID NO: 35, and a V_{L} comprising the amino acid sequence of SEQ ID NO: 52 or a variant thereof having at least about 80% sequence identity to the amino acid sequence of SEQ ID NO: 52. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 36 or a variant thereof having at least about 80% sequence identity to the amino acid sequence of SEQ ID NO: 36, and a V_{L} comprising the amino acid sequence of SEQ ID NO: 52 or a variant thereof having at least about 80% sequence identity to the amino acid sequence of SEQ ID NO: 52. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the anti-FasL antibody provided herein comprises a V_{H} comprising the amino acid sequence of SEQ ID NO: 37 or a variant thereof having at least about 80% sequence identity to the amino acid sequence of SEQ ID NO: 37, and a V_{L} comprising the amino acid sequence of SEQ ID NO: 52 or a variant thereof having at least about 80% sequence identity to the amino acid sequence of SEQ ID NO: 52. In some embodiments, the anti-FasL antibody provided herein is a full-length anti-FasL antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 53. In some embodiments, the heavy chain constant region comprises or consists of the amino acid sequence of SEQ ID NO: 54. In some embodiments, the light chain constant region comprises or consists of the amino acid sequence of SEQ ID NO: 55. In some embodiments, the light chain constant region comprises or consists of the amino acid sequence of SEQ ID NO: 56.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as inflammatory diseases, autoimmune diseases or cancer). In some embodiments, the individual has one or more risk factors associated with one or more diseases or disorders described herein.

The present application in some embodiments provides a method of delivering an anti-FasL antibody (such as any one of the anti-FasL antibodies described herein, e.g., an isolated anti-FasL antibody) to a cell expressing FasL on the surface in an individual, the method comprising administering to the individual a composition comprising the anti-FasL antibody.

Many diagnostic methods for inflammatory diseases, autoimmune diseases or cancer or any other disease that exhibits abnormalities in the FasL-Fas signaling pathway and the clinical delineation of those diseases are known in the art. Such methods include, but are not limited to, e.g., immunohistochemistry, PCR, and fluorescent in situ hybridization (FISH).

In some embodiments, the anti-FasL antibodies (e.g., full-length anti-FasL antibodies) and/or compositions of the application are administered in combination with a second, third, or fourth agent (including, ag., immunosuppressive agents, anti-inflammatory drugs, anti-tumor agents, growth inhibitors, cytotoxic agents, chemotherapy agents or vascular suppressants) to treat diseases associated with FasL-Fas signaling pathway.

### Dosing and Method of Administering the Anti-FasL Antibodies

The dose of the anti-FasL antibody (such as isolated anti-FasL antibody) compositions administered to an individual (such as a human) may vary with the particular composition, the mode of administration, and the type of disease being treated. In some embodiments, the amount of the composition (such as composition comprising isolated anti-FasL antibody) is effective to result in an objective response (such as a partial response or a complete response) in the treatment of inflammatory diseases, autoimmune diseases or cancer. In some embodiments, the amount of the anti-FasL antibody composition is sufficient to result in a complete response in the individual. In some embodiments, the amount of the anti-FasL antibody composition is sufficient to result in a partial response in the individual. In some embodiments, the amount of the anti-FasL antibody composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-FasL antibody composition.

In some embodiments, the amount of the composition (such as composition comprising isolated anti-FasL antibody) is sufficient to control symptoms and reduce the risk of exacerbations of the individual. In some embodiments, the amount of the composition is sufficient to control symptoms and reduce the risk of exacerbations of the individual. In some embodiments, the amount of the composition (for example when administered along) is sufficient to produce clinical benefit of more than about any of 50%, 60%, 70%, or 77% among a population of individuals treated with the anti-FasL antibody composition.

In some embodiments, the amount of the composition (such as composition comprising isolated anti-FasL antibody), alone or in combination with a second, third, and/or fourth agent, is an amount sufficient to control symptoms and reduce the risk of exacerbations in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-FasL antibody (such as a full-length anti-FasL antibody) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-FasL antibody (such as a full-length anti-FasL antibody) in the composition is included in a range of about 0.001 μg to about 1000 μg.

In some embodiments of any of the above aspects, the effective amount of anti-FasL antibody (such as a full-length anti-FasL antibody) in the composition is in the range of about 0.1 μg/kg to about 100 mg/kg of total body weight.

The anti-FasL antibody compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal or transdermal. In some embodiments, sustained continuous release formulation of the composition may be used. In some embodiments, the composition is administered inhaled. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion. In some embodiments, the administration is to an injection site distal to a first disease site.

### Articles of Manufacture and Kits

In some embodiments of the application, there is provided an article of manufacture containing materials useful for the treatment of disease associated with FasL-Fas signaling pathway, (e.g., inflammatory diseases, autoimmune diseases or cancer) or for delivering an anti-FasL antibody (such as a full-length anti-FasL antibody) to a cell expressing FasL on the surface of the individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-FasL antibody of the application. The label or package insert indicates that the composition is used for treating the particular condition. The label or package insert will further comprise instructions for administering the anti-FasL antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating disease associated with FasL-Fas signaling pathway (such as inflammatory diseases, autoimmune diseases or cancer). In some embodiments, the package insert indicates that the composition is used for treating disease or condition selected from the group consisting of pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, and cancer.

Additionally, the article of manufacture may further comprise a second container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

Kits are also provided that are useful for various purposes, e.g., for treatment of disease associated with FasL-Fas signaling pathway (e.g., inflammatory diseases, autoimmune diseases or cancer), or for delivering an anti-FasL antibody (such as a full-length anti-FasL antibody) to a cell expressing FasL on the surface of the individual, optionally in combination with the articles of manufacture. Kits of the application include one or more containers comprising anti-FasL antibody composition (or unit dosage form and/or article of manufacture) and in some embodiments, further comprise another agent (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-FasL antibody (such as a full-length anti-FasL antibody). In some embodiments, the kit comprises a) a composition comprising any one of the anti-FasL antibodies described herein, and b) an effective amount of at least one other agent, wherein the other agent enhances the effects (e.g., treatment effect, detecting effect) of the anti-FasL antibody. In some embodiments, the kit comprises a) a composition comprising any one of the anti-FasL antibodies described herein, and b) instructions for administering the anti-FasL antibody composition to an individual for treatment of a disease associated with FasL-Fas signaling pathway (e.g., inflammatory diseases, autoimmune diseases or cancer). In some embodiments, the kit comprises a) a composition comprising any one of the anti-FasL antibodies described herein, b) an effective amount of at least one other agent, wherein the other agent enhances the effect (e.g., treatment effect, detecting effect) of the anti-FasL antibody, and c) instructions for administering the anti-FasL antibody composition and the other agent(s) to an individual for treatment of a disease associated with FasL-Fas signaling pathway (e.g., inflammatory diseases, autoimmune diseases or cancer. The anti-FasL antibody and the other agent(s) can be present in separate containers or in a single container. For example, the kit may comprise one distinct composition or two or more compositions wherein one composition comprises an anti-FasL antibody and another composition comprises another agent.

In some embodiments, the kit comprises a nucleic acid (or a set of nucleic acids) encoding an anti-FasL antibody (such as a full-length anti-FasL antibody). In some embodiments, the kit comprises a) a nucleic acid (or a set of nucleic acids) encoding an anti-FasL antibody, and b) a host cell for expressing the nucleic acid (or a set of nucleic acids). In some embodiments, the kit comprises a) a nucleic acid (or a set of nucleic acids) encoding an anti-FasL antibody, and b) instructions for i) expressing the anti-FasL antibody in a host cell, ii) preparing a composition comprising the anti-FasL antibody, and iii) administering the composition comprising the anti-FasL antibody to an individual for the treatment of a disease associated with FasL-Fas signaling pathway (e.g., inflammatory diseases, autoimmune diseases or cancer. In some embodiments, the kit comprises a) a nucleic acid (or a set of nucleic acids) encoding an anti-FasL antibody, b) a host cell for expressing the nucleic acid (or a set of nucleic acids), and c) instructions for i) expressing the anti-FasL antibody in the host cell, ii) preparing a composition comprising the anti-FasL antibody, and iii) administering the composition comprising the anti-FasL antibody to an individual for the treatment of a disease associated with FasL-Fas signaling pathway (e.g., inflammatory diseases, autoimmune diseases or cancer).

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-FasL antibody compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-FasL antibody (such as a full-length anti-FasL antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-FasL antibody and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

### Example 1: Generation of Antigen

### Generation of Recombinant FasL/Fas Antigen

cDNA encoding protein extracellular region of human FasL (h FasL; GenBank ID: 356) (synthesized by sino biological Inc.,) was subcloned into eukaryotic expression vector. His-tag and/or human Fc-tag and/or other conventionally used tags were added to the terminal of cDNA described above, and plasmids containing human FasL extracellular region encoding sequences were constructed. The plasmids were transfected into 293F cells or E. coli to express and produce fusion proteins containing FasL extracellular regions, which named such as His-hFasL, hFc-hFasL respectively, wherein “His” stands for His tag, “hFc” stands for human Fc fragment.

Recombinant fusion proteins with His-tag were purified using Ni-HisCap Smart 6FF 5 ml preloaded column (Smart-Lifesciences, SA036C15) for immobilized metal affinity chromatography (IMAC) analysis according to manufacturer's protocol. Specifically, the cartridges were first equilibrated with buffer Al (50 mM Na₃PO₄, 0.15M NaCl, pH 7.2) with a flow rate of 150 cm/h. The solution containing fusion protein (such as cell culture supernatant), whose pH was adjusted to 7.2, flowed through the cartridges at room temperature (RT) with a flow rate of 150 cm/h. Next, 6×column volumes of buffer Al was used to equilibrate the cartridges at the flow rate of 150 cm/h. 10×column volumes of 50 mM PB solution (containing 0.15M NaCl and 0.2M Imidazole, pH 7.2) was used to wash the cartridges and the eluent was collected. The purified protein was concentrated using an ultrafiltration tube and the solvent of the purified protein was replaced with PBS.

Recombinant fusion proteins with Fc fragment were purified using protein A-MabCap At 4FF 5 ml preloaded column (Smart-Lifesciences, SA023C15). Specifically, the cartridges were first equilibrated with PBS (containing 50 mM PBS and 0.15M NaCl, pH 7.2) with a flow rate of 5 ml/min. The culture supernatant, whose pH was adjusted to 7.2, flowed through the cartridges at room temperature (RT) with a flow rate of 5 ml/min. Next, 6-10×column volumes of PBS was used to equilibrate the cartridges at the flow rate of 5 ml/min. After complete equilibrium, eluent (containing 0.1M Gly and 150 mM NaCl, pH3.2) was added for elution and eluent was collected. The purified protein was concentrated using an ultrafiltration tube and the solvent of the purified protein was replaced with PBS.

### Example 2: Selection of Anti-FasL Single-Chain Antibodies (scFv)

Construction of scFv Antibody Phage Display Library:

Mice were immunized with the extracellular region of human FasL and equal-volume (v/v) adjuvant. After immunization, serum was collected from each immunized mouse and analyzed for IgG titer by ELISA. After several rounds of immunization, spleen was used to establish a phage library. Briefly, RNAs were extracted from spleen of the immunized mice and then reverse-transcribed into cDNAs. V_{H} and V_{K} fragments were amplified using V_{H}- and V_{K}-specific primers. Upon gel extraction and purification, scFvs were generated by linking V_{H} and V_{K}, and were cloned into the phage display plasmid, which were then electroporated into TG1 competent cells, and the scFv antibody phage display library generated by infecting TG1 with phage.

Selection of Anti-FasL Single-Chain Antibodies (scFv):

ScFvs which specifically bound to human hFasL were isolated from the phage display library. Briefly, scFv phage library at 2×10¹¹ PFU was added to an ELISA plate coated with His-hFasL, which incubated at 37° C. for 2 h. After washing 8-15 times (the times of washing increased with increasing number of selection rounds) with PBST solution, the phages binding with hFasL were captured by the FasL antigen on the ELISA plate and the unbound phages were washed off. The phages specifically binding to human hFasL were eluted with 0.1M Glycine-HCl solution (pH2.2). TG1 cells in exponentially growing phase were infected with those phages. After adding ampicillin and incubating for 1 h, helper phage was added and incubated overnight at 28° C. and 200 rpm shaking. Next day, the culture medium was collected and centrifugated, then, the supernatant was harvested and subjected to the next round of screening until the positive scFv antibody library was obtained.

### Selection of Anti-FasL Single-Chain Antibodies Using ELISA:

The phages enriched from the previous panning were screened by ELISA. Briefly, His-hFasL was dissolved in PBS and coated on 96-well plates with 0.1 μg/well followed by incubation at 4° C. overnight. The 96-well plates were washed with PBST and 90 μL of PBS containing 4% skim milk was added to each well. 10 μL of supernatant containing phage-scFv was added to the corresponding well and incubated for 1-2h at 37° C. After washing with PBST solution, the M13-HRP antibody (Sino Biological, 11973-MM05T-H) diluted at 1:4000 was added with 100 μL/well and incubated for 1 h at 37° C. After washing six times with PBST, 100 μL of TMB was added to each well. The plates were incubated for 10 min at room temperature avoiding light contact. The development reaction was terminated using 2M H₂SO₄. Plates were read for the absorbance at 450 nm using a microplate reader. A panel of positive scFv antibodies were obtained and sequenced at the end of screening.

### Example 3: Generation and Characterization of Full-Length Anti-FasL Chimeric Antibodies

### 3.1 Generation of Full-Length Anti-FasL Chimeric Antibodies

The positive scFv antibodies were reformatted as chimeric antibody molecules with a human IgG1 or IgG4 heavy chain constant domain, and a human kappa (κ) light chain constant domain. V_{H} and V_{L} were amplified from the phage expression vectors and introduced into eukaryotic expression vectors pTa1-L (containing a constant domain) and pTT5-H1 (containing IgG1 heavy chain constant domain), or pTT5-H4 (containing IgG4 heavy chain constant domain), respectively. Plasmid expressing the light chain and plasmid expressing the heavy chains were extracted and used to co-transfect 293F cells. After the cells were cultured at 37° C., 5% CO₂ and 120 rpm for 6 days, the culture media was purified using Protein A affinity chromatography. Briefly, the antibody purification using Protein A column is the same as that used in Example 1. The concentration of antibody was determined after ultrafiltration and then subjected to further biochemical and biological analysis.

### 3.2 Determination of Anti-FasL Antibodies Binding Activity

### Affinity of Anti-FasL Antibodies:

The constructed full-length anti-FasL chimeric antibody (reformatted as human IgG4) were subjected to binding assays with human FasL to reflect the binding activity of anti-FasL antibody and human FasL antigen. Briefly, the hFc-hFasL antigen was first dissolved in PBS and coated on 96-well plates with 0.1 μg/well followed by incubation at 4° C. overnight. The 96-well plates were blocked with 5% milk for 1 h at 37° C. and washed with PBST for 6 times. Firstly, each antibody sample was diluted to 66.67 nM, and then serially diluted at a ratio of 1:4. 100 μL of serially diluted antibodies were added to each well and incubated for 1 h at 37° C. and washed with PBST for 6 times. 100 μL of the anti-human kappa-HRP (SouthernBiotech, E1920-MJ11B, 1:4000 dilution) was added to each well and incubated for 1 h at 37° C. After washing with PBST for 6 times, TMB was added with 100 μL/well and incubated for 10-20 minutes at 37° C. 2M H₂SO₄ was used to stop the reaction. Absorbance at 450 nm was read using a microplate reader. The binding curves were generated by Graphpad Prism and the EC₅₀ of each anti-FasL antibody was calculated.

As shown in Table 5, the selected anti-FasL chimeric antibodies FL-M05, FL-M08, FL-M54, FL-M56, FL-M78, FL-M88, FL-M89 all had well binding ability to human FasL antigen. The binding ability of the above anti-FasL antibodies to FasL was significantly better than the FasL antagonist APG101 (Fas fusion protein, Apogenix), and the binding ability of the above anti-FasL antibodies was better than or comparable to the reference antibody MAB126-100 (commercial anti-FasL antibody, RD).

TABLE 5
The binding ability of anti-FasL chimeric antibody to human FasL

| Antibody Name | EC₅₀(nM) |
|---|---|
| FL-M05 | 0.186 |
| FL-M08 | 0.157 |
| FL-M54 | 0.725 |
| FL-M56 | 0.661 |
| FL-M78 | 0.811 |
| Fl-M88 | 0.539 |
| Fl-M89 | 0.441 |
| APG101 | 23.530 |
| MAB126-100 | 0.948 |

Anti-FasL antibodies neutralize the binding of ligand and receptor: The assay was used to identify the ability of anti-FasL chimeric antibodies (reformatted as human IgG4) to neutralize the binding of ligand FasL and its receptor Fas. Briefly, the His-hFas antigen prepared in example 1 was coated on 96-well plates with 0.1 μg/well followed by incubation at 4° C. overnight. The 96-well plates were blocked with 2% milk for 1 h at 37° C. and washed with PBST. Firstly, each anti-FasL antibody sample was diluted to 12.0 μg/ml, and then serially diluted at a ratio of 1:4. The hFc-hFasL antigen prepared in example 1 (1.25 ug/mL) was mixed with serially diluted anti-FasL antibodies and pre-mixed at 37° C. for 30 min. The antigen-antibody premix was added to 96-well plate, 100 L/well, and incubated at 37° C. for 1 h. 0 WIL of the anti-human IgG1 Fc-AP (SouthernBiotech, 9054-04, 1:3000 dilution) was added to each well and incubated for 1 h at 37° C. After washing with PBST for 6 times, pNPP was added with 100 L/well and incubated for 10-20 minutes at 37° C. Absorbance at 405 nm was read using a microplate reader. The neutralization curves were generated by Graphpad Prism.

The results showed that, taking FL-M05 and FL-M08 as examples, the ability of anti-FasL chimeric antibody to neutralize the binding of FasL and Fas was comparable to the reference antibody MAB126-100 (results not shown).

### 3.3 Determination of Anti-FasL Antibodies Activity

### Anti-FasL Antibody Inhibited FasL Antigen-Induced Apoptosis of Jurkat Cells:

When actinomycin D was added to inhibit cell division, exogenous FasL could activate apoptosis signaling pathway and mediate apoptosis. The amount of ATP in cells was directly proportional to the number of cells in the culture. Based on the above principles, the amount of ATP in cells is used to reflect the apoptosis level, and then the activity of anti-FasL chimeric antibody (reformatted as human IgG4) to inhibit FasL antigen-induced apoptosis of Jurkat cells was determined.

Jurkat cells at logarithmic growth stage (purchased from the cell bank of Peking Union Medical College, 1101HUM-PUMC000075) were inoculated into 96-well plates with 5×10⁴ cells/wells. Each antibody sample was diluted to 6.667 nM and then serially diluted at a ratio of 1:3. 50 μL actinomycin D (Hanhui-pharma, H20023504), 25 μL hFc-FasL recombinant fusion protein and 25 μL diluted anti-FasL antibody were added to a 96-well plate containing Jurkat cells. The final concentrations of actinomycin D and FasL recombinant fusion protein were 32 ng/mL and 5 ng/mL, respectively. The cells were cultured at 37° C., 5% CO₂ for 24 h. The amount of ATP was measured using the CellTiter-Glo® luminescent cell viability assay kit (Promega, G7572), which was proportional to the generated luminescence signal. That is, CellTiter-Glo® reagent was added to 96-well cell culture plates with 50 μL/well, which incubated at room temperature for 10 min after mixing. The fluorescence values were read using a microplate reader. The curve was generated by GraphPad Prism software, and the IC₅₀ value of each anti-FasL antibody was calculated.

As shown in Table 6, anti-FasL chimeric antibodies FL-M05, FL-M08, FL-M54, FL-M56, FL-M78, FL-M88, FL-M89 all had significant inhibitory effects on FasL antigen-induced apoptosis of Jurkat cells. The ability of the above anti-FasL antibodies inhibiting FasL antigen-induced apoptosis of Jurkat cells were significantly better than the reference APG101 and comparable to the reference antibody 119-4A (anti-FasL antibody, Apogenix).

TABLE 6
The anti-FasL chimeric antibodies inhibiting FasL
antigen-induced apoptosis of Jurkat cells

| Antibody Name | IC₅₀(nM) |
|---|---|
| FL-M05 | 0.109 |
| FL-M08 | 0.035 |
| FL-M54 | 0.018 |
| FL-M56 | 0.020 |
| FL-M78 | 0.023 |
| Fl-M88 | 0.020 |
| Fl-M89 | 0.023 |
| APG101 | >100 |
| 119-4A | 0.033 |

### Anti-FasL Antibody Inhibited FasL Antigen-Induced Apoptosis of HepG2 Cells:

The experimental principle is the same as above. The amount of ATP in cells is used to reflect the apoptosis level, and then the activity of anti-FasL chimeric antibody (reformatted as human IgG4) to inhibit FasL antigen-induced apoptosis of HepG2 cells was determined.

HepG2 cells at logarithmic growth stage (purchased from the cell bank of Peking Union Medical College, 1101HUM-PUMC0000035) were inoculated into 96-well plates with 2×10⁴ cells/wells. Each antibody sample was diluted to 6.667 nM and then serially diluted at a ratio of 1:3. 50 μL actinomycin D, 25 μL hFc-FasL recombinant fusion protein and 25 μL diluted anti-FasL antibody were added to a 96-well plate containing HepG2 cells. The final concentrations of actinomycin D and hFc-FasL recombinant fusion protein were 1 μg/mL and 5 ng/mL, respectively. The cells were cultured at 37° C., 5% CO₂ for 48 h. The amount of ATP was measured using the CellTiter-Glo® luminescent cell viability assay kit (Promega, G7572), which was proportional to the generated luminescence signal. That is, CellTiter-Glo® reagent was added to 96-well cell culture plates with 50 L/well, which incubated at room temperature for 10 min after mixing. The fluorescence values were read using a microplate reader. The curve was generated by GraphPad Prism software, and the IC₅₀ value of each anti-FasL antibody was calculated.

As shown in Table 7, anti-FasL chimeric antibodies FL-M05, FL-M08, FL-M54, FL-M56, FL-M78, FL-M88, FL-M89 all had significant inhibitory effects on FasL antigen-induced apoptosis of HepG2 cells. The ability of the above anti-FasL antibodies inhibiting FasL antigen-induced apoptosis of HepG2 cells were significantly better than the reference APG101 and MAB126-100, and also better than or comparable to the reference antibody 119-4A.

TABLE 7
The anti-FasL chimeric antibodies inhibiting
FasL antigen-induced apoptosis of HepG2 cells

| Antibody Name | IC₅₀(nM) |
|---|---|
| FL-M05 | 0.030 |
| FL-M08 | 0.016 |
| FL-M54 | 0.018 |
| FL-M56 | 0.020 |
| FL-M78 | 0.016 |
| Fl-M88 | 0.019 |
| Fl-M89 | 0.021 |
| APG101 | 11.300 |
| 119-4A | 0.029 |
| MAB126-100 | 0.099 |

### Anti-FasL Antibody Inhibited FasL Antigen-Induced NF-κB Signaling Pathway Activation:

When actinomycin D was added to inhibit cell division, exogenous FasL could activate the NF-κB signaling pathway in addition to apoptosis signaling pathway. After blocking the apoptotic pathway with Caspase inhibitors, exogenous FasL could only induce NF-κB signaling pathway activation. HepG2-Dual (InvivoGen, hepg2d-nfis) was a dual reporter cell line of NF-κB-SEAP and IRF-Lucia, in which exogenous FasL induced the activation of the NF-κB pathway to initiate the expression of Secreted Embryonic Alkaline Phosphatase (SEAP). It could be determined by QUANTI-Blue™ Substrate conversion. Based on the above experimental principles, the activity of an anti-FasL chimeric antibody (reformatted as human IgG4) to inhibit activation of the NF-κB signaling pathway induced by FasL antigen was determined by SEAP activity in HepG2-Dual.

HepG2-Dual cells at logarithmic growth stage were inoculated into 96-well plates with 1×10⁵ cells/wells. Each antibody sample was diluted to 625 nM followed by gradient dilution. hFc-FasL (final concentration of 700 ng/ml), actinomycin D, Caspase inhibitor Z-VAD-FMK (Beyotime, C1202-5 mg) and gradient diluted anti-FasL antibody were added to a 96-well plate containing HepG2-Dual cells. The cells were cultured at 37° C., 5% CO₂ for 24h. 40 μL cell culture supernatant was mixed with 160 IL preheated QUANTI-Blue™ (Invivogen), incubated for 90 minutes, and absorbance at 650 nm was read. The curve was generated by GraphPad Prism software, and the EC₅₀ value of each anti-FasL antibody was calculated.

As shown in Table 8, anti-FasL chimeric antibodies FL-M05, FL-M08, FL-M54, FL-M56, FL-M78, FL-M88, FL-M89 all had significant inhibitory effects on FasL antigen-induced NF-κB signaling pathway activation. The ability of the above anti-FasL antibodies inhibiting FasL antigen-induced NF-κB signaling pathway activation were significantly better than the reference APG101.

TABLE 8
The anti-FasL chimeric antibodies inhibiting FasL
antigen-induced NF-κB signaling pathway activation

| Antibody Name | EC₅₀(nM) |
|---|---|
| FL-M05 | 1.132 |
| FL-M08 | 0.704 |
| FL-M54 | 1.973 |
| FL-M56 | 2.727 |
| FL-M78 | 3.011 |
| Fl-M88 | 1.468 |
| Fl-M89 | 1.327 |
| APG101 | 7.226 |

### Anti-FasL Antibody Inhibited Jurkat Cell Self-Killing Induced by OKT (CD3 Antibody):

OKT could induce the activation of Jurkat cells and the up-regulation of the expression of natural FasL, and under the combined action of cytokine IL-2, the apoptosis signal pathway of Jurkat cells was activated, so that the Jurkat cells could generate self-killing activity. The cleavage of Caspase 3/7 was a sign of apoptosis. Based on the above principles, the degree of apoptosis could be determined by detecting the amount of activated Caspase3/7, and then the activity of anti-FasL chimeric antibody (reconstructed adult IgG4 form) to inhibit OKT-induced Jurkat cell self-killing could be determined.

OKT3 (Biolegend, 317302) was coated in 96-well plates at 0.1 μg/well at 4° C. overnight. Jurkat cells were inoculated into a 96-well plate, 7.5×10⁴ cells/well. At the same time, actinomictin D, IL2 (Beijing SL Pharm, S19991009) and anti-FasL antibody (FL-M54, FL-M78, FL-M88 or FL-M89) were added into the 96-well plate, where the final concentration of anti-FasL antibody FL-M54 was 60 nM or 180 nM. The final concentration of FL-M78, FL-M88 and FL-M89 was 40 nM. The final concentration of actinomycin D was 32 ng/ml and the final concentration of IL2 was 5001U/ml. Cultured at 3TC, 50% CO₂ for 24 hours.

Caspase-Glo® 3/7 detection kit (Promega, G8093) was used to detect the amount of activated Caspase3/7. After adding Caspase-Glo® 3/7 reagent, the intensity of the fluorescence signal generated was proportional to the activity of Caspase-3/7. Briefly, Caspase-Glo® 3/7 reagent was added to the 96-well plate with 50 μL/well, and incubated for 10 min. The fluorescence value was read using a microplate reader.

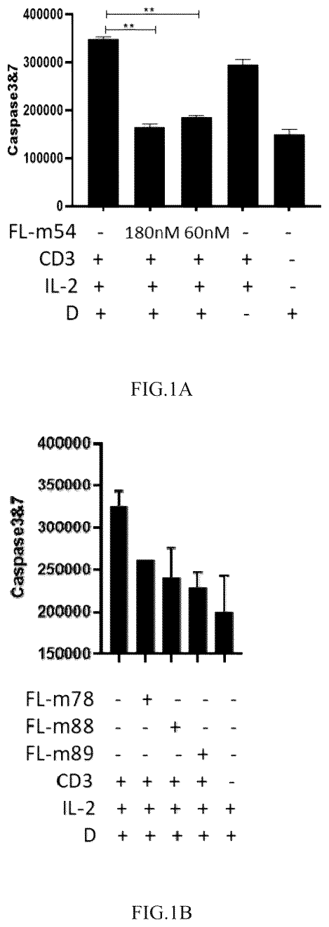

As shown in FIGS. 1A-1B, the anti-FasL chimeric antibodies FL-M54, FL-M78, FL-M88 and FL-M89 all inhibited Caspase3/7 production, indicating that the above anti-FasL antibodies were effective in inhibiting the OKT-induced self-killing of Jurkat cells.

### Example 4: Humanization of Anti-FasL Antibodies

Based on the typical structure of V_{H}/V_{L} CDR of the mouse antibody FL-M54, FL-M78 and FL-M88 respectively, heavy and light chain variable region sequences were aligned with the sequences in antibody Germline database to obtain human germline templates of high homology. The CDR regions of the mouse antibody were grafted into selected human germline templates to produce humanized variable regions, which were then recombined with corresponding human IgG constant regions (preferably IgG4 heavy chain and κ light chain). The embedded residues, the residues that interact directly with the CDR regions, and the residues that have an important effect on the conformation of V_{L} and V_{H} were then reverse-mutated based on the three-dimensional structure of the mouse antibody and different light chains and heavy chains can be designed and combined to obtain a series of humanized molecules.

Finally, humanized antibodies of FL-M54 were obtained six, named humFL-M54-1-humFL-M54-15, respectively; humanized antibodies of FL-M78 were obtained sixteen, named humFL-M78-1-humFL-M78-4, respectively; humanized antibodies of FL-M88 were obtained sixteen, named humFL-M88-1-humFL-M88-18, respectively.

### Example 5: Characterization of the Humanized Anti-FasL Antibodies

Activity of the FL-M54, FL-M78, FL-M88 humanized anti-FasL antibodies were tested according to the corresponding protocol as described in example 3, including affinity assays with hFasL, and inhibition of FasL antigen induced HepG2 or Jurkat cell apoptosis assay, inhibition of NF-κB pathway activation assay, and inhibition of OKT3 induced Jurkat cell self-killing assay.

### 5.1 Affinity of Anti-FasL Antibodies:

Using the corresponding protocol described in Example 3.2, the binding activity of FL-M54, FL-M78 and FL-M88 humanized anti-FasL antibodies (reformatted as human IgG4) to human FasL antigen was determined.

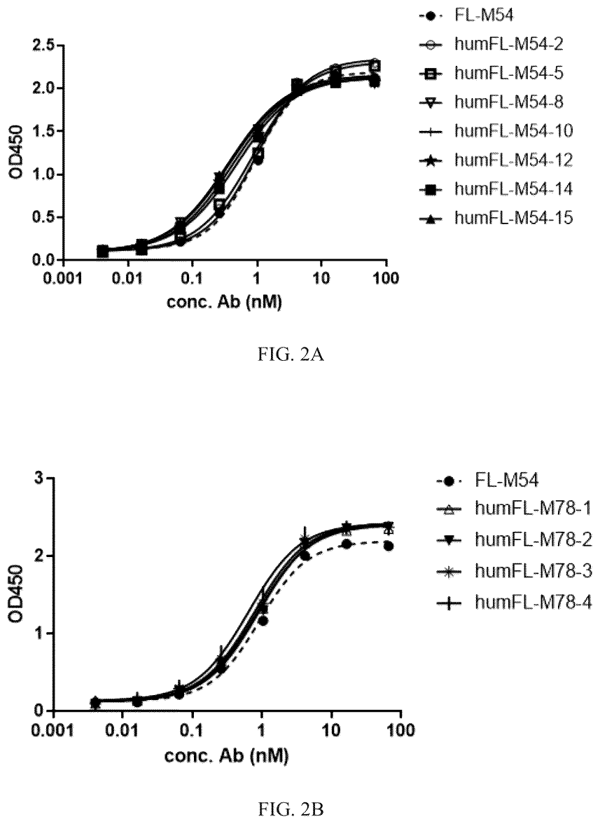

As shown in FIG. 2A, FL-M54 humanized antibodies humFL-M54-2, humFL-M54-5, humFL-M54-8, humFL-M54-10, humFL-M54-12, humFL-M54-14, humFL-M54-15 all had well binding activity to human FasL antigen, and its binding ability was better than or comparable to the chimeric antibody FL-M54.

As shown in FIG. 2B, FL-M78 humanized antibodies humFL-M78-1-humFL-M78-4 all had well binding activity to human FasL antigen, and its binding ability was better than the chimeric antibody FL-M54.

As shown in Table 9, FL-M88 humanized antibodies humFL-M88-1, humFL-M88-3-humFL-M88-7, humFL-M88-9-humFL-M88-13, humFL-M88-15-humFL-M88-18 all had well binding activity to human FasL antigen, and its binding ability was better than the chimeric antibody FL-M88 or the reference antibody 119-4A.

TABLE 9
The binding ability of FL-M88 humanized
antibodies to human FasL

| Antibody Name | EC₅₀(nM) |
|---|---|
| humFL-M88-1 | 0.2650 |
| humFL-M88-3 | 0.2732 |
| humFL-M88-4 | 0.2880 |
| humFL-M88-5 | 0.2302 |
| humFL-M88-6 | 0.2353 |
| humFL-M88-7 | 0.3396 |
| humFL-M88-9 | 0.2839 |
| humFL-M88-10 | 0.2837 |
| humFL-M88-11 | 0.3433 |
| humFL-M88-12 | 0.2761 |
| humFL-M88-13 | 0.3016 |
| humFL-M88-15 | 0.3013 |
| humFL-M88-16 | 0.3718 |
| humFL-M88-17 | 0.3293 |
| humFL-M88-18 | 0.2716 |
| FL-M88 | 0.5391 |
| 119-4A | 0.5247 |

### 5.2 Determination of Activity of Humanized Anti-FasL Antibody:

### Anti-FasL Antibody Inhibited FasL Antigen-Induced Cell Apoptosis:

Using the corresponding protocol described in Example 3.3, the activity of FL-M54, FL-M78, FL-M88 humanized anti-FasL antibodies (reformatted as human IgG4) to inhibit FasL antigen-induced apoptosis of Jurkat or HepG2 cells was determined.

As shown in Table 10, humanized antibodies humFL-M54-1˜humFL-M54-15 all had significant inhibitory effects on FasL antigen-induced apoptosis of Jurkat or HepG2 cells. In comparison, the ability of the above FL-M54 humanized antibodies inhibiting FasL antigen-induced apoptosis of Jurkat or HepG2 were comparable to the parental chimeric antibody FL-M54, and its ability inhibiting FasL antigen-induced apoptosis of Jurkat or HepG2 were better than or comparable to the reference antibody MAB126-100.

TABLE 10
FL-M54 humanized antibodies inhibiting
FasL antigen-induced cell apoptosis

| | Jurkat | HepG2 |
|---|---|---|
| Antibody Name | IC₅₀(nM) | IC₅₀(nM) |
| humFL-M54-1 | 0.035 | 0.028 |
| humFL-M54-2 | 0.028 | 0.031 |
| humFL-M54-3 | / | 0.035 |
| humFL-M54-4 | 0.053 | 0.030 |
| humFL-M54-5 | 0.052 | 0.027 |
| humFL-M54-6 | / | 0.031 |
| humFL-M54-7 | 0.059 | 0.028 |
| humFL-M54-8 | 0.044 | 0.025 |
| humFL-M54-9 | 0.035 | 0.026 |
| humFL-M54-10 | 0.017 | 0.034 |
| humFL-M54-11 | 0.037 | 0.028 |
| humFL-M54-12 | 0.015 | 0.032 |
| humFL-M54-13 | 0.028 | 0.029 |
| humFL-M54-14 | 0.038 | 0.023 |
| humFL-M54-15 | 0.015 | 0.030 |
| FL-M54 | 0.023 | 0.023 |
| MAB126-100 | 0.046 | 0.074 |

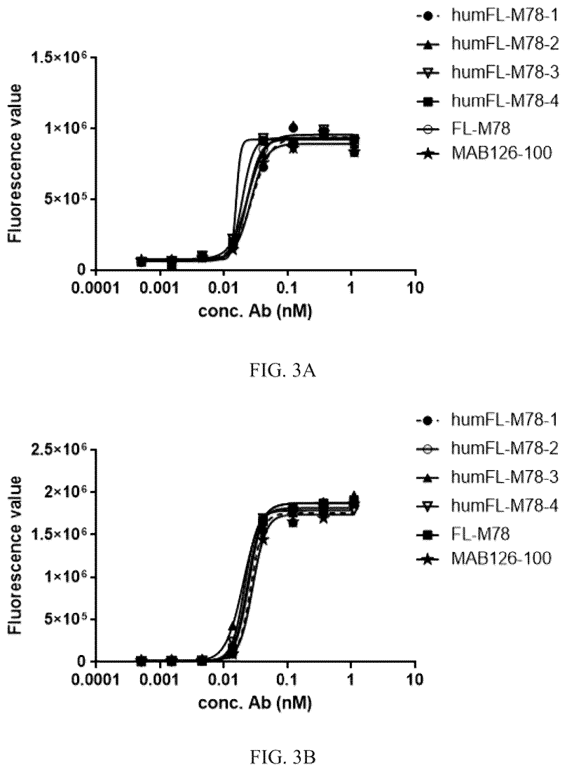

As shown in FIGS. 3A-3B, humanized antibodies humFL-M78-1-humFL-M78-4 all had significant inhibitory effects on FasL antigen-induced apoptosis of Jurkat (FIG. 3A) or HepG2 (FIG. 3B) cells. The ability of the above FL-M78 humanized antibodies inhibiting FasL antigen-induced apoptosis of Jurkat or HepG2 were better than or comparable to the parental chimeric antibody FL-M78 and the reference antibody MAB126-100.

As shown in Table 11, humanized antibodies humFL-M88-1, 3, 4, 5, 6, 7, 9, 10, 12, 13, 15, 18 all had significant inhibitory effects on FasL antigen-induced apoptosis of Jurkat or HepG2 cells. The ability of the above FL-M88 humanized antibodies inhibiting FasL antigen-induced apoptosis of Jurkat or HepG2 were comparable to the parental chimeric antibody FL-M88, and better than or comparable to the reference antibody 119-4A.

TABLE 11
FL-M88 humanized antibodies inhibiting
FasL antigen-induced cell apoptosis

| | Jurkat | HepG2 |
|---|---|---|
| Antibody Name | IC₅₀(nM) | IC₅₀(nM) |
| humFL-M88-1 | 0.01929 | 0.01252 |
| humFL-M88-3 | 0.01919 | 0.02762 |
| humFL-M88-4 | 0.02935 | 0.03327 |
| humFL-M88-5 | 0.01865 | 0.02225 |
| humFL-M88-6 | / | 0.02035 |
| humFL-M88-7 | / | 0.02740 |
| humFL-M88-9 | 0.02200 | 0.02246 |
| humFL-M88-10 | 0.02194 | 0.02085 |
| humFL-M88-12 | 0.02340 | / |
| humFL-M88-13 | 0.02426 | 0.01258 |
| humFL-M88-15 | 0.01488 | 0.01435 |
| humFL-M88-18 | 0.03250 | / |
| FL-M88 | 0.01752 | 0.01551 |
| 119-4A | 0.04966 | 0.03693 |

Anti-FasL antibody inhibited FasL antigen-induced NF-κB signaling pathway activation: Using the corresponding protocol described in Example 3.3, the activity of FL-M78, FL-M88 humanized anti-FasL antibodies (reformatted as human IgG4) to inhibit FasL antigen-induced NF-κB signaling pathway activation was determined.

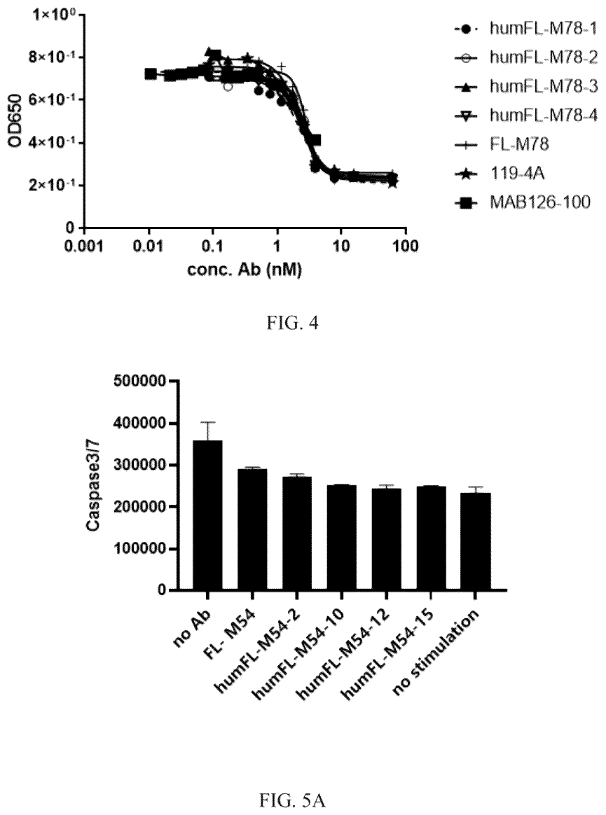

As shown in FIG. 4, humanized antibodies humFL-M78-1˜humFL-M78-4 all had inhibitory effects on FasL antigen-induced NF-κB signaling pathway activation, and the ability of the above FL-M78 humanized antibodies inhibiting FasL antigen-induced NF-κB signaling pathway activation were comparable to the parental chimeric antibody FL-M78, the reference antibody 119-4A and MAB126-100.

As shown in Table 12, humanized antibodies humFL-M88-1, humFL-M88-3, humFL-M88-13, humFL-M88-15 all had inhibitory effects on FasL antigen-induced NF-κB signaling pathway activation, and the ability of the above FL-M78 humanized antibodies inhibiting FasL antigen-induced NF-κB signaling pathway activation were better than or comparable to the parental chimeric antibody FL-M88, and the reference antibody 119-4A.

TABLE 12
FL-M88 humanized antibodies inhibiting FasL
antigen-induced NF-κB signaling pathway activation

| Antibody Name | EC₅₀(nM) |
|---|---|
| humFL-M88-1 | 0.4543 |
| humFL-M88-3 | 0.4438 |
| humFL-M88-13 | 0.6975 |
| humFL-M88-15 | 0.4698 |
| Fl-M88 | 0.9244 |
| 119-4A | 1.063 |

### Anti-FasL Antibody Inhibited Jurkat Cell Self-Killing Induced by OKT (CD3 Antibody):

Using the corresponding protocol described in Example 3, the activity of FL-M54, FL-M78, FL-M88 humanized anti-FasL antibodies (reformatted as human IgG4) to inhibit OKT-induced Jurkat cell self-killing was determined. Wherein only antibody was not added into the no antibody group, and OKT3 and antibody was not added into the no stimulation group.

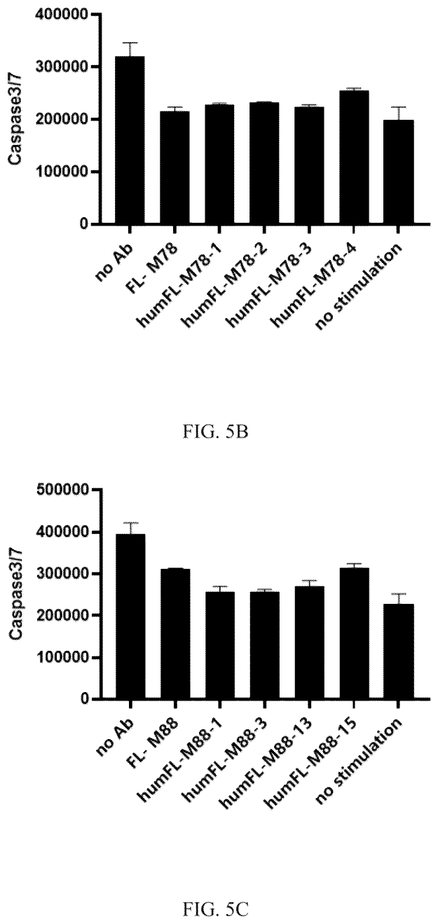

As shown in FIG. 5A, humanized antibodies humFL-M54-2, humFL-M54-10, humFL-M54-12, humFL-M54-15 all inhibited Caspase3/7 production, indicating that the above anti-FasL antibodies could inhibited the OKT-induced self-killing of Jurkat cells, and the ability of the above FL-M54 humanized antibodies inhibiting the OKT-induced self-killing of Jurkat cells were better than or comparable to the parental chimeric antibody FL-M54.

As shown in FIG. 5B, humanized antibodies humFL-M78-1-humFL-M78-4 all inhibited Caspase3/7 production, indicating that the above anti-FasL antibodies could inhibited the OKT-induced self-killing of Jurkat cells, and the ability of the above FL-M78 humanized antibodies inhibiting the OKT-induced self-killing of Jurkat cells were comparable to the parental chimeric antibody FL-M78.

As shown in FIG. 5C, humanized antibodies humFL-M88-1, humFL-M88-3, humFL-M88-13, humFL-M88-15 all inhibited Caspase3/7 production, indicating that the above anti-FasL antibodies could inhibited the OKT-induced self-killing of Jurkat cells, and the ability of the above FL-M88 humanized antibodies inhibiting the OKT-induced self-killing of Jurkat cells were comparable to the parental chimeric antibody FL-M88.

### Anti-FasL Antibody Inhibited PBMC Killing Jurkat Cells:

PBMC cells contain T cells and NK cells. Therefore, PBMC cells could be used to mimic the natural blood immune environment in vivo. Plant hemagglutinin (PHA-P) could activate PBMC cells. In the presence of PHA-P, PBMC co-cultured with Jurkat cells could activate FasL-Fas mediated apoptosis pathway in Jurkat cells and killed it. Based on the above experimental principles, the degree of apoptosis of Jurkat cells could be determined by detecting the amount of activated Caspase3/7, and then the activity of humanized antibodies of FL-M54, FL-M78, FL-M88 (reformatted as human IgG4) inhibiting PBMC killing Jurkat cells were determined.

PBMC cells (TPCS, PB025C, 7.5×10⁴ cells/well) and Jurkat cells (2.5×10⁴ cells/well) were seeded into 96-well plates, and PHA-P (Sigma, L8754) and IL2 and anti-FasL humanized antibody were added simultaneously. The final concentrations of PHA-P, IL2 and antibody were 10 μg/ml, 100 U/ml and 333 nM, respectively. While the control group was set up, wherein only antibody was not added into the no antibody group, and PHA-P and antibody was not added into the no stimulation group. Cultured at 37° C., 5% CO₂ for 3.5 h. Caspase-Glo®3/7 reagent was added into the 96-well plate with 50 μL/well, and incubated for 10 min. The fluorescence values were read using a microplate reader.

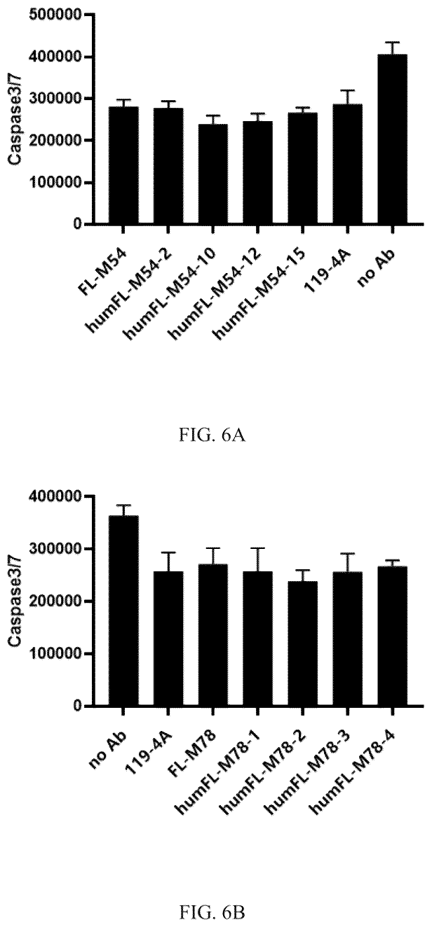

As shown in FIG. 6A, humanized antibodies humFL-M54-2, humFL-M54-10, humFL-M54-12, humFL-M54-15 all could effectively inhibited PBMC killing Jurkat cells, and the inhibitory activity of humFL-M54-10, humFL-M54-12 were better than the parental chimeric antibody FL-M78 or the reference antibody 119-4A, the inhibitory activity of humFL-M54-2, humFL-M54-15 were comparable to the parental chimeric antibody FL-M78 or the reference antibody 119-4A.

As shown in FIG. 6B, humanized antibodies humFL-M78-1˜humFL-M78-4 all could effectively inhibited PBMC killing Jurkat cells, and the inhibitory activity of the above antibodies were comparable to the parental chimeric antibody FL-M78 or the reference antibody 119-4A.

As shown in FIG. 6C, humanized antibodies humFL-M88-1, humFL-M88-3, humFL-M88-13, humFL-M88-15 all could effectively inhibited PBMC killing Jurkat cells, and the activity of humFL-M88-1, humFL-M88-3, humFL-M88-13 inhibiting PBMC killing Jurkat cells were better than the parental chimeric antibody FL-M88, the reference antibody 119-4A and APG101.

### Example 6: Role of Anti-FasL Antibody in Model Mice with Exogenous FasL-Induced Liver Injury

Exogenous FasL could activate the Fas-FasL pathway in mouse hepatocytes, thereby causing hepatocyte death in mice. Dead hepatocytes can release glutamate pyruvate transaminase (ALT) into the blood, and thus ALT is an important indicator reflecting liver injury. Based on the above principle, the degree of liver injury of mice can be reflected by the ALT levels detected in serum.

44 female BALB/C mice (Beijing Vital River Laboratory Animal Technology Co., Ltd., aged 6-8 weeks) were randomly divided into different groups, the experimental group consisted of model control group (no antibody injection group/no antibody group), low-dose group and high-dose group, with 6 mice in each group, and simultaneously set up a blank control group, with 2 mice in each group. Mice in each experimental group were injected with 0.03 mg/kg of hFc-hFasL protein (prepared in example 1) via the tail vein. When injecting hFc-hFasL for 30 minutes, anti-FasL antibody (using humFL-M78-1, humFL-M78-2 as example) or was injected into each experimental group of mice via the tail vein, with a dosage of 0.006 mg/kg in the low-dose group and 0.03 mg/kg in the high-dose group. When injecting hFc-hFasL for 24 hours, 200 μL of blood was taken from the orbital venous plexus of mice and centrifuged at 1500 g at room temperature for 10 min. The serum was collected and ALT level was detected with ALT Detection Kit (Nanjing Jiancheng, C009-2-1).

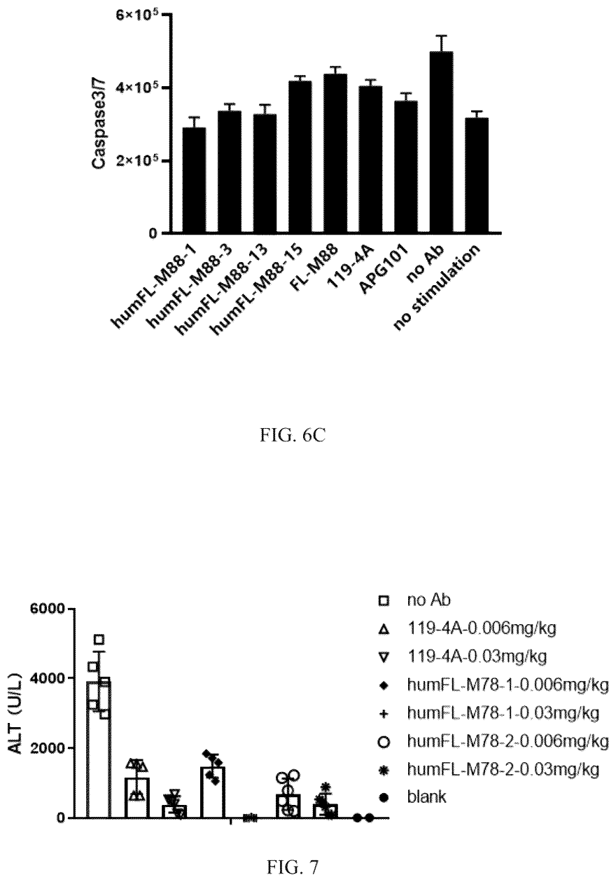

As shown in FIG. 7, the exemplary anti-FasL antibody humFL-M78-1, humFL-M78-2 and the reference antibody 119-4A all significantly inhibited Exogenous FasL-induced ALT elevation in a dose-dependent manner. Moreover, the inhibitory effect of humFL-M78-1 at low doses and humFL-M78-2 at high doses was better than that of 119-4A at corresponding doses. The above results indicated that the anti FasL antibody of this application could play a role in the treatment of liver injury.

### Example 7: Role of Anti-FasL Antibody in a Mouse Model of APAP-Induced Liver Injury

Acetaminophen (APAP) was metabolized through the liver, and its metabolite could form protein adducts with human proteins in hepatocytes. It could also consume glutathione in the body, causing oxidative stress and cytotoxicity. It was a common drug that causes liver damage. The liver cell death directly caused by APAP could lead to innate immune activation or simultaneously trigger adaptive immune activation. Large numbers of immune cells expressed FasL/TRAIL, which in turn promoted more liver cell death. The degree of liver injury of mice can be reflected by the ALT levels detected.

Experiment 1: twenty female BALB/C mice (Beijing Vital River Laboratory Animal Technology Co., Ltd., aged 6-8 weeks) were randomly divided into different groups, the experimental group consisted of model control group (no antibody injection group/no antibody group), low-dose group and high-dose group, with 6 mice in each group, and simultaneously set up a blank control group, with 2 mice in each group. Firstly, all the mice were fasted, but water was allowed. After fasting for about 16h, mice in each experimental group were intraperitoneally injected with 200 mg kg APAP (Aladdin, A105808). When injecting APAP for 30 minutes, anti-FasL antibody (using FL-M88 as an example) was injected into each experimental group of mice through the tail vein, with a dosage of 10 mg/kg in the low-dose group and 20 mg/kg in the high-dose group, meanwhile, the mice of model control group were only given an equal amount of normal saline. When injecting APAP for 24 hours, 200 μL of blood was taken from the orbital venous plexus of mice and centrifuged at 1500 g at room temperature for 10 min. The serum was collected and ALT level was detected with ALT Detection Kit (Nanjing Jiancheng, C009-2-1).

Experiment 2: twenty female BALB/C mice (Beijing Vital River Laboratory Animal Technology Co., Ltd., aged 6-8 weeks) were randomly divided into different groups, the experimental group consisted of model control group (no antibody injection group/no antibody group), and antibody group, with 6 mice in each group, and simultaneously set up a blank control group, with 2 mice in each group. Firstly, all the mice were fasted, but water was allowed. After fasting for about 16h, mice in each experimental group were intraperitoneally injected with 200 mg kg APAP (Aladdin, A105808). When injecting APAP for 30 minutes, anti-FasL antibody (using humFL-M88-1 or humFL-M78-2 as example) with a dosage of 20 mg/kg was injected into each experimental group of mice through the tail vein, meanwhile, the mice of model control group were only given an equal amount of normal saline. When injecting hFc-hFasL for 24 hours, 200 μL of blood was taken from the orbital venous plexus of mice and centrifuged at 1500 g at room temperature for 10 min. The serum was collected and ALT level was detected with ALT Detection Kit (Nanjing Jiancheng, C009-2-1).

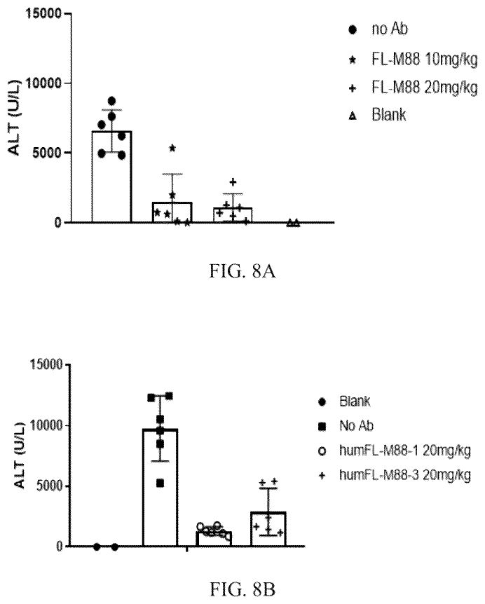

As shown in FIG. 8A-8B, the exemplary anti-FasL antibody FL-M88, humFL-M88-1 and humFL-M88-3 all significantly inhibited APAP-induced ALT elevation. It was suggested again that anti-FasL antibody of this application could play a role in the treatment of liver injury.

What is claimed is:

1-4. (canceled)

5. An isolated antibody that specifically recognizes FasL, comprising:
a heavy chain variable domain (VH), wherein the VH comprising
an HC-CDR1 comprising the amino acid sequence of SEQ ID NO: 2,
an HC-CDR2 comprising the amino acid sequence of SEQ ID NO: 5, and
an HC-CDR3 comprising the amino acid sequence of SEQ ID NO: 8;
and a light chain variable domain (VL), wherein the VL comprising
an LC-CDR1 comprising the amino acid sequence of SEQ ID NO: 11,
an LC-CDR2 comprising the amino acid sequence of SEQ ID NO: 14, and
an LC-CDR3 comprising the amino acid sequence of SEQ ID NO: 17.

6. An isolated antibody that specifically recognizes FasL, comprising:
a VH comprising an HC-CDR1, an HC-CDR2, and an HC-CDR3 of a VH comprising the amino acid sequence of SEQ ID NO: 26;
and a VL comprising an LC-CDR1, an LC-CDR2, and an LC-CDR3 of a VL comprising the amino acid sequence of SEQ ID NO: 43.

7. (canceled)

8. The isolated antibody that specifically recognizes FasL of claim 5, comprising:
(i) a VH comprising the amino acid a VH comprising the amino acid sequence of SEQ ID NO: 26, or a variant thereof having at least about 80% sequence identity to the amino acid sequence of SEQ ID NO: 26; and a VL comprising the amino acid sequence of SEQ ID NO: 43, or a variant thereof having at least about 80% sequence identity to the amino acid sequence of SEQ ID NO: 43;
(ii) a VH comprising the amino acid sequence of SEQ ID NO: 27, or a variant thereof having at least about 80% sequence identity to the amino acid sequence of SEQ ID NO: 27; and a VL comprising the amino acid sequence of SEQ ID NO: 44, or a variant thereof having at least about 80% sequence identity to the amino acid sequence of SEQ ID NO: 44;
(iii) a VH comprising the amino acid sequence of SEQ ID NO: 28, or a variant thereof having at least about 80% sequence identity to the amino acid sequence of SEQ ID NO: 28; and a VL comprising the amino acid sequence of SEQ ID NO: 44, or a variant thereof having at least about 80% sequence identity to the amino acid sequence of SEQ ID NO: 44;
(iv) a VH comprising the amino acid sequence of SEQ ID NO: 27, or a variant thereof having at least about 80% sequence identity to the amino acid sequence of SEQ ID NO: 27; and a VL comprising the amino acid sequence of SEQ ID NO: 45, or a variant thereof having at least about 80% sequence identity to the amino acid sequence of SEQ ID NO: 45; or
(v) a VH comprising the amino acid sequence of SEQ ID NO: 28, or a variant thereof having at least about 80% sequence identity to the amino acid sequence of SEQ ID NO: 28; and a VL comprising the amino acid sequence of SEQ ID NO: 45, or a variant thereof having at least about 80% sequence identity to the amino acid sequence of SEQ ID NO: 45.

9-12. (canceled)

13. The isolated antibody that specifically recognizes FasL of claim 5, wherein the antibody comprises an Fc fragment.

14. The isolated antibody that specifically recognizes FasL of claim 13, wherein the antibody is a full-length IgG antibody.

15. The isolated antibody that specifically recognizes FasL of claim 14, wherein the antibody is a full-length IgG1, IgG2, IgG3, or IgG4 antibody

16. The isolated antibody that specifically recognizes FasL of claim 5, wherein the antibody or antigen binding fragment is a chimeric, humanized or human antibody.

17. The isolated antibody that specifically recognizes FasL of claim 5, is an antigen binding fragment, wherein the antigen binding fragment is selected from the group consisting of a Fab, a Fab′, a F(ab)′2, a Fab′-SH, a single-chain Fv (scFv), an Fv fragment, a dAb, a Fd, a nanobody, a diabody, and a linear antibody.

18. An isolated nucleic acid molecule that encodes the antibody that specifically recognizes FasL of claim 5.

19. A vector comprising the nucleic acid molecule of claim 18.

20. An isolated host cell comprising the isolated antibody that specifically recognizes FasL of claim 5.

21. A method of producing an isolated antibody that specifically recognizes FasL, comprising:
a) culturing the host cell of claim 20 under conditions effective to express the antibody that specifically recognizes FasL; and
b) obtaining the expressed antibody that specifically recognizes FasL from the host cell.

22. A pharmaceutical composition comprising the antibody that specifically recognizes FasL of claim 5, and a pharmaceutically acceptable carrier.

23. The pharmaceutical composition of claim 22, wherein the pharmaceutical composition further comprises other pharmaceutical agents selected from immunosuppressive agents, anti-inflammatory drugs, anti-tumor agents, growth inhibitors, cytotoxic agents, chemotherapy agents or vascular suppressants.

24. (canceled)

25. A method of treating a disease or condition in an individual in need thereof, comprising administering to the individual an effective amount of the antibody of claim 5.

26. The method of claim 25, wherein the disease or condition is an inflammatory disease, cancer or autoimmune disease that associate with FasL-Fas signaling pathway.

27. The method of claim 26, wherein the disease or condition is selected from pemphigus, transplant rejection, graft-versus-host disorders, systemic inflammatory response syndrome, sepsis, multiple organ dysfunction syndrome, acute lung injury, acute respiratory distress syndrome, trauma, multiple sclerosis, idiopathic pulmonary fibrosis, osteoarthritis, inflammatory bowel disease, Crohn's disease, ulcerative colitis, myocardial infarction, cardiomyopathy, ischemic reperfusion injury, diabetes, brain damage, spinal cord injuries, acute viral hepatitis B, acute viral hepatitis C, chronic hepatitis C, chronic hepatitis B, alcoholic hepatitis, nonalcoholic steatohepatitis, cirrhosis, drug-induced liver injury/liver failure, autoimmune hepatitis, chronic kidney disease, acute kidney disease, diabetic nephropathy, cancer.

28. The method of claim 27, wherein the cancer is a FasL-positive cancer.

29. (canceled)

30. The method of claim 25, wherein the method further comprises another agent to an individual in need thereof

31. The method of claim 30, wherein the another agent is selected from immunosuppressive agents, anti-inflammatory drugs, anti-tumor agents, growth inhibitors, cytotoxic agents, chemotherapy agents or vascular suppressants.


SEQUENCE LISTING (58 sequences)

SEQ ID NO: 1 - protein, 7 aa, synthetic construct
GYTFTRF

SEQ ID NO: 2 - protein, 7 aa, synthetic construct
GFTFSSY

SEQ ID NO: 3 - protein, 7 aa, synthetic construct
GYTFTDY

SEQ ID NO: 4 - protein, 4 aa, synthetic construct
PGDG

SEQ ID NO: 5 - protein, 4 aa, synthetic construct
SGGG

SEQ ID NO: 6 - protein, 4 aa, synthetic construct
TYQG

SEQ ID NO: 7 - protein, 8 aa, synthetic construct
LISTAMFD

SEQ ID NO: 8 - protein, 9 aa, synthetic construct
YDNYLYAMD

SEQ ID NO: 9 - protein, 8 aa, synthetic construct
PDWDYAMD

SEQ ID NO: 10 - protein, 11 aa, synthetic construct
SKSVSTSDFSY

SEQ ID NO: 11 - protein, 7 aa, synthetic construct
SQTIGTW

SEQ ID NO: 12 - protein, 11 aa, synthetic construct
SQSVDYDGDSY

SEQ ID NO: 13-15 - skipped (3 entries)

SEQ ID NO: 16 - protein, 6 aa, synthetic construct
SRELPR

SEQ ID NO: 17 - protein, 6 aa, synthetic construct
LYSTPF

SEQ ID NO: 18 - protein, 6 aa, synthetic construct
SRELPY

SEQ ID NO: 19 - protein, 119 aa, synthetic construct
EVQLQQSGVELVKPGASVKLSCKASGYTFTRFDISWVRQRPEQGLEWIGWIFPGDGSSQYNENFKGKATLTTDKSSGTAYMQLNRLTSEDSAVYFCARSLISTAMFDHWGQGTTLTVSS

SEQ ID NO: 20 - protein, 119 aa, synthetic construct
EVQLQQSGPELVKPGASVKLSCKASGYTFTRFDISWVRQRPEQGLEWIGWIFPGDGSTQYNENFKGKATLTTDKSSGTAYMQLNRLTSEDSAVYFCARSLISTAMFDHWGQGTTLTVSS

SEQ ID NO: 21 - protein, 119 aa, synthetic construct
QVQLVQSGAEVKKPGSSVKVSCKASGYTFTRFDISWVRQAPGQGLEWIGWIFPGDGSSQYNENFKGRATLTTDKSTSTAYMELSSLRSEDTAVYFCARSLISTAMFDHWGQGTLVTVSS

SEQ ID NO: 22 - protein, 119 aa, synthetic construct
QVQLVQSGAEVKKPGSSVKVSCKASGYTFTRFDISWVRQAPGQGLEWMGWIFPGDGSSQYNENFKGRATLTTDKSTSTAYMELSSLRSEDTAVYFCARSLISTAMFDHWGQGTLVTVSS

SEQ ID NO: 23 - protein, 119 aa, synthetic construct
QVQLVQSGAEVKKPGSSVKVSCKASGYTFTRFDISWVRQAPGQGLEWMGWIFPGDGSSQYNENFKGRATITTDKSTSTAYMELSSLRSEDTAVYFCARSLISTAMFDHWGQGTLVTVSS

SEQ ID NO: 24 - protein, 119 aa, synthetic construct
QVQLVQSGAEVKKPGSSVKVSCKASGYTFTRFDISWVRQAPGQGLEWMGWIFPGDGSSQYNENFKGRVTLTTDKSTSTAYMELSSLRSEDTAVYFCARSLISTAMFDHWGQGTLVTVSS

SEQ ID NO: 25 - protein, 119 aa, synthetic construct
QVQLVQSGAEVKKPGSSVKVSCKASGYTFTRFDISWVRQAPGQGLEWMGWIFPGDGSSQYNENFKGRVTITADKSTSTAYMELSSLRSEDTAVYFCARSLISTAMFDHWGQGTLVTVSS

SEQ ID NO: 26 - protein, 120 aa, synthetic construct
DVQLQESGGGLVQPGGSRKLSCAASGFTFSSYDMSWVRQTPEKRLEWVAFISSGGGSTYYPDTVKGRFTISRDNAKNTLYLQMSSLKSEDSAMYYCGRHYDNYLYAMDYWGQGTSVTVSS

SEQ ID NO: 27 - protein, 120 aa, synthetic construct
EVQLVESGGGLVQPGGSLRLSCAASGFTFSSYDMSWVRQAPGKRLEWVAFISSGGGSTYYPDTVKGRFTISRDNAKNSLYLQMNSLRAEDTAVYYCGRHYDNYLYAMDYWGQGTLVTVSS

SEQ ID NO: 28 - protein, 120 aa, synthetic construct
EVQLVESGGGLVQPGGSLRLSCAASGFTFSSYDMSWVRQAPGKGLEWVAFISSGGGSTYYPDTVKGRFTISRDNAKNSLYLQMNSLRAEDTAVYYCGRHYDNYLYAMDYWGQGTLVTVSS

SEQ ID NO: 29 - protein, 119 aa, synthetic construct
EVQLQQSGAELVRPGVSVKISCKGSGYTFTDYTMHWVKQSHAKSLEWIGVISTYQGDARYNQKFKGKATMTVDKSSSTAYMELARLTSEDSAIYYCARFPDWDYAMDYWGQGTSVTVSS

SEQ ID NO: 30 - protein, 119 aa, synthetic construct
QIQLLQSGAELVRPGVSVKISCKGSGYTFTDYAMHWVKQSHAKSLEWIGVISTYQGDANYNQKFKGKATMTVDKSSSTAYMELARLTSEDSAIYYCARFPDWDYAMDYWGQGTSVTVSS

SEQ ID NO: 31 - protein, 119 aa, synthetic construct
EVQLQQSGAELVRPGVSVKISCKGSGYTFTDYTIHWVKQSHAKSLEWIGVISTYQGDASYNQKFKGKATMTVDKSSSTAYMELARLTSEDSAIYYCARFPDWDYAMDYWGQGTSVTVSS

SEQ ID NO: 32 - protein, 119 aa, synthetic construct
QVQLVQSGAEVKKPGASVKVSCKGSGYTFTDYTIHWVRQAPGQGLEWIGVISTYQGDASYNQKFKGRATMTVDKSTSTAYMELSSLRSEDTAVYYCARFPDWDYAMDYWGQGTLVTVSS

SEQ ID NO: 33 - protein, 119 aa, synthetic construct
QVQLVQSGAEVKKPGASVKVSCKASGYTFTDYTIHWVRQAPGQGLEWIGVISTYQGDASYNQKFKGRATMTVDKSTSTAYMELSSLRSEDTAVYYCARFPDWDYAMDYWGQGTLVTVSS

SEQ ID NO: 34 - protein, 119 aa, synthetic construct
QVQLVQSGAEVKKPGASVKVSCKGSGYTFTDYTIHWVRQAPGQGLEWMGVISTYQGDASYNQKFKGRATMTVDKSTSTAYMELSSLRSEDTAVYYCARFPDWDYAMDYWGQGTLVTVSS

SEQ ID NO: 35 - protein, 119 aa, synthetic construct
QVQLVQSGAEVKKPGASVKVSCKGSGYTFTDYTIHWVRQAPGQGLEWMGVISTYQGDASYNQKFKGRVTMTVDKSTSTAYMELSSLRSEDTAVYYCARFPDWDYAMDYWGQGTLVTVSS

SEQ ID NO: 36 - protein, 119 aa, synthetic construct
QVQLVQSGAEVKKPGASVKVSCKGSGYTFTDYTIHWVRQAPGQGLEWMGVISTYQGDASYNQKFKGRVTMTVDKSTSTVYMELSSLRSEDTAVYYCARFPDWDYAMDYWGQGTLVTVSS

SEQ ID NO: 37 - protein, 119 aa, synthetic construct
QVQLVQSGAEVKKPGASVKVSCKASGYTFTDYTIHWVRQAPGQGLEWMGVISTYQGDASYNQKFKGRVTMTVDTSTSTVYMELSSLRSEDTAVYYCARFPDWDYAMDYWGQGTLVTVSS

SEQ ID NO: 38 - protein, 111 aa, synthetic construct
DIVMTQSPASLAVSLGQRATISCRASKSVSTSDFSYIHWYQQKPGQPPKLLIFLASHLESGVPARFSGSGSGTDFTLNIHPVEEEDAATYYCQHSRELPRTFGGGTKLEIK

SEQ ID NO: 39 - protein, 111 aa, synthetic construct
DIVLTQSPASLAVSLGQRATISCRASKSVSTSDFSYIHWYQQKPGLPPKLLIFLASHLESGVPARFSGSGSGTDFTLNIHPVEEEDAATYYCQHSRELPRTFGGGTKLEIK

SEQ ID NO: 40 - protein, 111 aa, synthetic construct
EIVMTQSPATLSVSPGERATLSCRASKSVSTSDFSYIHWYQQKPGQPPRLLIFLASHLESGIPARFSGSGSGTEFTLTISSLQSEDFAVYYCQHSRELPRTFGQGTKVEIK

SEQ ID NO: 41 - protein, 111 aa, synthetic construct
EIVMTQSPATLSVSPGERATLSCRASKSVSTSDFSYIHWYQQKPGQPPRLLIYLASHLESGIPARFSGSGSGTEFTLTISSLQSEDFAVYYCQHSRELPRTFGQGTKVEIK

SEQ ID NO: 42 - protein, 111 aa, synthetic construct
EIVMTQSPATLSVSPGERATLSCRASKSVSTSDFSYIHWYQQKPGQAPRLLIYLASHLESGIPARFSGSGSGTEFTLTISSLQSEDFAVYYCQHSRELPRTFGQGTKVEIK

SEQ ID NO: 43 - protein, 107 aa, synthetic construct
DIVMTQSPASQSASLGESVTITCLASQTIGTWLAWYQQKPGKSPQLLIYAATSLADGVPSRFSGSGSGTKFSFKISSLQAEDFVSYYCQQLYSTPFTFGGGTKLEIK

SEQ ID NO: 44 - protein, 107 aa, synthetic construct
DIQMTQSPSSLSASVGDRVTITCLASQTIGTWLAWYQQKPGKSPKLLIYAATSLADGVPSRFSGSGSGTDFTLTISSLQPEDFATYYCQQLYSTPFTFGQGTKVEIK

SEQ ID NO: 45 - protein, 107 aa, synthetic construct
DIQMTQSPSSLSASVGDRVTITCLASQTIGTWLAWYQQKPGKAPKLLIYAATSLADGVPSRFSGSGSGTDFTLTISSLQPEDFATYYCQQLYSTPFTFGQGTKVEIK

SEQ ID NO: 46 - protein, 111 aa, synthetic construct
DIVLTQSPASLAVSLGQRATISCKASQSVDYDGDSYMNWYQQKPGQPPKLLIYAASNLESGIPARFSGSGSGTDFTLNIHPVEEEDAATYYCQHSRELPYTFGGGTKLEIK

SEQ ID NO: 47 - protein, 111 aa, synthetic construct
EIVLTQSPASLAVSLGQRATISCKASQSVDYDGDSYMNWYQQKPGQPPKLLIYAASNLESGIPARFSGSGSGTDFTLNIHPVEEEDAATYYCQHSRELPYTFGGGTKLEIK

SEQ ID NO: 48 - protein, 111 aa, synthetic construct
EIVLTQSPASLAVSLGQRATISCKASQSVDYDGDSYMNWYQQKPGQPPKLLIYAASNLESGVPARFSGSGSGTDFTLNIHPVEEEDAATYYCQHSRELPYTFGGGTKLEIK

SEQ ID NO: 49 - protein, 111 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQQSRELPYTFGGGTKLEIK

SEQ ID NO: 50 - protein, 111 aa, synthetic construct
EIVMTQSPATLSVSPGERATLSCKASQSVDYDGDSYMNWYQQKPGQPPRLLIYAASNLESGIPARFSGSGSGTEFTLTISSLQSEDFAVYYCQHSRELPYTFGQGTKLEIK

SEQ ID NO: 51 - protein, 111 aa, synthetic construct
EIVMTQSPATLSVSPGERATLSCKASQSVDYDGDSYMNWYQQKPGQAPRLLIYAASNLESGIPARFSGSGSGTEFTLTISSLQSEDFAVYYCQHSRELPYTFGQGTKLEIK

SEQ ID NO: 52 - protein, 111 aa, synthetic construct
EIVMTQSPATLSVSPGERATLSCKASQSVDYDGDSYMNWYQQKPGQPPRLLIYAASNLESGIPARFSGSGSGTEFTLTISSLQSEDFAVYYCQQSRELPYTFGQGTKLEIK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 107 aa, synthetic construct [sequence omitted]

SEQ ID NO: 56 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 57 - protein, 281 aa, synthetic construct
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 58 - protein, 148 aa, synthetic construct
PSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL